SEQUENCE LISTING (80 sequences)

SEQ ID NO: 1 - protein, 9 aa, Artificial Sequence
  Description of Artificial Sequence Synthetic peptide
GFSFSSSYW

SEQ ID NO: 2 - protein, 9 aa, Artificial Sequence
  Description of Artificial Sequence Synthetic peptide
IYAGSTGTT

SEQ ID NO: 3 - protein, 13 aa, Artificial Sequence
  Description of Artificial Sequence Synthetic peptide
ARGDYNSGWGVNL

SEQ ID NO: 4 - protein, 6 aa, Artificial Sequence
  Description of Artificial Sequence Synthetic peptide
ESISSN

SEQ ID NO: 5 - protein, 10 aa, Artificial Sequence
  Description of Artificial Sequence Synthetic peptide
QCTDYVNSGA

SEQ ID NO: 6 - protein, 5 aa, Artificial Sequence
  Description of Artificial Sequence Synthetic peptide
DYEMH

SEQ ID NO: 7 - protein, 5 aa, Artificial Sequence
  Description of Artificial Sequence Synthetic peptide
DYEIH

SEQ ID NO: 8 - protein, 17 aa, Artificial Sequence
  Description of Artificial Sequence Synthetic peptide
SIHPGSGGTAYAQKFQG

SEQ ID NO: 9 - protein, 5 aa, Artificial Sequence
  Description of Artificial Sequence Synthetic peptide
YDLDY

SEQ ID NO: 10 - protein, 16 aa, Artificial Sequence
  Description of Artificial Sequence Synthetic peptide
RSSESLLHSNGNTYLY

SEQ ID NO: 11 - protein, 16 aa, Artificial Sequence
  Description of Artificial Sequence Synthetic peptide
RSSESLLHSIGKTYLY

SEQ ID NO: 12 - protein, 7 aa, Artificial Sequence
  Description of Artificial Sequence Synthetic peptide
RKSNLAS

SEQ ID NO: 13 - protein, 9 aa, Artificial Sequence
  Description of Artificial Sequence Synthetic peptide
MQQLEYPLT

SEQ ID NO: 14 - protein, 26 aa, Artificial Sequence
  Description of Artificial Sequence Synthetic peptide
PMIVSVKEGGRTRPQVVSLPNMRVQT

SEQ ID NO: 15 - protein, 15 aa, Artificial Sequence
  Description of Artificial Sequence Synthetic peptide
GGGGSGGGGSGGGGS

SEQ ID NO: 16 - DNA, 40 bp, Artificial Sequence
  Description of Artificial Sequence Synthetic oligonucleotide
atgtgcggccgcaccatgcacgtgcgctcgctgcgcgctg

SEQ ID NO: 17 - DNA, 35 bp, Artificial Sequence
  Description of Artificial Sequence Synthetic oligonucleotide
gctctagactagtggcagccacaggcccggaccac

SEQ ID NO: 18 - DNA, 40 bp, Artificial Sequence
  Description of Artificial Sequence Synthetic oligonucleotide
cagaattcgcgggccgcaccatgccattcctgtggctctg

SEQ ID NO: 19 - DNA, 28 bp, Artificial Sequence
  Description of Artificial Sequence Synthetic oligonucleotide
cttctagactatggctgcagcctcctgg

SEQ ID NO: 20 - DNA, 55 bp, Artificial Sequence
  Description of Artificial Sequence Synthetic oligonucleotide
tggacaaggctgatgtggaattcaagagattccacatcagccttgtccttttttc

SEQ ID NO: 21 - DNA, 59 bp, Artificial Sequence
  Description of Artificial Sequence Synthetic oligonucleotide
tcgagaaaaaaggacaaggctgatgtggaatctcttgaattccacatcagccttgtcca

SEQ ID NO: 22 - DNA, 55 bp, Artificial Sequence
  Description of Artificial Sequence Synthetic oligonucleotide
tgcaggttcctggtgtcagattcaagagatctgacaccaggaacctgcttttttc

SEQ ID NO: 23 - DNA, 59 bp, Artificial Sequence
  Description of Artificial Sequence Synthetic oligonucleotide
tcgagaaaaaagcaggttcctggtgtcagatctcttgaatctgacaccaggaacctgca

SEQ ID NO: 24 - DNA, 59 bp, Artificial Sequence
  Description of Artificial Sequence Synthetic oligonucleotide
tcgaactgctgatggacaaatgttcaagagacatttgtccatcagcagttcattttttc

SEQ ID NO: 25 - DNA, 63 bp, Artificial Sequence
  Description of Artificial Sequence Synthetic oligonucleotide
tcgagaaaaaatgaactgctgatggacaaatgtctcttgaacatttgtccatcagcagttcaa

SEQ ID NO: 26 - protein, 219 aa, Artificial Sequence
  Description of Artificial Sequence Synthetic polypeptide
DILMTQAAPSISVTPGESVSISCRSSESLLHSNGNTYLYWFLQRPGQSPQLLIYRKSNLASGVPDRFSGSGSGTAFTLRISRVEAEDVGVYYCMQQLEYPLTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 27 - protein, 438 aa, Artificial Sequence
  Description of Artificial Sequence Synthetic polypeptide
QVQLQQSGAELVRPGASVKLSCKALGYTFADYEMHWVRQTPVHGLEWTGSIHPGSGGTAYDQRFKGKATLTADKSSSTAYMELSSLTSEDSAVYYCTFYDLDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 28 - protein, 219 aa, Artificial Sequence
  Description of Artificial Sequence Synthetic polypeptide
DILMTQAAPSISVTPGESVSISCRSSESLLHSNGNTYLYWFLQRPGQSPQLLIYRKSNLASGVPDRFSGSGSGTAFTLRISRVEAEDVGVYYCMQQLEYPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 444 aa, Artificial Sequence
  Description of Artificial Sequence Synthetic polypeptide
QVQLQQSGAELVRPGASVKLSCKALGYTFADYEMHWVRQTPVHGLEWTGSIHPGSGGTAYDQRFKGKATLTADKSSSTAYMELSSLTSEDSAVYYCTFYDLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 26 aa, Homo sapiens
PMIVSIKEGGRTRPQVVSLPNMRVQK

SEQ ID NO: 31 - protein, 227 aa, Artificial Sequence
  Description of Artificial Sequence Synthetic polypeptide
QVQLQQSGAELVRPGASVKLSCKALGYTFADYEMHWVRQTPVHGLEWTGSIHPGSGGTAYDQRFKGKATLTADKSSSTAYMELSSLTSEDSAVYYCTFYDLDYWGQGTTLTVSDILMTQAAPSISVTPGESVSISCRSSESLLHSNGNTYLYWFLQRPGQSPQLLIYRKSNLASGVPDRFSGSGSGTAFTLRISRVEAEDVGVYYCMQQLEYPLTFGGGTKLELKRA

SEQ ID NO: 32 - protein, 366 aa, Homo sapiens
MQPLWLCWALWVLPLASPGAALTGEQLLGSLLRQLQLKEVPTLDRADMEELVIPTHVRAQYVALLQRSHGDRSRGKRFSQSFREVAGRFLALEASTHLLVFGMEQRLPPNSELVQAVLRLFQEPVPKAALHRHGRLSPRSARARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLGDYGAQGDCDPEAPMTEGTRCCRQEMYIDLQGMKWAENWVLEPPGFLAYECVGTCRQPPEALAFKWPFLGPRQCIASETDSLPMIVSIKEGGRTRPQVVSLPNMRVQKCSCASDGALVPRRLQP

SEQ ID NO: 33 - protein, 530 aa, Artificial Sequence
  Description of Artificial Sequence Synthetic polypeptide
MGWTLVFLFLLSVTAGVHSRFSQSFREVAGRFLALEASTHLLVFGMEQRLPPNSELVQAVLRLFQEPVPKAALHRHGRLSPRSARARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLGDYGAQGDCDPEAPMTEGTRCCRQEMYIDLQGMKWAENWVLEPPGFLAYECVGTCRQPPEALAFKWPFLGPRQCIASETDSLPMIVSIKEGGRTRPQVVSLPNMRVQKCSCASDGALVPRRLQPGGSPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 34 - protein, 529 aa, Artificial Sequence
  Description of Artificial Sequence Synthetic polypeptide
MGWTLVFLFLLSVTAGVHSRFSQSFREVAGRFLALEASTHLLVFGMEQRLPPNSELVQAVLRLFQEPVPKAALHRHGRLSPRSARARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLGDYGAQGDCDPEAPMTEGTRCCRQEMYIDLQGMKWAENWVLEPPGFLAYECVGTCRQPPEALAFKWPFLGPRQCIASETDSLPMIVSIKEGGRTRPQVVSLPNMRVQKCSCASDGALVPRRLQPSGSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 318 aa, Artificial Sequence
  Description of Artificial Sequence Synthetic polypeptide
MGWTLVFLFLLSVTAGVHSRFSQSFREVAGRFLALEASTHLLVFGMEQRLPPNSELVQAVLRLFQEPVPKAALHRHGRLSPRSARARVTVEWLRVRDDGSNRTSLIDSRLVSVHESGWKAFDVTEAVNFWQQLSRPRQPLLLQVSVQREHLGPLASGAHKLVRFASQGAPAGLGEPQLELHTLDLGDYGAQGDCDPEAPMTEGTRCCRQEMYIDLQGMKWAENWVLEPPGFLAYECVGTCRQPPEALAFKWPFLGPRQCIASETDSLPMIVSIKEGGRTRPQVVSLPNMRVQKCSCASDGALVPRRLQPGGGHHHHHH

SEQ ID NO: 36 - protein, 120 aa, Artificial Sequence
  Description of Artificial Sequence Synthetic polypeptide
QSLEESGGDLVKPEGSLTLTCTASGFSFSSSYWICWVRQAPGKGLERIACIYAGSTGTTYYASWAKGRFTISKTSSTTVTLQMTSLTAADTATYFCARGDYNSGWGVNLWGPGTLVTISS

SEQ ID NO: 37 - protein, 121 aa, Artificial Sequence
  Description of Artificial Sequence Synthetic polypeptide
QEQLEESGGDLVKPEGSLTLTCTASGFSFSSSYWICWVRQAPGKGLERIACIYAGSTGTTYYASWAKGRFTISKTSSTTVTLQMTSLTAADTATYFCARGDYNSGWGVNLWGPGTLVTVSS

SEQ ID NO: 38 - protein, 120 aa, Artificial Sequence
  Description of Artificial Sequence Synthetic polypeptide
QELVESGGDLVKPEGSLTLTCTASGFSFSSSYWICWVRQAPGKGLERIACIYAGSTGTTYYASWAKGRFTISKTSSTTVTLQMTSLTAADTATYFCARGDYNSGWGVNLWGPGTLVTVSS

SEQ ID NO: 39 - protein, 120 aa, Artificial Sequence
  Description of Artificial Sequence Synthetic polypeptide
QSVEESGGDLVKPEGSLTLTCTASGFSFSSSYWICWVRQAPGKGLERIACIYAGSTGTTYYASWAKGRFTISKTSSTTVTLQMTSLTAADTATYFCARGDYNSGWGVNLWGPGTLVTVSS

SEQ ID NO: 40 - protein, 108 aa, Artificial Sequence
  Description of Artificial Sequence Synthetic polypeptide
ALVMTQTPSSASEPVGGTVTIKCQASESISSNLAWYQQKPGQRPKLLIYSASTLASGVPSRFSGSGSGTEFTLTISDLECADAATYYCQCTDYVNSGAFGGGTEVVVK

SEQ ID NO: 41 - protein, 108 aa, Artificial Sequence
  Description of Artificial Sequence Synthetic polypeptide
DVVMTQTPSSASEPVGGTVTIKCQASESISSNLAWYQQKPGQRPKLLIYSASTLASGVPSRFSGSGSGTEFTLTISDLECADAATYYCQCTDYVNSGAFGGGTEVVVK

SEQ ID NO: 42 - protein, 108 aa, Artificial Sequence
  Description of Artificial Sequence Synthetic polypeptide
AIEMTQTPSSASEPVGGTVTIKCQASESISSNLAWYQQKPGQRPKLLIYSASTLASGVPSRFSGSGSGTEFTLTISDLECADAATYYCQCTDYVNSGAFGGGTEVVVK

SEQ ID NO: 43 - protein, 108 aa, Artificial Sequence
  Description of Artificial Sequence Synthetic polypeptide
DVVMTQTASSASEPVGGTVTIKCQASESISSNLAWYQQKPGQRPKLLIYSASTLASGVPSRFSGSGSGTEFTLTISDLECADAATYYCQCTDYVNSGAFGGGTEVVVK

SEQ ID NO: 44 - protein, 108 aa, Artificial Sequence
  Description of Artificial Sequence Synthetic polypeptide
DAVMTQTPSSASEPVGGTVTIKCQASESISSNLAWYQQKPGQRPKLLIYSASTLASGVPSRFSGSGSGTEFTLTISDLECADAATYYCQCTDYVNSGAFGGGTEVVVK

SEQ ID NO: 45 - protein, 466 aa, Artificial Sequence
  Description of Artificial Sequence Synthetic polypeptide
MEWSWVFLFFLSVTTGVHSQSLEESGGDLVKPEGSLTLTCTASGFSFSSSYWICWVRQAPGKGLERIACIYAGSTGTTYYASWAKGRFTISKTSSTTVTLQMTSLTAADTATYFCARGDYNSGWGVNLWGPGTLVTISSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVSSVFIFPPKPKDVLTITLTPKVICVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 46 - protein, 235 aa, Artificial Sequence
  Description of Artificial Sequence Synthetic polypeptide
MSVPTQVLGLLLLWLTDARCDVVMTQTASSASEPVGGTVTIKCQASESISSNLAWYQQKPGQRPKLLIYSASTLASGVPSRFSGSGSGTEFTLTISDLECADAATYYCQCTDYVNSGAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 47 - protein, 49 aa, Artificial Sequence
  Description of Artificial Sequence Synthetic polypeptide
QVQLVQSGAEVDYEMHWVRQAPGQGLEWTGKKPGASVKVSCKASGYTFA

SEQ ID NO: 48 - protein, 30 aa, Artificial Sequence
  Description of Artificial Sequence Synthetic polypeptide
QVQLVQSGGGLVKPGGSVKVSCKASGYTFA

SEQ ID NO: 49 - protein, 30 aa, Artificial Sequence
  Description of Artificial Sequence Synthetic polypeptide
QVQLVQSGAGLVKPGASLKLSCKALGYTFA

SEQ ID NO: 50 - protein, 30 aa, Artificial Sequence
  Description of Artificial Sequence Synthetic polypeptide
QVQLVQSGAGLVKPGASLKLSCKASGYTFA

SEQ ID NO: 51 - protein, 30 aa, Artificial Sequence
  Description of Artificial Sequence Synthetic polypeptide
QVQLVQSGAGVKKPGASLKLSCKASGYTFA

SEQ ID NO: 52 - protein, 30 aa, Artificial Sequence
  Description of Artificial Sequence Synthetic polypeptide
QVQLVQSGAEVKRPGASVKLSCKALGYTFA

SEQ ID NO: 53 - protein, 23 aa, Artificial Sequence
  Description of Artificial Sequence Synthetic peptide
DIVLTQSPSSLSVSPGERVTISC

SEQ ID NO: 54 - protein, 23 aa, Artificial Sequence
  Description of Artificial Sequence Synthetic peptide
DIVMTQTPLSLSVSPGERVTISC

SEQ ID NO: 55 - protein, 23 aa, Artificial Sequence
  Description of Artificial Sequence Synthetic peptide
DIVLTQSPSSVSVTPGQRVTISC

SEQ ID NO: 56 - protein, 23 aa, Artificial Sequence
  Description of Artificial Sequence Synthetic peptide
DIVMTQTPLSLPVTPGQPASISC

SEQ ID NO: 57 - protein, 14 aa, Artificial Sequence
  Description of Artificial Sequence Synthetic peptide
WVRQAPGQGLEWTG

SEQ ID NO: 58 - protein, 14 aa, Artificial Sequence
  Description of Artificial Sequence Synthetic peptide
WVRQAPGKGLEWVG

SEQ ID NO: 59 - protein, 14 aa, Artificial Sequence
  Description of Artificial Sequence Synthetic peptide
WVRQAPVHGLEWTG

SEQ ID NO: 60 - protein, 15 aa, Artificial Sequence
  Description of Artificial Sequence Synthetic peptide
WYQQKPGQAPKLLIY

SEQ ID NO: 61 - protein, 15 aa, Artificial Sequence
  Description of Artificial Sequence Synthetic peptide
WYLQKPGQSPKLLIY

SEQ ID NO: 62 - protein, 15 aa, Artificial Sequence
  Description of Artificial Sequence Synthetic peptide
WFQQRPGQAPKLLIY

SEQ ID NO: 63 - protein, 15 aa, Artificial Sequence
  Description of Artificial Sequence Synthetic peptide
WFLQKPGQPPQLLIY

SEQ ID NO: 64 - protein, 17 aa, Artificial Sequence
  Description of Artificial Sequence Synthetic peptide
SIHPGSGGTAYDQRFKG

SEQ ID NO: 65 - protein, 17 aa, Artificial Sequence
  Description of Artificial Sequence Synthetic peptide
SIHPGSGGTAYDQRFQG

SEQ ID NO: 66 - protein, 32 aa, Artificial Sequence
  Description of Artificial Sequence Synthetic polypeptide
RVTSTRDTSISTAYMELSSLRSDDTVVYYCTF

SEQ ID NO: 67 - protein, 32 aa, Artificial Sequence
  Description of Artificial Sequence Synthetic polypeptide
RVTSTRDTSKNTAYLQLSSLRADDTVVYYCTF

SEQ ID NO: 68 - protein, 32 aa, Artificial Sequence
  Description of Artificial Sequence Synthetic polypeptide
RVTITRDKSKSTAYLELSSLTSEDTAVYYCTF

SEQ ID NO: 69 - protein, 32 aa, Artificial Sequence
  Description of Artificial Sequence Synthetic polypeptide
RVTITADNSKSTAYMELSSLTSEDTAVYYCTF

SEQ ID NO: 70 - protein, 32 aa, Artificial Sequence
  Description of Artificial Sequence Synthetic polypeptide
RVTLTADKSSSTAYMELSSLTSEDTAVYYCTF

SEQ ID NO: 71 - protein, 32 aa, Artificial Sequence
  Description of Artificial Sequence Synthetic polypeptide
GVPDRFSGSGSGTDFTLTISGLQAEDEAVYYC

SEQ ID NO: 72 - protein, 32 aa, Artificial Sequence
  Description of Artificial Sequence Synthetic polypeptide
GVPDRFSGSGSGTDFTLTISRLEAEDFAVYYC

SEQ ID NO: 73 - protein, 32 aa, Artificial Sequence
  Description of Artificial Sequence Synthetic polypeptide
GVPDRFSGSGSGTDFTLKISRVEAEDVAVYYC

SEQ ID NO: 74 - protein, 32 aa, Artificial Sequence
  Description of Artificial Sequence Synthetic polypeptide
GVPDRFSGSGSGTDFTLKISRVEAEDVGVYYC

SEQ ID NO: 75 - protein, 8 aa, Artificial Sequence
  Description of Artificial Sequence Synthetic peptide
QQLEYPLT

SEQ ID NO: 76 - protein, 11 aa, Artificial Sequence
  Description of Artificial Sequence Synthetic peptide
WGQGTTVTVSS

SEQ ID NO: 77 - protein, 11 aa, Artificial Sequence
  Description of Artificial Sequence Synthetic peptide
WGQGTLVTVSS

SEQ ID NO: 78 - protein, 11 aa, Artificial Sequence
  Description of Artificial Sequence Synthetic peptide
WGQGTTLTVSS

SEQ ID NO: 79 - protein, 13 aa, Artificial Sequence
  Description of Artificial Sequence Synthetic peptide
FGGGTKLEIKRTV

SEQ ID NO: 80 - protein, 120 aa, Artificial Sequence
  Description of Artificial Sequence Synthetic polypeptide
QSLEESGGDLVKPEGSLTLTCTASGFSFSSSYWICWVRQAPGKGLERIACIYAGSTGTTYYASWAKGRFTISKTSSTTVTLQMTSLTAADTATYFCARGDYNSGWGVNLWGPGTLVTVSS

(19) United States
(12) Patent Application Publication
(10) Pub. No.: US 2024/0368259 A1
(43) Pub. Date: Nov. 7, 2024

(54) ANTIBODIES AGAINST LEFTY PROTEINS

(71) Applicant: Onena Medicines, S.L., San Sebastian (ES)

(72) Inventors: Neethan Amit Lobo, San Sebastian (ES); Maider Zabala Ugalde, San Sebastian (ES); Errarte Peio Yarza, San Sebastian (ES); Enrique Miranda Rota, San Sebastian (ES); Valentino Bianco, San Sebastian (ES)

(73) Assignee: Onena Medicines, S.L., San Sebastian (ES)

(21) Appl. No.: 18/005,833

(22) PCT Filed: Jul. 16, 2021
(86) PCT No.: PCT/IB2021/000500

§ 371 Date: Jan. 17, 2023

Related U.S. Application Data
(60) Provisional application No. 63/053,473, filed on Jul. 17, 2020.

(51) Int. Cl. C07K 16/22; A61P 35/00

(52) U.S. Cl. CPC C07K 16/22; A61P 35/00; A61K 39/00; C07K 2317/622

(57) ABSTRACT

The present invention relates to antibodies that neutralize the ability of the LEFTY proteins to induce the growth of cancer cells and diagnostic and therapeutic methods involving the same.

### RELATED APPLICATIONS AND INCORPORATION BY REFERENCE

This application claims benefit of and priority to U.S. provisional patent application Ser. No. 63/053,473 filed Jul. 17, 2020. Reference is made to international patent application Serial No. PCT/US2019/027523 filed 15 Apr. 2019, which published as PCT Publication No. WO 2019/200397 on 17 Oct. 2019.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FEDERAL FUNDING LEGEND

This invention was made with government support under Grant Nos. U01CA154209-01 and P01 CA139490-05 awarded by the NIH/NCI and Grant Nos. W81XWH-13-1-0281 and W81XWH-11-1-0287 awarded by the Department of Defense. The government has certain rights in the invention.

### SEQUENCE LISTING

The instant application contains a Sequence Listing which has been submitted electronically in ASCII format and is hereby incorporated by reference in its entirety. Said ASCII copy, created on Aug. 21, 2023, is named U3532-99001_SL.txt and is 64,960 bytes in size.

### FIELD OF THE INVENTION

The present invention relates to antibodies that neutralize the ability of the LEFTY proteins to induce the growth of cancer cells and diagnostic and therapeutic methods involving the same.

### BACKGROUND OF THE INVENTION

Targeting members of the TGFβ superfamily has generated significant interest for therapeutic approaches for fibrosis, inflammation, autoimmunity and cancer (Gordon and Blobe, 2008). These strategies include small molecules that inhibit the kinase activities of TGFβ family member receptors, monoclonal antibodies that neutralize ligand activity and vaccines that inhibit ligand production (de Gramont et al., 2017). Nevertheless, due to the complex nature of the TGFβ superfamily and several other factors, the success of these therapies has been limited. However the convergence onto SMAD signaling (Heldin et al., 1997) underscores the potential of several TGFβ superfamily members to regulate SMAD phosphorylation and function. Recent literature has highlighted the ability of dual-SMAD inhibition to enhance long-term proliferation and self-renewal in somatic cells (Chambers et al., 2009; Mou et al., 2016) and the neural conversion of human embryonic stem cells (Chambers et al., 2009). Since these observations were mediated by synthetic inhibition of SMADs, Applicants hypothesized that physiological dual-SMAD inhibition plays a critical role in regulating the long-term proliferation of epithelial stem cells. Hence, Applicants sought to understand whether select TGFβ family proteins modulate long-term proliferation and self-renewal by acting as natural inhibitors of dual-SMAD signaling. Most TGFβ pathway members can be divided into the TGFβ and BMP subfamilies that are often shown to have opposing effects (David and Massague, 2018). BMP7 is a pleiotropic BMP subfamily member that regulates fundamental developmental processes (Boon et al., 2011; Yco and Whitman, 2001). In the mammary gland, BMP7 has been linked to epithelial-to-mesenchymal transition in mammary ductal epithelial cells (Zeisberg et al., 2003). Depending on the cancer cell line, BMP7 acts as an enhancer or suppressor of invasiveness and proliferation (Alarmo and Kallioniemi, 2010; Buijs et al., 2007; Buijs et al., 2012; Ketolainen et al., 2010; Naber et al., 2012; Wang et al., 2007).

LEFTY proteins are naturally-occurring secreted inhibitors of NODAL/SMAD2 signaling since they prevent assembly of NODAL receptor complex formation by binding and sequestering either NODAL itself or the EGF-CFC co-receptors CRIPTO or CRYPTIC which are necessary for Nodal signaling activation (Chen and Shen, 2004). NODAL and CRIPTO-1 are regulatory factors for mammary gland differentiation and maturation (Strizzi et al., 2008). Since imbalanced SMAD signaling (Kirsammer et al., 2014) is implicated in breast cancer (Moses and Barcellos-Hoff, 2011), Applicants sought to explore how two NODAL antagonists, LEFTY1 and BMP7, affect SMAD signaling in normal and malignant mammary epithelial cell models.

Citation or identification of any document in this application is not an admission that such document is available as prior art to the present invention.

### SUMMARY OF THE INVENTION

The present invention relates to an antibody or antigen binding fragment thereof that binds to a LEFTY protein, such as but not limited to LEFTY1, LEFTY2, LEFTY-A and LEFTY-B, and inhibits the growth of cancer cells, such as breast cancer cells. The present invention encompasses antibodies that neutralize the ability of the LEFTY proteins, such as but not limited to LEFTY1, LEFTY2, LEFTY-A and LEFTY-B.

Applicants have data from commercially available anti-LEFTY1 antibodies that are able to either block or not block the proliferation of two different breast cancer cell lines to gain insight into which specific part of LEFTY1 is necessary to be blocked to reduce breast cancer cell growth. Therefore, the present invention relates to methods of identifying additional anti-LEFTY1 antibodies with specific regions of LEFTY1 identified by Applicants.

The present invention relates to an antibody or antigen binding fragment thereof, which may comprise a heavy chain variable region and a light chain variable region, wherein the heavy chain variable region may comprise heavy chain CDRs which may comprise the amino acid sequences GFSFSSSYW (SEQ ID NO: 1), IYAGSTGTT (SEQ ID NO: 2) and ARGDYNSGWGVNL (SEQ ID NO: 3), and wherein the light chain variable region may comprise light chain CDRs which may comprise the amino acid sequences of ESISSN (SEQ ID NO: 4), SAS and QCTDYVNSGA (SEQ ID NO: 5). In another embodiment, present invention relates to an antibody or antigen binding fragment thereof, which may comprise a heavy chain variable region and a light chain variable region, wherein the heavy chain variable region may comprise the amino acid sequences DYEMH (SEQ ID NO: 6) or DYEIH (SEQ ID NO: 7), SIHPGSGGTAYAQKFQG (SEQ ID NO: 8) and YDLDY (SEQ ID NO: 9), and wherein the light chain variable region may comprise light chain CDRs comprising the amino acid sequences of RSSESLLHSNGNTYLY (SEQ ID NO: 10) or RSSESLLHSIGKTYLY (SEQ ID NO: 11), RKSNLAS (SEQ ID NO: 12) and MQQLEYPLT (SEQ ID NO: 13).

In one embodiment, the heavy chain variable region may comprise a sequence at least 95% identical to the heavy chain variable region of FIG. 25A and the light chain variable region may comprise a sequence at least 95% identical to the light chain variable region of FIG. 25A. The heavy chain may comprise a sequence at least 95% identical to the heavy chain of FIG. 27 and the light chain may comprise a sequence at least 95% identical to the light chain of FIG. 27.

In one embodiment, the heavy chain variable region may comprise a sequence at least 95% identical to the heavy chain variable region of FIG. 35 and the light chain variable region may comprise a sequence at least 95% identical to the light chain variable region of FIG. 35. The heavy chain may comprise a sequence at least 95% identical to the heavy chain of FIG. 35 and the light chain may comprise a sequence at least 95% identical to the light chain of FIG. 35.

In another embodiment, the heavy chain variable region and the light chain variable region may be separated by a linker. The heavy chain variable region and the light chain variable region may be in a single polypeptide chain. The antibody or antigen binding fragment thereof may be a (i) a single chain Fv fragment (scFv); (ii) a dimeric scFv (di-scFv); (iii) a trimeric scFv (tri-scFv); (iv) any one of (i), (ii) or (iii) linked to a constant region of an antibody, Fc or a heavy chain constant domain C_{H2} and/or C_{H3}.

In another embodiment, the heavy chain variable region and the light chain variable region may be in a separate polypeptide chain. More particularly, the antibody or antigen binding fragment thereof may be a (i) a diabody; (ii) a triabody; (iii) a tetrabody; (iv) a Fab; (v) a F(ab′)₂; (vi) a Fv; (vii) one of (i) to (vi) linked to a constant region of an antibody, Fc or a heavy chain constant domain C_{H2} and/or C_{H3}; or, (viii) an intact antibody.

In another embodiment, the antibody or antigen binding fragment thereof may be conjugated to another compound.

In another embodiment, the antibody or antigen binding fragment thereof may bind to the C-terminal region of the LEFTY1 protein. The C-terminal region may be residues 320 to 366 of the LEFTY1 protein.

In another embodiment, the antibody or antigen binding fragment may be a neutralizing antibody. In particular, the neutralizing antibody may comprise a mutated Fc.

The present invention encompasses a nucleic acid encoding any of the herein disclosed antibodies or antigen binding fragments thereof. The invention encompasses an expression construct which may comprise the nucleic acid encoding any of the herein disclosed antibodies or antigen binding fragments thereof. The invention also includes an isolated or recombinant cell expressing any of the herein disclosed antibodies or antigen binding fragments thereof, a nucleic acid encoding any of the herein disclosed antibodies or antigen binding fragments thereof or an expression vector which may comprise a nucleic acid encoding any of the herein disclosed antibodies or antigen binding fragments thereof. The invention also may comprise a composition which may comprise the antibody or antigen binding fragment thereof, the nucleic acid, the expression vector, or the cell comprising the expression vector and a pharmaceutically acceptable carrier.

The invention encompasses a method of treating cancer in a subject, which may comprise administering to the subject and effective amount of the herein disclosed antibody or antigen binding fragment thereof, a nucleic acid encoding the same, an expression vector with the nucleic acid or a composition which may comprise the antibody or antigen binding fragment thereof, the nucleic acid, the expression vector, or the cell comprising the expression vector and a pharmaceutically acceptable carrier.

The present invention encompasses a use of the herein disclosed antibody or antigen binding fragment thereof, a nucleic acid encoding the same, an expression vector with the nucleic acid or a composition which may comprise the antibody or antigen binding fragment thereof, the nucleic acid, the expression vector, or the cell comprising the expression vector and a pharmaceutically acceptable carrier in the manufacture of a medicament for treating cancer.

The present invention encompasses the herein disclosed antibody or antigen binding fragment thereof, a nucleic acid encoding the same, an expression vector with the nucleic acid or a composition which may comprise the antibody or antigen binding fragment thereof, the nucleic acid, the expression vector, or the cell comprising the expression vector and a pharmaceutically acceptable carrier for use in treating cancer.

The invention also includes a method for increasing expression or activating SMAD2 and/or SMAD5 in a subject, the method which may comprise administering to the subject and effective amount of the herein disclosed antibody or antigen binding fragment thereof, a nucleic acid encoding the same, an expression vector with the nucleic acid or a composition which may comprise the antibody or antigen binding fragment thereof, the nucleic acid, the expression vector, or the cell comprising the expression vector and a pharmaceutically acceptable carrier.

The invention also includes a method for neutralizing a LEFTY protein in a subject, the method which may comprise administering to the subject and effective amount any the herein disclosed antibody or antigen binding fragment thereof, a nucleic acid encoding the same, an expression vector with the nucleic acid or a composition which may comprise the antibody or antigen binding fragment thereof, the nucleic acid, the expression vector, or the cell comprising the expression vector and a pharmaceutically acceptable carrier. The LEFTY protein may be LEFTY1, LEFTY2, LEFTY-A or LEFTY-B.

The cancer may be breast invasive carcinoma, liver carcinoma, cholangiocarcinoma, uterine carcinoma, ovarian carcinoma, melanoma, thymoma, lung adenocarcinoma, pheochromocytoma/paraganglioma, esophageal carcinoma, pancreatic carcinoma, glioblastoma multiforme, colorectal carcinoma, renal cell carcinoma or adrenal carcinoma.

The invention also includes administering a cancer drug in combination with the herein disclosed antibody or antigen binding fragment thereof, a nucleic acid encoding the same, an expression vector with the nucleic acid or a composition which may comprise the antibody or antigen binding fragment thereof, the nucleic acid, the expression vector, or the cell comprising the expression vector and a pharmaceutically acceptable carrier. The cancer drug may be docetaxel or pembrolizumab.

The invention also includes a method of eliciting an immune response in a subject, the method which may comprise administering to the subject an effective amount of an antigen which may comprise a C-terminal region of the LEFTY1 protein. The invention also includes an antigen which may comprise a C-terminal region of the LEFTY1 protein use in eliciting an immune response. The invention also includes use of an effective amount of an antigen which may comprise a C-terminal region of the LEFTY1 protein in the manufacture of a medicament for eliciting an immune response. The C-terminal region may be residues 320 to 366 of the LEFTY1 protein. The antigen may be combined with an adjuvant. The antigen of claim 33, wherein the C-terminal region may be residues 320 to 366 of the LEFTY1 protein.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

The following detailed description, given by way of example, but not intended to limit the invention solely to the specific embodiments described, may best be understood in conjunction with the accompanying drawings.

FIGS. 1A-1D. LEFTY1 expression is restricted to a subset of luminal cells and rare basal cells while BMP7 is secreted predominantly by basal cells. (FIG. 1A) Left panel is Representative FACS plot of the mouse mammary epithelial cell subpopulations stained with the indicated cell surface markers. The cells were lineage-depleted for TER119+, CD45+ and CD31+ cells followed by dead and doublet cell exclusion. Different subpopulations are indicated in colored legends. Right panel shows Principal component analysis reveals different subpopulations based on single-cell PCR analysis. (FIG. 1B) Hierarchically clustered analysis of single-cell PCR analysis of the populations sorted from mouse mammary epithelial cells. (FIG. 1C) Representative images of BMP7 protein staining in frozen sections of mammary glands isolated from 6-8 weeks old w.t. mice. Scale bar 20 μm. (FIG. 1D) Representative images of LEFTY1 protein staining in frozen sections of mammary glands isolated from 6-8 weeks old w.t. mice. Scale bar 20 μm. Sec also FIGS. 8A-8H.

FIGS. 2A-2N. LEFTY1 induces and BMP7 reduces ductal branching and the long-term proliferative potential of mammary progenitor cells. (FIG. 2A) Schematic representation of the procedure used to inject control, LEFTY1- or BMP7-secreting L1 fibroblasts into the mammary fat-pad followed by analysis of mammary glands collected. (FIG. 2B) Quantification of branches counted in whole mount staining of the glands exposed to LEFTY1 (n=6) or control groups (n=7). Statistical analysis, Student's T-test, *** refers to p<0.001. Data are represented as mean±S.E.M. (FIG. 2C) Quantification of branches counted in whole mount staining of the glands exposed to BMP7 (n=6) or control groups (n=7). Statistical analysis, Student's T-test, * refers to p<0.05. Data are represented as mean±S.E.M. (FIG. 2D) Representative images of the whole mount staining of the indicated groups of mice with additional magnification of the demarcated area. Magnification 40×. (FIG. 2E) Cells enriched in highly proliferative mammary epithelial cells were grown in the 3D organotypic assays to test their organoid formation potential. Representative images of the colonies grown in the presence of BMP7, LEFTY1 or control (n=7). Scale bar 100 μm. (FIG. 2F) Cells enriched in highly proliferative mammary epithelial cells were isolated from w.t. adult mice and sorted using flow cytometry and grown in the 3D organotypic assays to test their organoid formation potential in presence of increasing doses of BMP7. Statistical analysis, Student's T-test, ** refers to p<0.01. *** refers to p<0.001. (FIG. 2G) Cells enriched in highly proliferative mammary epithelial cells were grown in the 3D organotypic assays to test their organoid formation potential in the presence of LEFTY1 and/or CRIPTO-1 (n=7). Data are represented as mean±S.D. One-way ANOVA with Dunnet's adjustment, ** refers to P<0.01; refers to P<0.001. (FIG. 2H) Cells enriched in highly proliferative mammary epithelial cells were grown in the 3D organotypic assays to test their organoid formation potential in the presence of BMP7, LEFTY1 and/or CRIPTO-1 (n=7). Data are represented as mean±S.D. One-way ANOVA with Dunnet's adjustment, * refers to P<0.05; ** refers to P<0.01; *** refers to P<0.001. (FIG. 21) Lefty1 knocked-down showed that the basal CD49f^{hi}CD24^{med} transplantable mammary epithelial cells (Left panel) and the CD49f^{low}CD24^{low} myoepithelial cells (Right panel) relied on LEFTY1 production to form mammary organoids (n=3). Data are represented as mean±S.D. One-way ANOVA with Dunnet's adjustment, * refers to P<0.05; ** refers to P<0.01; *** refers to P<0.001. (FIG. 2J) Control, LEFTY1- or BMP7-expressing mammary fibroblasts were injected in the proximity of the mammary gland of 14-18 weeks old NSG female mice. Two to three weeks later, the mammary epithelial cells were isolated and injected in a limiting dilution fashion into weaning age donor mice to assess their engraftment potential. (FIG. 2K) Extreme limiting dilution analysis (ELDA) from the mammary outgrowth formation of the indicated groups showed that the mammary epithelial cells isolated from the glands that were exposed to LEFTY1 had a significant engraftment advantage compared to control or BMP7 treated ones. Data are represented as mean±S.D. * refers to P<0.05. (FIG. 2L) Mammary epithelial cells of adult w.t. female mice were isolated and infected with different vectors encoding different shRNAs against Lefty1 or control virus. The next day, the transduced cells were injected in the cleared fat-pad of weaning age mice in order to test their exogenous mammary tree repopulation ability. Next, the newly formed mammary glands were dissociated and injected into secondary weaning age donors to test their long-term proliferation potential. (FIG. 2M and FIG. 2N) ELDA results showed that knocking down Lefty1 significantly impairs the primary and secondary engraftment of mammary epithelial cells. The effect of the shRNA Lefty1 #2 was so pronounced that Applicants were no primary outgrowths. Data are represented as mean±S.D. * refers to P<0.05. See also FIGS. 9A-11F.

FIGS. 3A-3K. LEFTY1 simultaneously suppresses SMAD2 and SMAD5 in basal cells. (FIG. 3A) Quantification of pSMAD2 positive cells within the luminal and basal compartments of mammary glands isolated from mice that were injected with control or LEFTY1-secreting fibroblasts. Data are represented as mean±S.D. Statistical analysis, Student's T-test, **** refers to p<0.0001. (FIG. 3B) Quantification of pSMAD2 positive cells within the luminal and basal compartments of mammary glands isolated from mice that were injected with control or BMP7-secreting fibroblasts. Data are represented as mean±S.D. Statistical analysis, Student's T-test, n.s. refers to non-significant. (FIG. 3C) Quantification of pSMAD5 positive cells within the luminal and basal compartments of mammary glands isolated from mice that were injected with control or LEFTY1-secreting fibroblasts. Data are represented as mean±S.D. Statistical analysis, Student's T-test, * refers to p<0.05; **** refers to p<0.0001. (FIG. 3D) Quantification of pSMAD5 positive cells within the luminal and basal compartments of mammary glands isolated from mice that were injected with control or BMP7-secreting fibroblasts. Data are represented as mean±S.D. Statistical analysis, Student's T-test, n.s. refers to non-significant; * refers to p<0.05. (FIG. 3E) Representative images of the quantified sections for pSMAD2 analysis LEFTY1-secreting fibroblasts transplanted mammary glands. Scale bar 20 μm. (FIG. 3F) Representative images of the quantified sections for pSMAD5 analysis LEFTY1-secreting fibroblasts transplanted mammary glands. Scale bar 20 μm. (FIG. 3G) Representative images of the quantified sections for pSMAD2 analysis BMP7-secreting fibroblasts transplanted mammary glands. Scale bar 20 μm. (FIG. 3H) Representative images of the quantified sections for pSMAD5 analysis BMP7-secreting fibroblasts transplanted mammary glands. Scale bar 20 μm. (FIG. 3I) Western-blot for COMMA-D mouse mammary epithelial cells for pSMAD2, pSMAD5, total SMAD2/3 or SMAD1/5/8 antibodies. COMMA-D cells were serum starved 16 hours and blocked with LEFTY1 (200 ng/ml) for 1 hour and stimulated with BMP7 (50 ng/ml) and Nodal (50 ng/ml) for 30 minutes in serum free media (n=3). (FIG. 3J) Quantification of the pSMAD5 signal detected by WB in the indicated conditions. (FIG. 3K) Quantification of the pSMAD2 signal detected by WB in the indicated conditions. See also FIGS. 12A-12I.

FIGS. 4A-4E. LEFTY1 directly binds to the BMP receptor BMPR2. (FIG. 4A) Relative mRNA levels of Bmpr2 shows that basal cells (CD49f^{hi}CD24^{med} and CD49f^{low}CD24^{low}) express significantly higher levels than the CD49f^{low}CD24^{hi} and CD49f^{low}/−CD24^{med} luminal cells. Gapdh was used as a housekeeping gene. Statistical analysis is one-way ANOVA with Dunnet's adjustment with ** representing significance between Bmpr2 in (CD49f^{hi}CD24^{med} and CD49f^{low}CD24^{low}) basal cells and (CD49f^{low}CD24^{hi} and CD49f^{low}/−CD24^{med}) luminal cells. (FIG. 4B) Representative images of target mediated ligation assay show the interaction between LEFTY1 and BMPR2. Red puncta indicate positive interactions. Scale bar 10 μm. (FIG. 4C) Quantification of the positive interaction between BMP7 (used as a positive control), and LEFTY1 with BMPR2. Ten different fields were counted per condition (n=3). Statistical analysis is one-way ANOVA with Dunnet's adjustment, **** refers to P<0.0001. (FIG. 4D) Western-blot of the input, co-immunoprecipitated beads (control), IgG (control), LEFTY1, and BMPR2, shows that LEFTY1 is able to pull-down BMPR2 (150 KDa) and reciprocally BMPR2 is able to pull down LEFTY1 (pro-protein 41 kDa, mature form 30 kDa). (FIG. 4E) Schematic representation of the mechanism by which LEFTY1 inhibits SMAD5 and SMAD2 phosphorylation by binding BMPR2 and preventing its interaction with BMP7 and Nodal. See also FIGS. 12A-12I.

FIGS. 5A-5F. Attenuated LEFTY1 abrogates the proliferation of breast cancer cells in vivo. (FIG. 5A) Percentage of genetic alterations in LEFTY1 found in METABRIC (indicated in blue) and TCGA (indicated in red) human breast cancer datasets. The number of patients analyzed was 2867. (FIG. 5B) Characterization of LEFTY1 copy number of LEFTY1 in the five different breast cancer PDX models used in this study. (FIG. 5C) Characterization of LEFTY1 mRNA levels in the five different breast cancer PDX models used in this study. (FIG. 5D) Characterization of LEFTY1 protein levels by immunofluorescence in the five different breast cancer PDX models used in this study. Scale bar 100 μm. (FIG. 5E) Western blot analysis shows the knock-down efficiency of the shLEFTY1 #2 (shL2) and shLEFTY1 #3 (shL3) virus measured by decrease in LEFTY1 protein in MDA-MD-157 breast tumor cell line. (FIG. 5F) Size of the tumors arising from control infected or breast tumor cells that were infected with two virus that knocks down LEFTY1 (shLEFTY1 #2-shL2- and shLEFTY1 #3-shL3-) were normalized to infection % as measured by RFP %. Each circle represents a single tumor normalized to RFP % as elaborated in FIG. 19A. This experiment was repeated twice and in each group 10 to 20 mice were studied. See also FIGS. 13A-13I.

FIGS. 6A-6E. Autocrine LEFTY1 is necessary for the proliferation tumor initiating breast cancer cells. (FIG. 6A) LEFTY1 mRNA expression levels in the tumorigenic (TG) and non-tumorigenic (NTG) populations sorted based on CD49f and EpCAM expression. The populations were sorted from five different PDX models (n=2-3 independent tumors per PDX model). (FIG. 6B) BMP7 mRNA expression levels in the tumorigenic (TG) and non-tumorigenic (NTG) populations sorted based on CD49f and EpCAM expression. The populations were sorted from five different PDX models (n=2-3 independent tumors per PDX model). (FIG. 6C) Quantification of the number of breast tumor organoid formation from in vitro LDA experiments in which the cancer cells were isolated from PDX1, PDX3, PDX4 and PDX5 and infected with the indicated virus. The number of colonies was normalized by size. Statistical analysis is one-way ANOVA with Dunnet's adjustment, **** refers to P<0.0001. (FIG. 6D) ELDA analysis of the frequency of tumor initiating cells upon LEFTY1 genetic knock-down in the PDX1 model. Tumor initiating cell frequency and 95% CI data for each group. **** refers to p<0.0001. (FIG. 6E) ELDA analysis of the frequency of tumor initiating cells upon LEFTY1 genetic knock-down in the PDX5 model. Tumor initiating cell frequency and 95% CI data for each group. * refers to p<0.05; *** refers to p<0.001. Sec also FIGS. 13A-13I.

FIGS. 7A-7G. LEFTY1 binds to BMPR2 and is a concomitant SMAD2 and SMAD5 inhibitor in tumor-initiating cells. (FIG. 7A) Representative pictures of cells from isolated TG and NTG to assess LEFTY1-BMPR2, BMP7-BMRP2 interaction. Scale bar 20 μm. (FIG. 7B) Quantification by proximity ligation of LEFTY1-BMPR2 or BMP7-BMPR2 interaction in tumorigenic cells (TG, enriched by the CD49f₊EpCAM⁺phenotype) or non-TGs (NTG, defined by non-CD49f⁺EpCAM⁺) sorted cells of PDX1, PDX2 and PDX3. Ten fields per condition were analyzed to quantify the interactions. (FIG. 7C) Representative images of cells from isolated TG and NTG stained to assess pSMAD5 staining. IgG staining and treatment with SMAD pathway inhibitor LDN-193189 were used as negative controls. Scale bar 100 μm. (FIG. 7D) Quantification of pSMAD2 and pSMAD5 protein levels in TG and NTG sorted cells from PDX1, PDX2 and PDX3. Ten fields per condition were analyzed to quantify the interactions and pSMAD stainings. Data are represented as mean±S.D. Statistical analysis, Student's T-test. **** refers to p<0.0001. (FIG. 7E) Immunoblotting of cells from isolated TG and NTG and treated with BMP7 and/or LEFTY1 to assess pSMAD5 response. (FIG. 7F) Immunoblotting of breast cancer cell line MDA-MB-231 upon treatment with BMP7 and/or LEFTY1 to assess pSMAD5 response. (FIG. 7G) Schematic representing the TG cells where LEFTY1-BMPR2 interaction is predominant whereas in NTG, BMP7-BMPR2 will be more frequent. pSMAD2 and pSMAD5 levels will be directly affected in these cell types depending on the type of interaction.

FIGS. 8A-8H, related to FIG. 1: Protein and single-cell transcriptional analysis of adult mammary epithelial cells. (FIG. 8A) Sorting strategy that Applicants used in Example 1 to isolate the different populations isolated from normal mouse mammary gland. The mammary epithelial cells are gated on lineage negative (CD45−, CD31−, Ter119−), DAPI negative cells and then sorted based on CD24 and CD49f expression. (FIG. 8B) Lefty1 expression across developmental times (color coded from high expression in yellow to no expression in dark blue) and Lefty1 expression across different cell types (each dot indicates a single cell) and (FIG. 8C) Bmp7 expression according to the same display parameters as. Data is obtained from the Marioni and Khaled labs, (Bach et al., Nat Comm, 2017 https://marionilab.cruk.cam.ac.uk/mammaryGland/). Their data is drawn from cells obtained from following 4 different developmental stages: NP (nulliparous), G (gestational), L (lactation), PI (post involution). Dendrogram of clusters based on the log-transformed mean expression values of Lefty1 or Bmp7 in the 15 clusters defined by Bach et. al: Cluster of luminal cells: c1: Hsp-PI; c2: Hsp-NP; c3: Hsd-PI; c4: Hsd-NP; c5: Hsd-G; c6: Lp-NP; c7: Lp-PI; c8: Avd-G; c10: Avp-G; c9: Avd-L; c11: Avp-L; cluster of basal cells: c12: Bsl-G; c13: Bsl; c14: Myo; c15: Prc. Abbreviations: Hsp: hormone sensing progenitors; Hsd: hormone sensing differentiated; Lp: luminal progenitor; Avd: differentiated alveolar cells; Avp: progenitor alveolar cells; Bsl: basal cells; Myo: myoepithelial cells; Prc: Procr+ cells; PI: 11 days post natural involution; NP: nulliparous; G: day 14.5 gestation; L: day 6 lactation. (FIG. 8D) Bmp7 expression levels in the indicated populations by single cell expression data from the Tabula Muris dataset (Shaum et al. 2018, Nature, accession https://tabula-muris.ds.czbiohub.org/). First panel shows BMP7 expression data (color coded from dark (high expression) to no expression (gray)). Middle panel shows color coding of clusters of cells based on their indicated cell ontology. Right panel shows violin plots reflecting the distribution of individual cell expression of Bmp7 across the indicated cell types. (FIGS. 8E, F) Bmp2 and Bmp4 expression at single cell level in mammary epithelial cells. Data is obtained from the Marioni and Khaled labs (Bach et al., Nat Comm, 2017https://marionilab.cruk.cam.ac.uk/mammaryGland/). The panels show Bmp2 or Bmp4 expression across different clusters (C(X)) of cluster cell types (each dot indicates a single cell) and Bmp2 or Bmp4 expression across developmental times (right, color coded from high expression in yellow to no expression in dark purple). (FIG. 8G) Immunofluorescence staining for NODAL, LEFTY1 and BMP7 revealed positive staining in different sub-compartments of the mammary epithelial cells. Scale bar 20 μm. (FIG. 8H) Bmp7 and Nodal expression levels in the indicated populations sorted and enriched by flow cytometry. Red is CD49f^{hi}CD24^{med} (MRU), yellow is CD49f^{low}CD24^{low} (Myoepithelial), Green is CD49f^{low}CD24^{hi} (MaCFC) and Blue is CD49f^{-/low}CD24^{med} (mature Luminal cells).

FIGS. 9A-9F, related to FIG. 2: Effects of LEFTY1 and BMP7 in the morphology of the mouse mammary gland. (FIG. 9A) Schematic representation of the procedure used to quantify the number of branching. junctions and average length to the terminal end of mammary ducts of the glands from mice treated with LEFTY1 or BMP7 and control treated mice. (FIG. 9B) Quantification of GFP+ L1 cells persistent in the mammary gland in days 1-16 post injection. Representative images from Day 1 and 16 are also presented. (FIG. 9C) Quantification of the average length of the branches and number of junctions counted in whole mount staining of the glands exposed to LEFTY1 (n=6) and control groups (n=7). Four to seven images per mice were analyzed for quantification. Data are represented as mean±S.E.M. Statistical analysis, Student's T-test, ** refers to p<0.01; **** refers to p<0.0001. (FIG. 9D) Quantification of the average length of the branches and number of junctions counted in whole mount staining of the glands exposed to BMP7 and control groups (n=7). Four to seven images per mice were analyzed for quantification. Data are represented as mean±S.E.M. Statistical analysis, Student's T-test, * refers to p<0.05; ** refers to p<0.01. (FIG. 9E) Representative images of Ki67 staining of the mammary gland sections obtained from the NSG mice in which Applicants injected engineered L1 cells. Scale bar 40 μm. Twenty fields per condition were analyzed to quantify the staining. Data are represented on the right as mean±S.D. Statistical analysis, Student's T-test. * refers to p<0.05; *** refers to p<0.001 refers to p<0.0001. (FIG. 9F) Quantification of different mammary epithelial subpopulations analyzed by FACS after the endogenous mammary gland was exposed to Lefty1 secretion via Applicants' L1-cell transplant system. Data are represented on the right as mean±S.D. Statistical analysis, Student's T-test. * refers to p<0.05; ** refers to p<0.01.

FIG. 10A-10F, related to FIG. 2: Autocrine and paracrine effects of NODAL and LEFTY1 in mammary epithelial cell populations. (FIG. 10A) Cells enriched in highly proliferative mammary epithelial cells were isolated from w.t. adult mice and sorted using flow cytometry. The sorted cells were grown in the 3D organotypic assays to test their organoid formation potential in presence of NODAL. (FIG. 10B) Quantification of the area of the organoids that were formed in the presence of LEFTY1, BMP7 or control. Each dot represents an organoid (n=3). (FIG. 10C) Percentage of inhibition of Lefty1 when cells were transfected with different shRNAs against Lefty1. Data are represented as mean±S.D. (FIG. 10D) HEK293T cells were co-transfected with pEGFP-C3 plasmid in which EGFP is fused to the N-terminus of Lefty1, and different shRNAs against Lefty1 cloned into pSICO-R vector. Different images showed the efficiency of the shRNA constructs to decrease the EGFP signal. Scale bar 100 μm. (FIG. 10E) Cells enriched in highly proliferative mammary epithelial cells were isolated from w.t. adult mice and sorted using f^{low} cytometry and infected with shLefty (RFP) or control vector (GFP). The infected cells were grown in the 3D organotypic assays to test their organoid formation potential in combinations of LEFTY-secreting cells (WT Lefty) or Lefty-deficient shLefty cells. Scale bar 100 μm. Bottom panel depicts quantification of shLefty colonies in increasing co-culture does of LEFTY-secreting cells. Data (n=4) are represented as mean±S.D. Statistical analysis, One-way ANOVA with Dunnet's adjustment, refers to p<0.0001. (FIG. 10F) Cells enriched in highly proliferative mammary epithelial cells were isolated from w.t. adult mice and sorted using flow cytometry and infected with shLefty (GFP) or shC control vector (GFP). The infected cells were grown in the 3D organotypic assays to test their organoid formation potential in the presence of Noggin, a BMP7 inhibitor. Scale bar 100 μm. Bottom panel depicts quantification of mammary organoids in the presence or absence of Noggin. Data (n=4) are represented as mean±S.D. Statistical analysis, One-way ANOVA with Dunnet's adjustment, **** refers to p<0.0001.

FIGS. 11A-11F, related to FIG. 2: Lefty1 is required for the long-term proliferation ability of mammary epithelial cells. (FIG. 11A) Adult female mice were injected with control, LEFTY1 or BMP7 expressing mammary fibroblasts into the mammary fat-pad. The endogenous mammary glands were then isolated, and mammary epithelial cells were injected into donor weaning age nice. The number of positive outgrowths from the total of transplanted glands are shown as well as the frequency and 95% CI of engrafting cells calculated by ELDA. Statistical analysis, Chi-square, * refers to P<0.05. (FIG. 11B) In vivo outgrowth formation of mammary epithelial cells that were infected with control virus (HIV-shC) or Lefty1 knocked-down virus (HIV-shLefty1 #1, HIV-shLefty1 #2). Results are pooled from 3 independent experiments. (FIG. 11C) Frequency and 95% CI from the transplant results summarized in B. The frequency and 95% CI were calculated using Extreme limiting dilution analysis (ELDA). Statistical analysis, Chi-square, * refers to P<0.05. (FIGS. 11D and E) Similar analysis done for B and C but the cells injected were isolated from the primary outgrowths described in B and C. The frequency and 95% CI were calculated using Extreme limiting dilution analysis (ELDA). Statistical analysis, Chi-square, * refers to P<0.05. (FIG. 11F) Representative pictures of secondary outgrowth obtained from the indicated groups. Magnification 10×.

FIGS. 12A-12I, related to FIGS. 3 and 4: LEFTY1 inhibits BMP7 signaling through a direct interaction with BMPR2. (FIG. 12A) Western-blot for COMMA-D mouse mammary epithelial cells for pSMAD2, pSMAD5, total SMAD2/3 or SMAD1/5/8 antibodies. COMMA-D cells were serum starved 16 hours and blocked with LEFTY1 (200 ng/ml) for 1 hour and stimulated with TGF-β (20 ng/mL) for 30 minutes in serum free media (n=3). Quantification of the pSMAD5 and pSMAD2 signal detected by WB in the indicated conditions. (FIG. 12B) 3T3-L1 fibroblasts were transfected with pGL3 BRE-luciferase plasmid. 48 hours after transfection, cells were washed and incubated with different concentrations of BMP7 (from 0-800 ng/ml) for 16 hours prior to collection of the cells. Data are represented as mean±S.D. (FIG. 12C) Same as in (B) but the cells were grown in the presence of LEFTY1. Data are represented as mean±S.D. (FIG. 12D) Same as in (B) but the cells were grown in the presence of BMP7 and LEFTY1 at the indicated concentrations. Data are represented as mean±S.D. Statistical analysis, one-way ANOVA with Dunnet's adjustment, * refers to p<0.05. (FIG. 12E) Relative expression of different Type I and Type II TGFβ family receptors from the indicated populations that were isolated from mammary glands of 3 independent adult w.t. female mice. The expression was normalized to Gapdh. Statistical analysis is one-way ANOVA with Dunnet's adjustment, ** represents significance (p<0.01) between Acvr2a in (CD49f^{hi}CD24^{med} and CD49f^{low}CD24^{low}) basal cells and (CD49f^{low}CD24^{hi} and CD49f^{low}/−CD24^{med}) luminal cells; **** represents significance (p<0.0001) between Bmpr1b in (CD49f^{hi}CD24^{med} and CD49f^{low}CD24^{low}) basal cells and (CD49f^{low}CD24^{hi} and CD49f^{low/-}CD24^{med}) luminal cells. (FIG. 12F) The expression of the keratins Krt14 and Kr18 from the sorted populations used as sorting quality control. The expression was normalized to Gapdh. **** refers to p<0.0001 between Krt14 or Krt18 in (CD49f^{hi}CD24^{med} and CD49f^{low}CD24^{low}) basal cells and (CD49f^{low}CD24^{hi} and CD49f^{low/-}CD24^{med}) luminal cells. (FIG. 12G) Quantitation of target mediated ligation assay to evaluate the interaction between LEFTY1 and different Type I and Type II TGFβ family receptors. Ten different fields were counted per condition (n=3). **** refers to p<0.0001. (FIGS. 12H, I) Quantification and representative images of Lefty-BMPR2, BMP7-BMPR2 interactions and pSMAD5 staining of breast cancer cell line MDA-MB-231 upon treatment with LEFTY1, BMP7 and increasing doses of Lefty Blocking Peptide (Lefty BP). Scale bar 100 μm. Twenty different fields were counted per condition (n=3).

FIGS. 13A-13I, related to FIGS. 5, 6: Characterization of TG and NTG compartments of breast PDX models for Type I and Type II TGFβ family receptors and anti-tumor effect of LEFTY1 in primary breast cancer. (FIG. 13A) Tumors from FIG. 5F are represented with tumor volume normalized to RFP % to calculate the effect of knockdown of LEFTY1 on tumor growth. (FIG. 13B) FACS analysis of the strategy used to sort the different populations isolated from different breast cancer PDX models characterized in the lab. The breast cancer tumor cells are gated on lineage negative (CD45−, CD31−, CD4−, CD16−, CD64−), DAPI negative cells and the sorted based on EpCAM and CD49f expression. All populations were double sorted, and purity checked. (FIG. 13C) Analysis of the mRNA expression levels of the indicated genes in the tumorigenic (TG) and non-tumorigenic (NTG) populations sorted based on CD49f and EpCAM expression. The populations were sorted from the five different PDX models indicated in Table 1 and 3 independent tumors were used per PDX model. All these models have been previously characterized to follow the cancer stem cell paradigm. Data are represented as mean±S.D. Statistical analysis, Student's T-test; unless stated analysis is non-significant with p>0.05. (FIG. 13D) ELDA analysis of the frequency of tumor initiating cells upon LEFTY1 genetic knock-down in the PDX1 model. (FIG. 13E) Tumor weights size at the end point of the PDX1 in vivo study. Data are represented as mean±S.D. Statistical analysis, one-way ANOVA with Dunnet's adjustment. * refers to p<0.05; **** refers to p<0.0001. (FIG. 13F) In vivo tumor growth kinetics progression of the specific number of PDX1 cancer cells infected with the indicated virus. (FIG. 13G) ELDA analysis of the frequency of tumor initiating cells upon LEFTY1 genetic knock-down in the indicated PDX5 model. (FIG. 13H) Tumor weights size at the end point of the PDX5 in vivo study. Data are represented as mean±S.D. Statistical analysis, one-way ANOVA with Dunnet's adjustment. ** refers to p<0.01. (FIG. 13I) In vivo tumor growth kinetics progression of the specific number of PDX5 cancer cells infected with the indicated virus.

FIG. 14 show that the MCF7, MDA-231, and CA51 breast cancer cell lines express LEFTY1 and LEFTY2 proteins. These data show that LEFTY1 and LEFTY2 are expressed by hormone receptor positive and triple negative breast cancer cells.

FIG. 15 shows that Lefty1 and Lefty2 and Cripto (Cripto1, TDGF1) synergize to increase the growth of MCF7 breast cancer cells. These data show that LEFTY1/2 and CRIPTO decrease the proliferation of hormone responsive breast cancer cells. However, the combination of both LEFTY1/2 and CRIPTO together increase the proliferation of hormone responsive breast cancer cells. This effect is dependent on the ratio of the two proteins, observed by the effect at 50 ng/ml and not at higher concentrations of the two proteins.

FIG. 16 shows that Lefty1 and Lefty 2 and Cripto (Cripto1, TDGF1) synergize to increase the growth of MDA-MB-231 breast cancer cells. These data show that LEFTY1/2 and CRIPTO decrease the proliferation of triple negative breast cancer cells. However, the combination of both LEFTY1/2 and CRIPTO together increase the proliferation of hormone responsive breast cancer cells. This effect is dependent on the ratio of the two proteins, observed by the effect at 50 ng/ml and not at higher concentrations of the two proteins.

FIGS. 17A and 17B show that 1189 IMM (also referred as Lefty IMM) and 1189 SCR1 (also referred as Lefty SCR) constructs are able to produce Lefty protein in culture. SDS-Page analysis of Lefty protein expression from 1189 IMM and 1189 SCR1 antigens. FIG. 17A shows 4-20% denaturing, reducing and non-reducing, SDS-PAGE analysis of 1189 IMM. Lane 1 refers to molecular weight marker PageRuler (Thermo Fisher) shown in kilodaltons; Lane 2 refers to 1189 IMM in reducing condition; Lane 3 refers to blank lane; Lane 4 refers to 1189 IMM in non-reducing condition. FIG. 17B shows 4-20% denaturing, reducing and non-reducing, SDS-PAGE analysis of 1189 SCR1. Lane 1 refers to molecular weight marker PageRuler (Thermo Fisher) shown in kilodaltons; Lane 2 refers to 1189 SCR1 in reducing condition; Lane 3 refers to blank lane; Lane 4 refers to 1189 SCR1 in non-reducing condition.

FIG. 18 shows TGFb1 (10 ng/ml) and Activin A (500 ng/ml) decrease MCF7 cell line proliferation, an effect that is rescued by the vactosertib (EW7197, an ALK5/ALK2/ALK4 inhibitor), but not with Lefty R&D (rhLefty2 protein, R&D Systems, cat no, Lefty Imm (human Lefty 1-Rabbit Fc fusion protein), or Lefty Scr (human Lefty1-Human Fc fusion protein). These data show that human LEFTY1 and LEFTY2 proteins do not promote hormone responsive breast cancer cell proliferation by counteracting TGFB1 or Activin A signalling through ALK5, AL2 or ALK4.

FIG. 19 shows TGFb1 (10 ng/ml) and Activin A (500 ng/ml) decrease MDA-MB-231 cell line proliferation, an effect that is rescued by the vactosertib (EW7197, an ALK5/ALK2/ALK4 inhibitor), but not with Lefty R&D (rhLefty2 protein, R&D Systems, Lefty Imm (human Lefty1-Rabbit Fc fusion protein), or Lefty Scr (human Lefty1-Human Fc fusion protein). These data show that human LEFTY1 and LEFTY2 proteins do not promote triple negative breast cancer cell proliferation by counteracting TGFB1 or Activin A signalling through ALK5, AL2 or ALK4.

FIG. 20 shows the amino acid sequence of the human left-right determination factor 1 LEFTY1 (Homo Sapiens (human) sequence (UniProtKB-075610 (LFTY1_HUMAN); gene ID: 10637), was used to generate 3D models of the protein of interest and design recombinant protein antigens. FIG. 20 discloses SEQ ID NO: 32.

FIG. 21 Sequences of the designed LEFTY1 human protein antigens. DNA coding for the amino acid sequence of 1189 IMM, 1189 SCR1 and 1189 SCR2 were synthesised and cloned into the mammalian transient expression plasmid pETE V2. FIG. 21 discloses SEQ ID NOS 33-35, respectively, in order of appearance.

FIGS. 22A and 22B depict results of immunization campaigns in rabbits after several rounds of 1189 IMM antigen injection. The injection of 1189 IMM was able to generate an immune response and consequently antibodies able to recognized Lefty SCR antigen measured by ELISA.

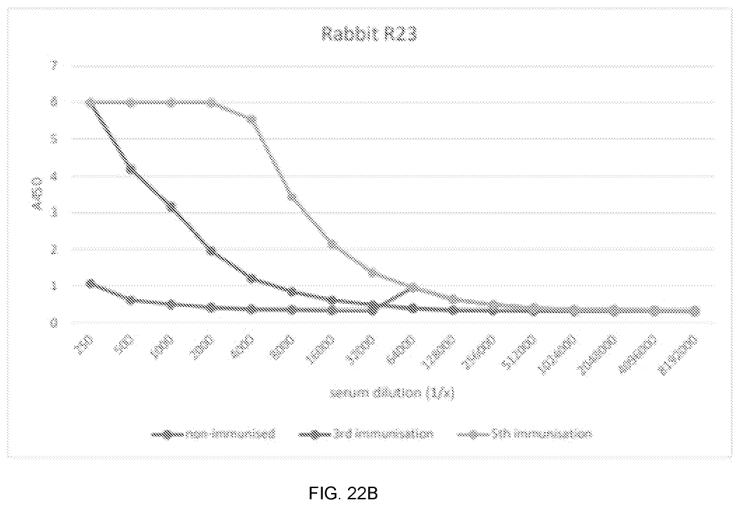

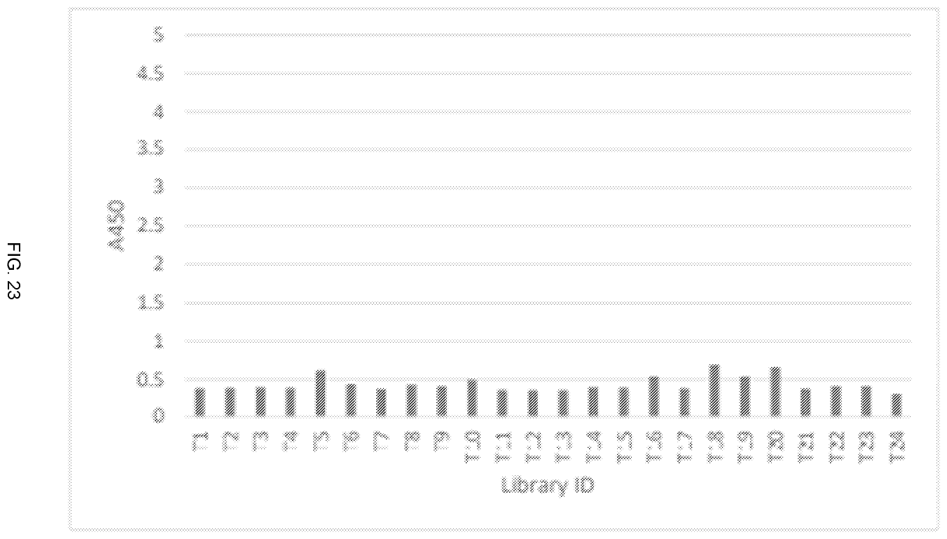

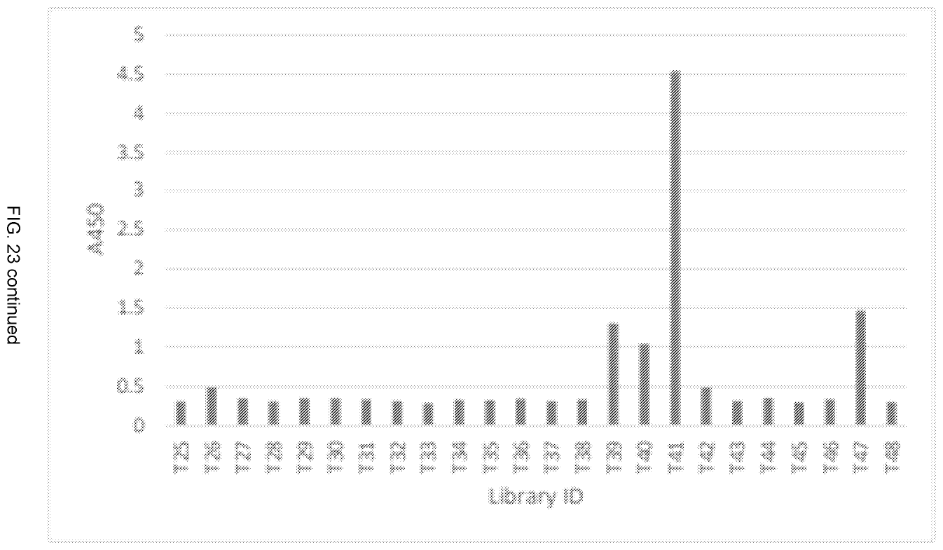

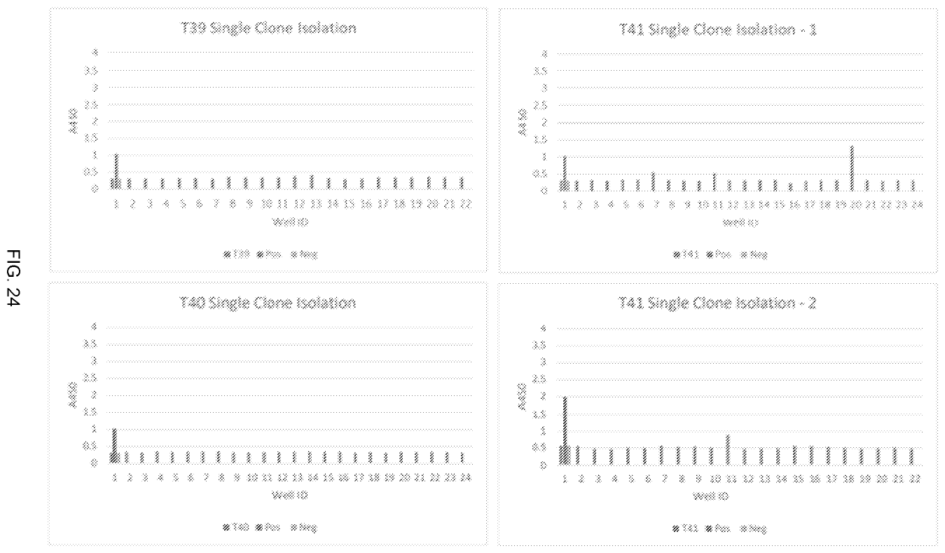

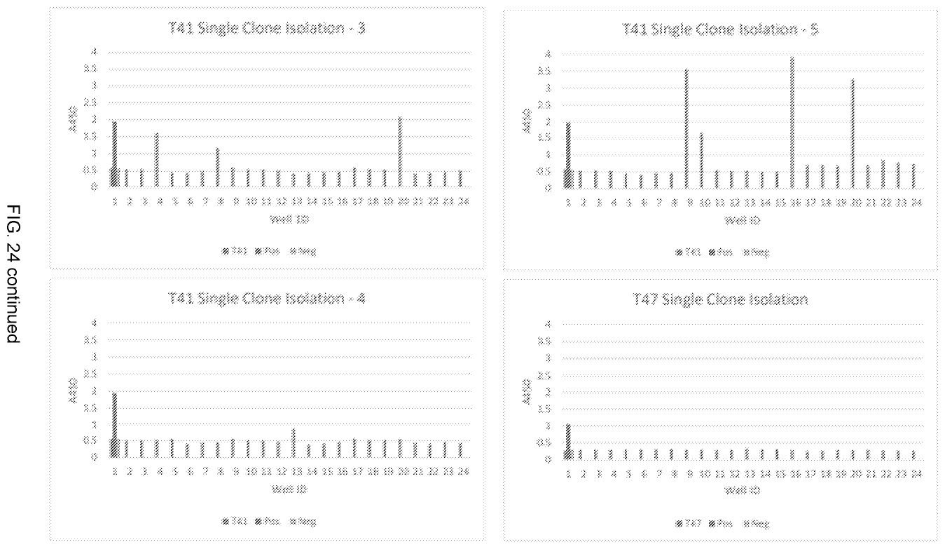

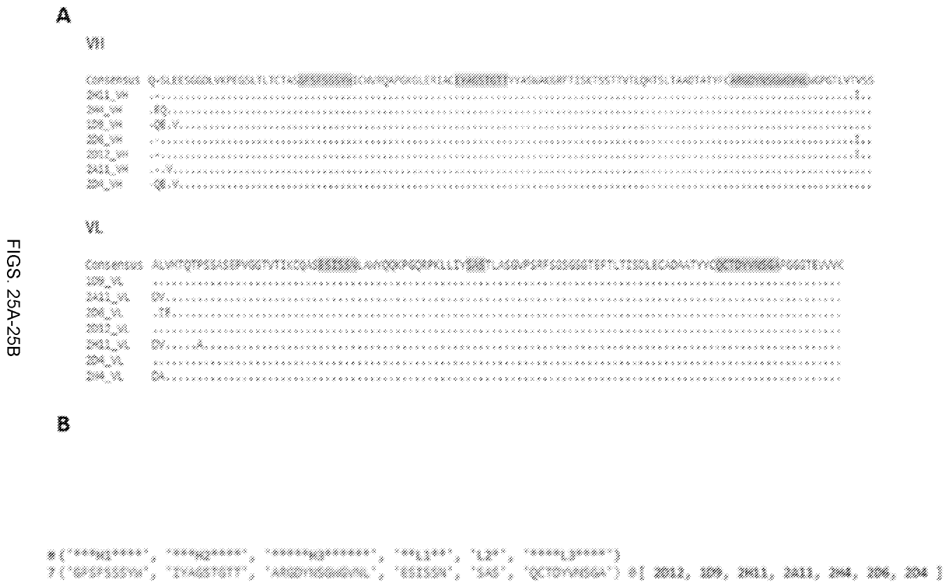

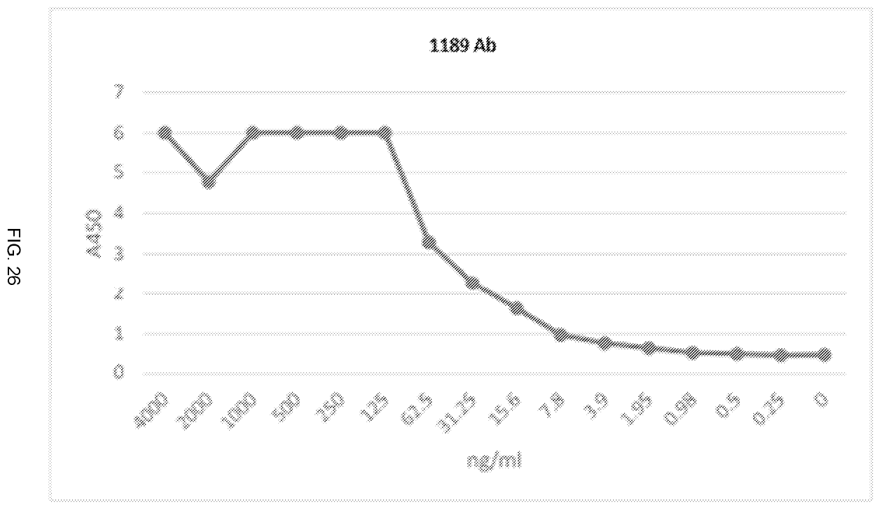

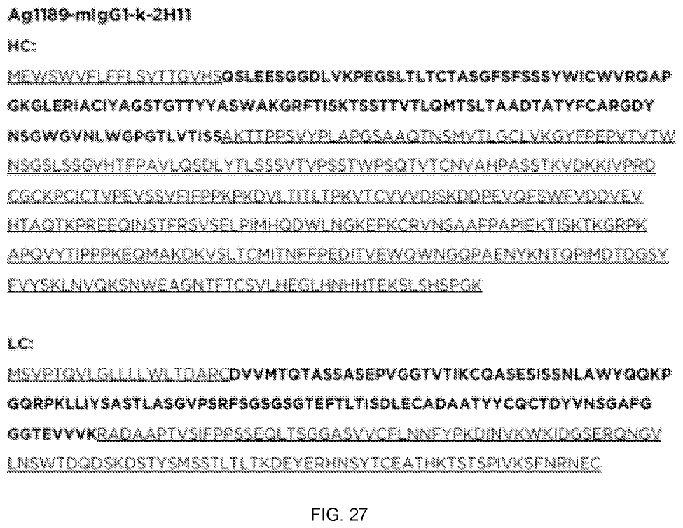

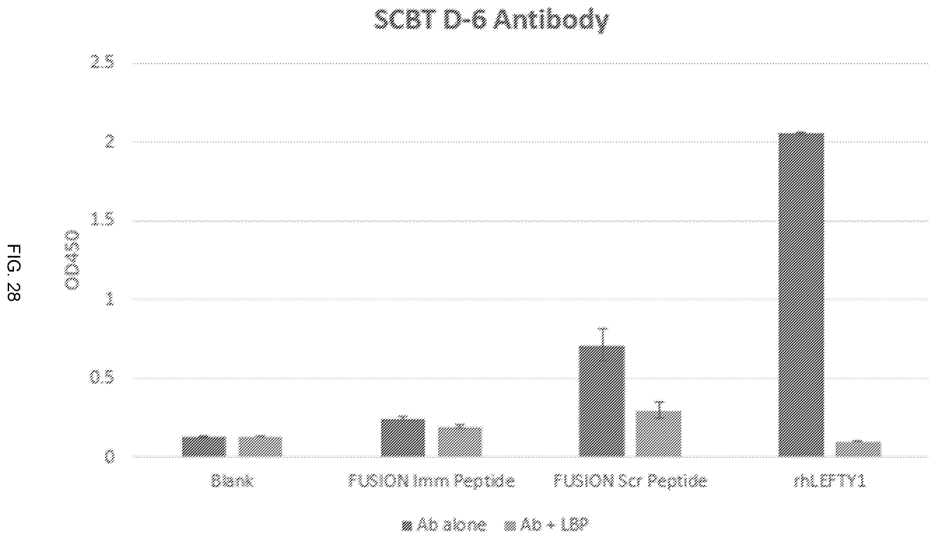

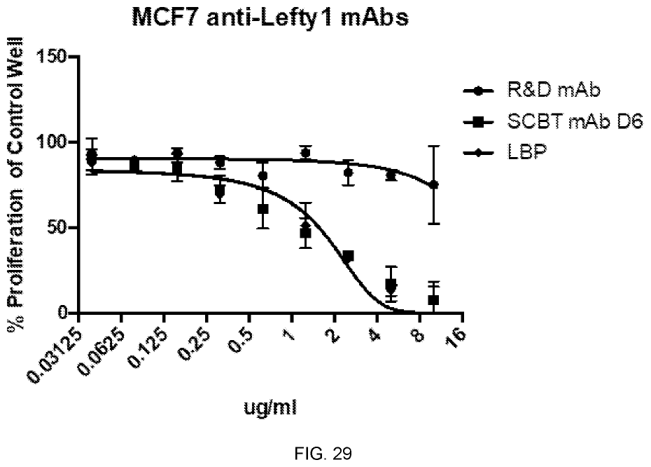

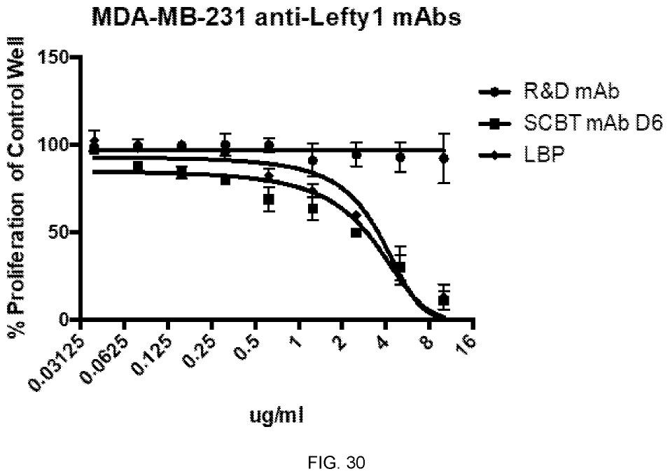

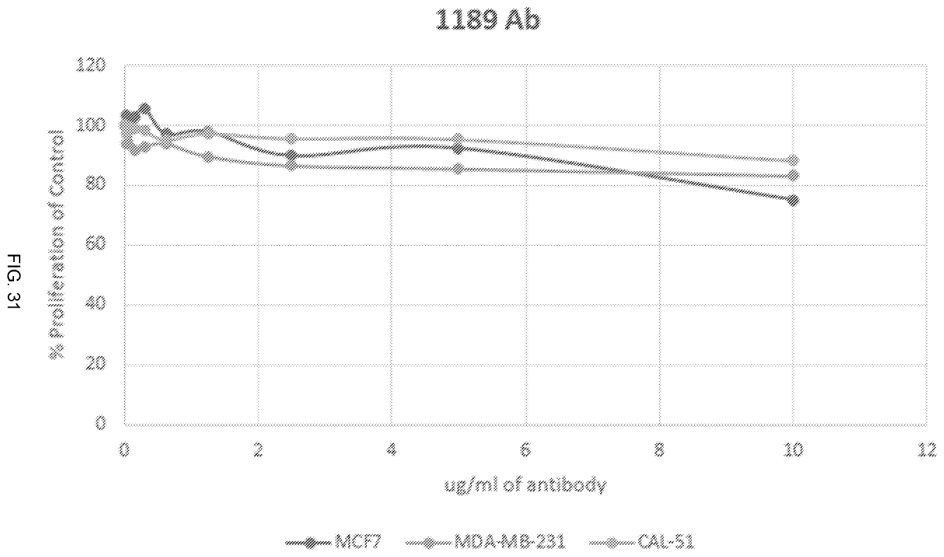

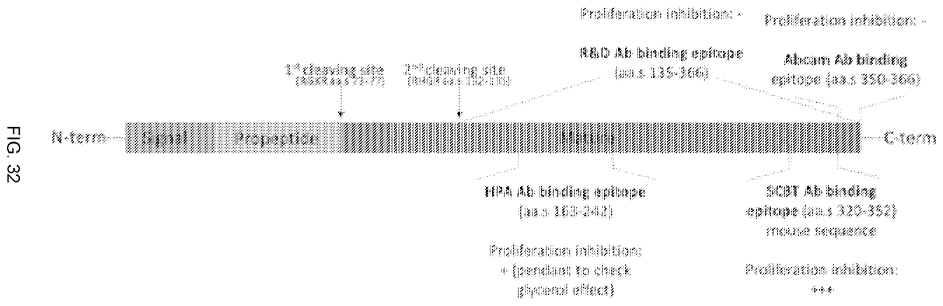

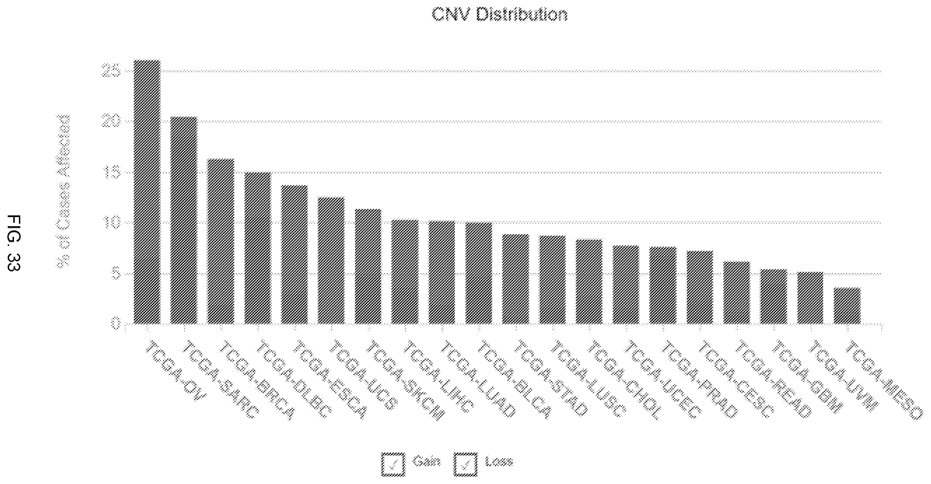

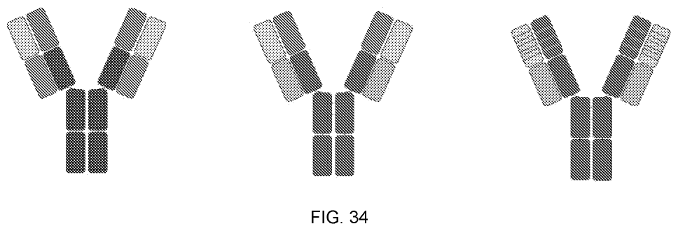

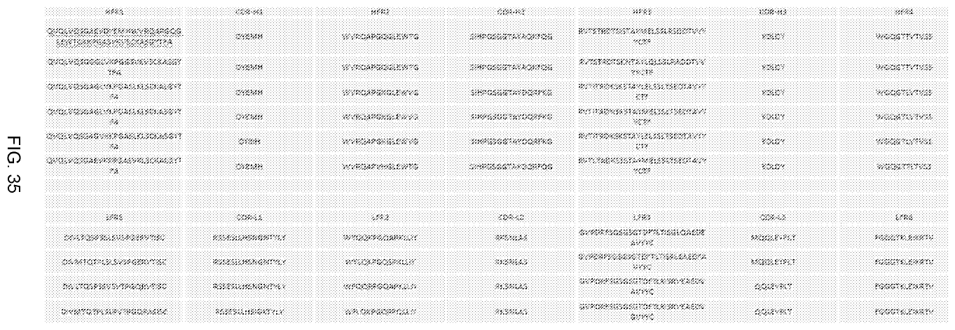

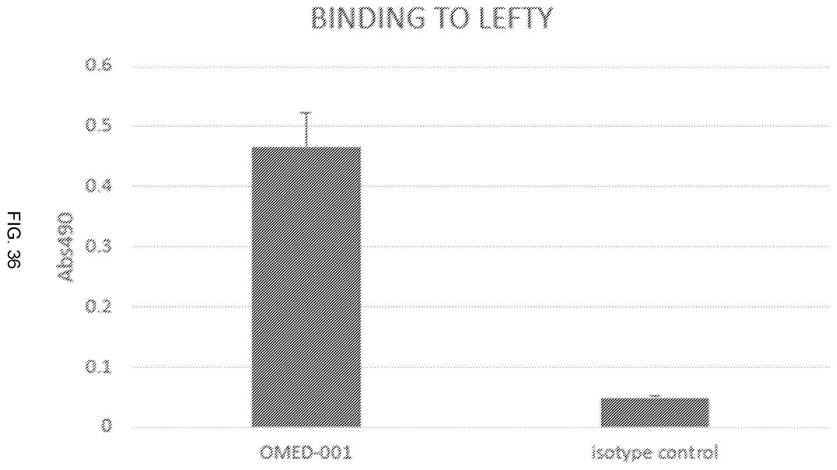

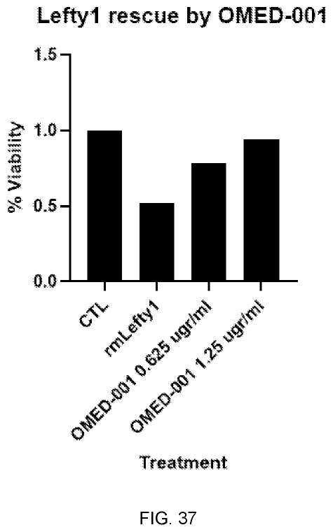

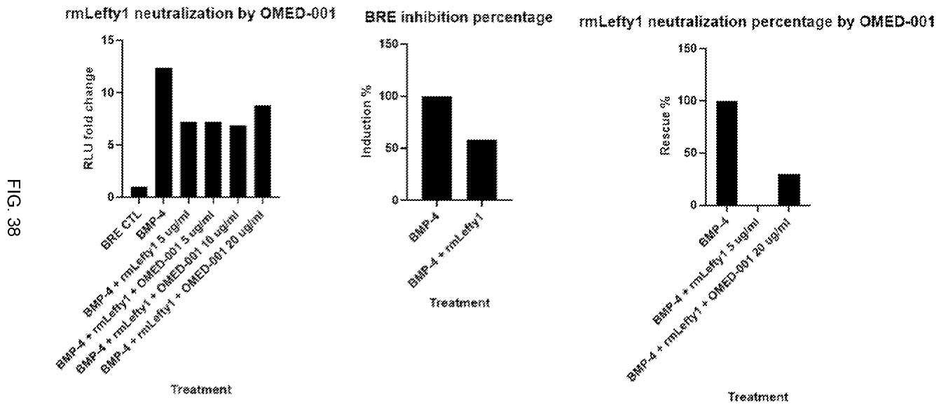

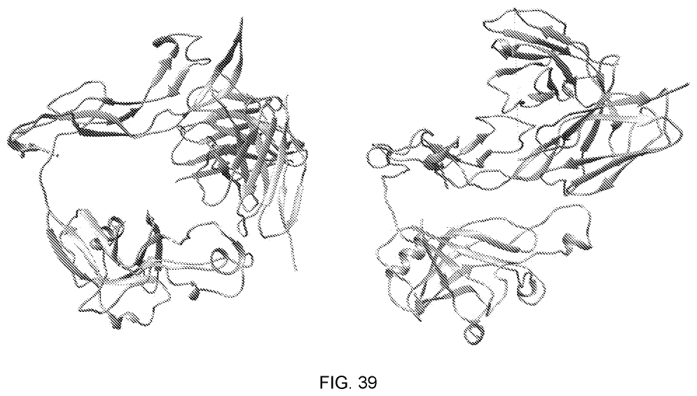

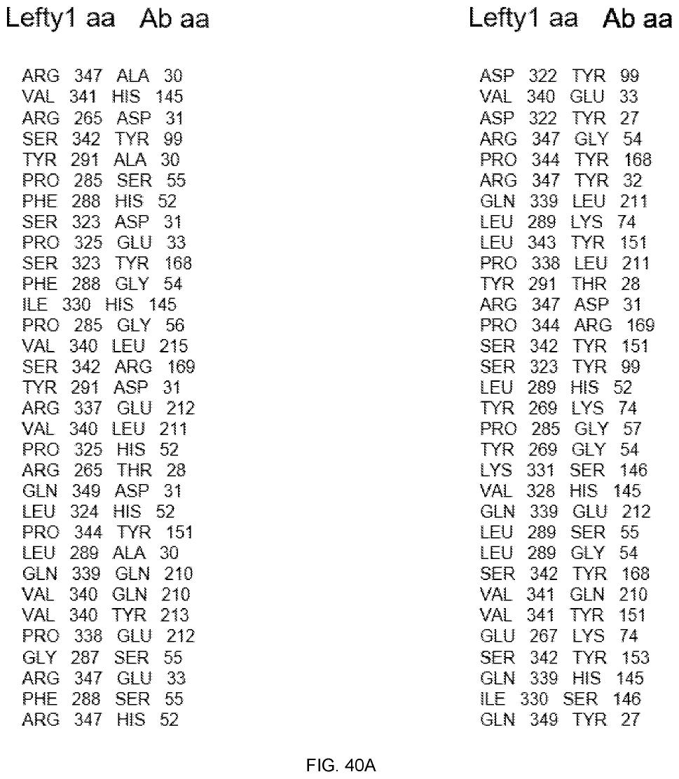

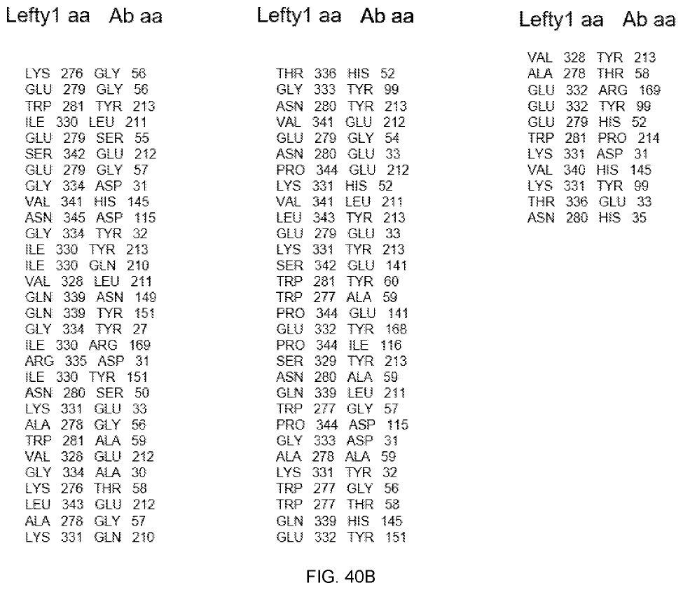

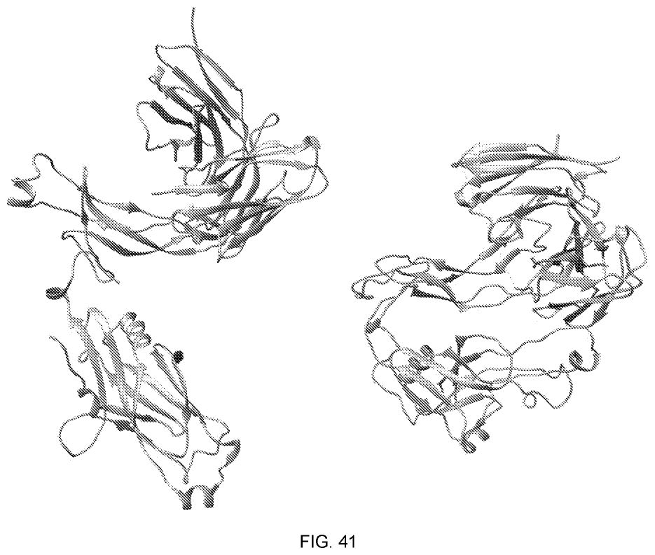

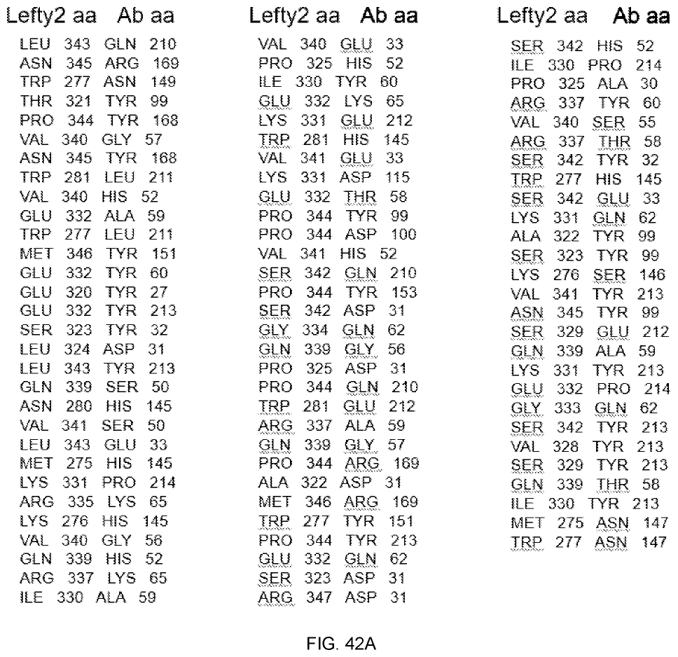

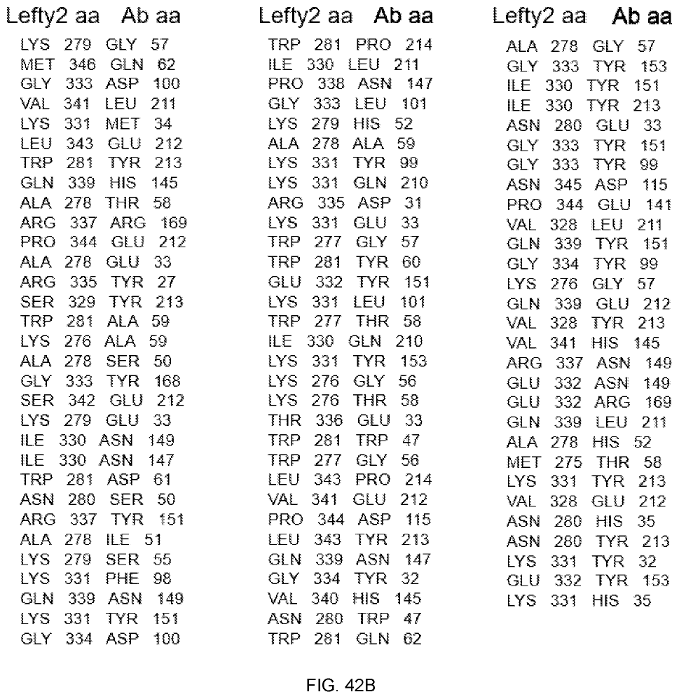

Two rabbits (R23 and R24) were immunized three times with 1189 IMM in three-week intervals (FIG. 22A). Animals were boosted three weeks after the third immunization before spleen isolation. The blood sera were obtained Day 0 and 10 days after second and third immunization. Immune response was tested from blood sera using ELISA screening against LEFTY1 SCR1. The immune response to LEFTY1 SCR after the third immunisations measured by ELISA is shown in FIG. 22A. The titer of rabbit R23 was calculated as 1:4000. Rabbit R24 didn't generate a response. Rabbit R23 received two additional 1189 IMM injections. Immune response tested from rabbit R23 sera after the fifth immunization was significantly increased with ELISA titer 1:32000 (FIG. 22B). Rabbit R23 was boosted three weeks after the fifth immunization, animal was sacrificed three days after final boost, splenocytes were isolated and stored in liquid nitrogen until use.

FIG. 23 shows ELISA data, testing of 48 mIgG1-k library pools developed from rabbit R23, absorvance at 450 nm. The injection of 1189 IMM was able to generate a set of family antibody pool libraries of VR and VH regions which were cloned into mIgG1-k vector.

FIG. 24 demostrates that the library pool of antibodies contained single clones able to produce antibodies against Lefty SCR antigen. The figure shows an ELISA response of single clones isolated from library pools to LEFTY1. Plasmid DNA from the four LEFTY1 specific ELISA positive pools was isolated and transfected into CHO cells for antibody transient production and subsequent analysis by ELISA on LEFTY1 SCR.

FIG. 25A-25B shows a variable region sequence alignment of an anti-LEFTY1 1189 antibody generated in the immunization campaing generated in rabbits. Eight mIgG1-k ELISA positive clones to LEFTY1 were identified in the libraries of antibodies. The aminoacidic sequence of those clones were analyzed by sequencing. A unique anti-LEFTY1 antibody was identified. FIG. 25A shows variable region sequence alignment. Antibodies are clustered, identical or similar VH and VL are grouped, and CDR-s are marked with blue in the consensus sequence (below). FIG. 25A discloses SEQ ID NOS 80, 36-38, 36, 36, 39, 38, 40, 40-42, 40, 43, 40 and 44, respectively, in order of appearance. FIG. 25B shows CDR-s of anti-LEFTY antibodies isolated from a rabbit. Heavy chain CDR-s are designed H1, H2, H3 and light CDR-s L1, L2, L3, respectively. Distance between individual unique CDR regions are shown in the distance matrix. FIG. 25B discloses SEQ ID NOS 1-5, respectively, in order of appearance.

FIG. 26 demonstrates that specific antibody clone is able to produce anti-LEFTY1 monoclonal antibody able to detect LEFTY SCR antigen. The figure shows an ELISA response of anti-LEFTY1 1189 specific mIgG1 antibody. The mIgG1 antibody clone 1 H11 was transfected into CHO cells for transient production in 6-well format. Produced antibody supernatant was tested by ELISA on LEFTY1 SCR coated plates (FIG. 26). The ELISA titer of this antibody to its binding to LEFTY1 SCR antigen is about 16 ng/ml.

FIG. 27 shows antibody heavy and light chain sequences of an anti-Ag1189-mIgG1-k-2H11 antibody. Heavy and light chain signal peptides and constant regions are underlines. Heavy and light chain variable regions are marked in bold. FIG. 27 discloses SEQ ID NOS 45-46, respectively, in order of appearance.

FIG. 28 shows an anti-LEFTY1 antibody binds to human LEFTY1-Rabbit Fc fusion protein, human LEFTY1-human Fc fusion protein, and rhLEFTY1 (recombinant human LEFTY2) protein. This effect is reduced when a Lefty1 peptide with sequence PMIV SVKEGGRTRPQVVSLPNMRVQT (SEQ ID NO: 14) is added. These data show that anti-LEFTY antibody that targets the C terminal 326-357 amino acids of mouse Lefty 1 binds to human LEFTY1 fusion proteins and recombinant human LEFTY2.

FIG. 29 shows anti-Lefty 1 antibodies and Lefty 1 peptide reduce the growth of MCF7 breast cancer cells. Lefty1 blocking peptide (LBP, derived from amino acids 327 to 352 at the C terminus of Lefty of mouse origin). Note that R&D system anti-Lefty 1 antibody (which binds from Leu136 to Pro368, Accession #Q64280.1) does not affect the proliferation of MCF7 cells, showing that only certain portions of Lefty1 are important for MCF7 breast cancer cell growth. These data show that anti-LEFTY antibodies the bind to and peptides derived from specific portions of Lefty1 protein sequence reduce the proliferation of hormone responsive breast cancer cells.

FIG. 30 shows anti-Lefty 1 antibodies and Lefty 1 peptide reduce the growth of MDA-MB-231 breast cancer cells. Lefty1 blocking peptide (LBP, derived from amino acids 327 to 352 at the C terminus of Lefty of mouse origin). Note that R&D system anti-Lefty1 antibody (which binds from Leu136 to Pro368, Accession #Q64280.1) does not affect the proliferation of MCF7 cells, showing that only certain portions of Lefty1 are important for MCF7 breast cancer cell growth. These data show that anti-LEFTY antibodies the bind to and peptides derived from specific portions of Lefty1 protein sequence reduce the proliferation of triple negative breast cancer cells.

FIG. 31 shows anti-LEFTY1 1189 monoclonal Ab reduces the proliferation of MCF7, MDA-MB-231 and CAL51 breast cancer cells. These data show that anti-LEFTY monoclonal antibody reduces the proliferation of hormone responsive and triple negative breast cancer cells.

FIG. 32 shows anti-LEFTY antibodies have different abilities to reduce the proliferation of breast cancer cells. C-terminal oriented antibodies reduce the proliferation of breast cancer cells that are dependent on LEFTY1 for their growth. The schematic represents the structure of LEFTY proteins in 2D, with annotations for the naturally occurring signal, propeptide, mature chain and proteolytic processing cleavage sites. The summary of the effect of different anti-LEFTY antibodies on the growth of MDA-MB-231 breast cancer cells are summarized, as well the region of LEFTY1 protein the are raised against. R&D Ab (R&D Systems, MAB994), Abcam Ab (Abcam, catalog number ab22569), SCBT Ab (Santa Cruz Biotechnology, catalog number sc-36584), HPA Ab (Human Protein Atlas, catalog number HPA056210).

FIG. 33 shows human LEFTY1 is copy number amplified in human cancers, making these cancers target indications for an anti LEFTY1 antibody treatment. The TCGA (The Cancer Genome Atlas.) database was searched for cancer that have either a gain of copy number (in red) or loss of copy number (blue) across datasets of different human cancers. Abbreviations: CNV (copy number variation, TCGA, The Cancer Genome Atlas; OV, Ovarian serous cystadenocarcinoma; SARC, sarcoma; BRCA; Breast invasive carcinoma DLBC, Lymphoid Neoplasm Diffuse Large B-cell Lymphoma; ESCA, Esophageal carcinoma; UCS, Uterine Carcinosarcoma; SKCM, Skin Cutaneous Melanoma; LIHC, Liver hepatocellular carcinoma; LUAD, Lung adenocarcinoma; BLCA, Bladder Urothelial Carcinoma; STAD, Stomach adenocarcinoma; LUSC, Lung squamous cell carcinoma; CHOL, Cholangiocarcinoma; UCEC, Uterine Corpus Endometrial Carcinoma; PRAD, Prostate adenocarcinoma; CESC, Cervical squamous cell carcinoma and endocervical adenocarcinoma; READ, Rectum adenocarcinoma; GBM, Glioblastoma multiforme; UVM, Uveal Melanoma, MESO, Mesothelioma.

FIG. 34 depicts structures of the SCBT antibody, OMED-001 and humanized candidates. The SCBT antibody depicts mouse kappa and mouse IgG1 regions. OMED-001 depicts mouse variable regions, human kappa and human IgG1 regions. Humanized candidates depict mouse CDRs grafted on human germlines of closest homology regions, human kappa and human IgG1 regions.

FIG. 35 presents the sequence of humanized candidates: 6 heavy chains and 4 light chains, which were done by CDR grafting into IGHV1-2 and IGKV2D-29 respectively followed by back mutations in residues within the vernier zone. FIG. 35 discloses 47-56, 6, 6, 6, 6-7, 6, 10, 10, 10-11, 57, 57-58, 58, 58-63, 8, 8, 64, 64, 64-65, 12, 12, 12, 12, 66-69, 68, 70-74, 9, 9, 9, 9, 9, 9, 13, 13, 75, 75-76, 76-77, 77, 77-79, 79, 79 and 79, respectively, top to bottom, in order of appearance.

FIG. 36 presents the binding of OMED-001 to immobilized Lefty protein analyzed by ELISA. Purified OMED-001 or isotype control antibody at 100 ng/ml was incubated with immobilized lefty followed by colorimetric detection.

FIG. 37 depicts Lefty 1 rescue by OMED-001.

FIG. 38 depicts BRE. BMP4 inhibition neutralization by Omed001.

FIG. 39 depicts the best docking conformation of the Ab onto Lefty1 in its open and compact conformations.

FIG. 40A depicts a map of contacts between the Ab and the OPEN Lefty1 conformation. The Lefty 1 aa involved in the docking are: 265, 267, 269, 285, 287, 288, 289, 291, 322, 323, 324, 325, 328, 330, 331, 337, 338, 339, 340, 341, 342, 343, 344, 347, 349. Predicted binding affinity (kcal·mol−1): −12.5. Predicted dissociation constant (M) at 25.0° C.: 6.8e-10

FIG. 40B depicts a map of contacts between the Ab and the COMPACT Lefty1 conformation. The Lefty 1 aa involved in the docking are: 276, 277, 278, 279, 280, 281, 328, 329, 330, 331, 332, 333, 334, 335, 336, 339, 340, 341, 342, 343, 344, 345. Predicted binding affinity (kcal·mol−1): −10.6. Predicted dissociation constant (M) at 25.0° C.: 1.8e-08.

FIG. 41 depicts a best docking conformation of the Ab onto Lefty2 in its open and compact conformations.

FIG. 42A depicts a map of contacts between the Ab and the OPEN Lefty2 conformation. The Lefty2 aa involved in the docking are: 275, 276, 277, 280, 281, 320, 321, 322, 323, 324, 325, 328, 329, 330, 331, 332, 333, 334, 335, 337, 339, 340, 341, 342, 343, 344, 345, 346, 347. Predicted binding affinity (kcal·mol−1): −11.9. Predicted dissociation constant (M) at 25.0° C.: 1.9e-09.

FIG. 42B depicts a map of contacts between the Ab and the COMPACT Lefty2 conformation. The Lefty2 aa involved in the docking are: 275, 276, 277, 278, 279, 280, 281, 328, 329, 330, 331, 332, 333, 334, 335, 336, 337, 338, 339, 340, 341, 342, 343, 344, 345, 346. Predicted binding affinity (kcal·mol−1): −12.7. Predicted dissociation constant (M) at 25.0° C.: 4.5c-10.

### DETAILED DESCRIPTION OF THE INVENTION

Unless specifically defined otherwise, all technical and scientific terms used herein shall be taken to have the same meaning as commonly understood by one of ordinary skill in the art (e.g., molecular biology, biochemistry, antibodies, antibody fragments such as single chain fragment variable and clinical studies).

The term “immunoglobulin” will be understood to include any protein comprising an immunoglobulin domain. Exemplary immunoglobulins are antibodies. Additional proteins encompassed by the term “immunoglobulin” include domain antibodies, camelid antibodies and antibodies from cartilaginous fish (i.e., immunoglobulin new antigen receptors (IgNARs)). Generally, camelid antibodies and IgNARs comprise a V_{H}, however lack a V_{L} and are often referred to as heavy chain immunoglobulins. Other “immunoglobulins” include T cell receptors.

The term “antibody” is used in the context of the present disclosure to refer to immunoglobulin molecules immunologically reactive with a particular antigen and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)2. Fab, Fv and rlgG as discussed in Pierce Catalogue and Handbook, 1994-1995 (Pierce Chemical Co., Rockford, Ill.); Kuby, J., Immunology, 3.sup.rd Ed., W.H. Freeman & Co., New York (1998). The term is also used to refer to recombinant single chain Fv fragments (scFv) as well as divalent (di-scFv) and trivalent (tri-scFV) forms thereof. The term antibody also includes bivalent or bispecific molecules, diabodies, triabodies, and tetrabodies. Examples of bivalent and bispecific molecules are described in Kostelny et al. (1992) J Immunol 148:1547; Pack and Pluckthun (1992) Biochemistry 31:1579; Hollinger et al., 1993, supra, Gruber et al. (1994) J. Immunol.: 5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

An “antigen binding fragment” of an antibody comprises one or more variable regions of an intact antibody. Examples of antibody fragments include Fab, Fab′, F(ab′)2 and Fv fragments; diabodies; linear antibodies; single-chain antibody molecules and multispecific antibodies formed from antibody fragments. For example, the term antigen binding fragment may be used to refer to recombinant single chain Fv fragments (scFv) as well as divalent (di-scFv) and trivalent (tri-scFV) forms thereof. Such fragments can be produced via various methods known in the art.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region typically has three CDR regions identified as CDR1, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system” or “Kabat”.

Other conventions that include corrections or alternate numbering systems for variable domains include IMGT (Lefranc, et al. (2003), Dev Comp Immunol 27:55-77), Chothia (Chothia C, Lesk A M (1987), J Mal Biol 196:901-917; Chothia, et al. (1989), Nature 342:877-883) and AHo (Honegger A, Pluckthun A (2001) J Mol Biol 309:657-670).

“Framework regions” (Syn. FR) are those variable domain residues other than the CDR residues.

The term “constant region” as used herein, refers to a portion of heavy chain or light chain of an antibody other than the variable region. In a heavy chain, the constant region generally comprises a plurality of constant domains and a hinge region, e.g., a IgG constant region comprises the following linked components, a constant heavy C_{H1}, a linker, a C_{H2} and a C_{H3}. In a heavy chain, a constant region comprises a Fc. In a light chain, a constant region generally comprise one constant domain (a CL1).

The term “fragment crystalizable” or “Fc” or “Fc region” or “Fc portion” (which can be used interchangeably herein) refers to a region of an antibody comprising at least one constant domain and which is generally (though not necessarily) glycosylated and which is capable of binding to one or more Fc receptors and/or components of the complement cascade. The heavy chain constant region can be selected from any of the five isotypes: alpha, delta, epsilon, gamma, or mu. Exemplary heavy chain constant regions are gamma 1 (IgG1), gamma 2 (IgG2) and gamma 3 (IgG3), or hybrids thereof.

A “constant domain” is a domain in an antibody the sequence of which is highly similar in antibodies/antibodies of the same type, e.g., IgG or IgM or IgE. A constant region of an antibody generally comprises a plurality of constant domains, e.g., the constant region of gamma, alpha or delta heavy chain comprises two constant domains.

The term “naked” is used to refer to antibodies and antigen binding fragments thereof of the present disclosure that are not conjugated to another compound, e.g., a toxic compound or radiolabel. For example, the term “naked” can be used to refer to antibodies and antigen binding fragments thereof such as di-scFv that are not conjugated to another compound. Accordingly, in one example, the antibodies and antigen binding fragments thereof of the present disclosure are “naked”. Put another way, antibodies and antigen binding fragments thereof of the present disclosure can be un-conjugated.

In contrast, the term “conjugated” is used in the context of the present disclosure to refer to antibodies or antigen binding fragments thereof of the present disclosure that are conjugated to another compound, e.g., a toxic compound such as a cytotoxic agent or radiolabel. Accordingly, in one example, the antibodies or antigen binding fragments thereof of the present disclosure are “conjugated”.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, S₁₅₃, Bi, P, Pb and radioactive isotopes of Lu), chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and antitumor or anticancer agents.

Terms such as “host cell,” “host cell line,” and “host cell culture” are used interchangeably in the context of the present disclosure to refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

An “isolated nucleic acid” according to the present disclosure is a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill of those practicing in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, the term “binds” in reference to the interaction of an antibody and antigen binding fragment thereof and an antigen means that the interaction is dependent upon the presence of a particular structure (e.g., an antigenic determinant or epitope) on the antigen. For example, an antibody and antigen binding fragment thereof recognizes and binds to a specific antigen structure rather than to antigens generally. For example, if an antibody and antigen binding fragment thereof binds to epitope “A”, the presence of a molecule containing epitope “A” (or free, unlabeled “A”), in a reaction containing labeled “A” and the antibody or antigen binding fragment thereof, will reduce the amount of labeled “A” bound to the antibody and antigen binding fragment thereof.

As used herein, the term “specifically binds” shall be taken to mean that the binding interaction between an antibody or antigen binding fragment thereof and an antigen is dependent on detection of the antigen by the antibody or antigen binding fragment thereof. Accordingly, the antibody or antigen binding fragment thereof preferentially binds or recognizes the antigen even when present in a mixture of other molecules or organisms. “Specific binding” does not necessarily require exclusive binding or non-detectable binding of another antigen. The term “specifically binds” can be used interchangeably with “selectively binds” herein. Generally, reference herein to binding means specific binding, and each term shall be understood to provide explicit support for the other term. Methods for determining specific binding will be apparent to the skilled person.

Antibodies or antigen binding fragments thereof according to the present disclosure and compositions comprising the same can be administered to a subject to treat various indications. Terms such as “subject”, “patient” or “individual” are terms that can, in context, be used interchangeably in the present disclosure. In an example, the subject is a mammal. The mammal may be a companion animal such as a dog or cat, or a livestock animal such as a horse or cow. In one example, the subject is a human. For example, the subject can be an adult. In another example, the subject can be a child. In another example, the subject can be an adolescent.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with a disease are mitigated or eliminated.

As used herein, the term “prevention” includes providing prophylaxis with respect to occurrence or recurrence of a disease in an individual. An individual may be predisposed to or at risk of developing the disease or disease relapse but has not yet been diagnosed with the disease or the relapse.

The term “treatment” is used in the context of the present specification to refer to the medical management of a patient with the intent to cure, ameliorate or stabilize a disease, pathological condition, or disorder. The term “treatment” includes active treatment, that is, treatment directed specifically toward the improvement of a disease, pathological condition, or disorder, and also includes causal treatment, that is, treatment directed toward removal of the cause of the associated disease, pathological condition, or disorder. In addition, the term “treatment” includes palliative treatment, that is, treatment designed for the relief of symptoms rather than the curing of the disease, pathological condition, or disorder; prophylactic treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

An “effective amount” refers to at least an amount effective, at dosages and for periods of time necessary, to achieve the desired therapeutic or prophylactic result. An effective amount can be provided in one or more administrations. In some examples of the present disclosure, the term “effective amount” is meant an amount necessary to effect treatment of a disease or condition described below. The effective amount may vary according to the disease or condition to be treated and also according to the weight, age, racial background, sex, health and/or physical condition and other factors relevant to the subject being treated. Typically, the effective amount will fall within a relatively broad range (e.g. a “dosage” range) that can be determined through routine trial and experimentation by a medical practitioner. The effective amount can be administered in a single dose or in a dose repeated once or several times over a treatment period.

A “therapeutically effective amount” is at least the minimum concentration required to effect a measurable improvement of a particular disorder (e.g. cancer). A therapeutically effective amount herein may vary according to factors such as the disease state, age, sex, and weight of the patient, and the ability of the antibody and antigen binding fragment thereof to elicit a desired response in the individual. A therapeutically effective amount is also one in which any toxic or detrimental effects of the antibody and antigen binding fragment thereof are outweighed by the therapeutically beneficial effects. In the case of cancer, the therapeutically effective amount of the antibody and antigen binding fragment thereof may reduce the number of cancer cells; reduce the primary tumor size; inhibit (i.e., slow to some extent and, in some examples, stop) cancer cell infiltration into peripheral organs; inhibit (i.e., slow to some extent and, in some examples, stop) tumor metastasis; inhibit or delay, to some extent, tumor growth or tumor progression; and/or relieve to some extent one or more of the symptoms associated with the cancer. To the extent the antibody and antigen binding fragment thereof may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

Monoclonal antibodies are one exemplary form of antibody and antigen binding fragment thereof contemplated by the present disclosure. The term “monoclonal antibody” or “MAb” refers to a homogeneous antibody population capable of binding to the same antigen(s), for example, to the same epitope within the antigen. This term is not intended to be limited as regards to the source of the antibody or the manner in which it is made.

In an example, antibodies and antigen binding fragments thereof encompassed by the present disclosure may be “humanized”. A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. In an example, the humanized antibody will also comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)).

In an example, “human” antibodies and antigen binding fragments thereof of the present disclosure can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4 or 5 of the residues of the protein). These “human antibodies” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies.

In another example, antibodies and antigen binding fragments thereof encompassed by the present disclosure may be synhumanized. The term “synhumanized” refers to an antibody prepared by a method described in WO2007/019620. A synhumanized antibody includes a variable region of an antibody, wherein the variable region comprises FRs from a New World primate antibody variable region and CDRs from a non-New World primate antibody variable region.

In another example, an antibody and antigen binding fragment thereof of the present disclosure may be primatized. A “primatized antibody” comprises variable region(s) from an antibody generated following immunization of a non-human primate (e.g., a cynomolgus macaque). In an example, the variable regions of the non-human primate antibody are linked to human constant regions to produce a primatized antibody. Exemplary methods for producing primatized antibodies are described in U.S. Pat. No. 6,113,898.

In one example, an antibody and antigen binding fragment thereof of the disclosure is a chimeric antibody or fragment. The term “chimeric antibody” or “chimeric antigen binding fragment” refers to an antibody or fragment in which one or more of the variable domains is from a particular species (e.g., murine, such as mouse or rat) or belonging to a particular antibody class or subclass, while the remainder of the antibody or fragment is from another species (such as, for example, human or non-human primate) or belonging to another antibody class or subclass. In one example, a chimeric antibody comprising a VH and/or a VL from a non-human antibody (e.g., a murine antibody) and the remaining regions of the antibody are from a human antibody.

The present disclosure also contemplates a deimmunized antibody or antigen binding fragment thereof, e.g., as described in WO2000/34317 and WO2004/108158. De-immunized antibodies and fragments have one or more epitopes, e.g., B cell epitopes or T cell epitopes removed (i.e., mutated) to thereby reduce the likelihood that a subject will raise an immune response against the antibody or protein. For example, an antibody of the disclosure is analyzed to identify one or more B or T cell epitopes and one or more amino acid residues within the epitope is mutated to thereby reduce the immunogenicity of the antibody.

In some examples, an antibody and antigen binding fragment thereof of the disclosure is or comprises a single-domain antibody (which is used interchangeably with the term “domain antibody” or “dAb”). A single-domain antibody is a single polypeptide chain comprising all or a portion of the heavy chain variable domain of an antibody.

One of skill in the art will be aware that scFv's comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). Single-chain variable fragments lack the constant Fc region found in complete antibody molecules and therefore can have reduced immunogenicity. Exemplary linkers comprise in excess of 12 amino acid residues with (Gly₄Ser)₃ (SEQ ID NO: 15) being one of the more favoured linkers for a scFv.

The present disclosure also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

In another example, the present disclosure encompasses a dimeric scFv (di-scFV), i.e., a protein comprising two scFv molecules linked by a non-covalent or covalent linkage, e.g., by a leucine zipper domain (e.g., derived from Fos or Jun) or trimeric scFV (tri-scFv). In another example, two scFv's are linked by a peptide linker of sufficient length to permit both scFv's to form and to bind to an antigen, e.g., as described in U.S. Published application No. 20060263367.

In some examples, an antigen binding fragment of the disclosure is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fv's having different specificity).

The present invention encompasses modifying the Fc portion of antibodies to confer neutralizing properties. Mutating the Fc region allows either triggering or suppressing blood complement activation to kill cells that are producing LEFTY proteins. Also, engineering the Fc region is a method to alter the biophysical properties of a LEFTY neutralizing antibody (e.g. serum half life, volume of distribution in the body, thermostability) which is unique to the antibody. Methods for modifying the Fc portion of antibodies to confer neutralizing properties is known to one of skill the art and is reviewed, for example, in Saunders, Front. Immunol., 7 Jun. 2019 | https://doi.org/10.3389/fimmu.2019.01296 and described by Kang & Jung Experimental & Molecular Medicine volume 51, pages 1-9 (2019).

The present invention relates to an antibody or antigen binding fragment thereof that binds to a LEFTY protein, such as LEFTY1, and inhibits the growth of cancer cells, such as breast cancer cells.

In an advantageous embodiment, the antibody or antigen binding fragment thereof, may comprise a heavy chain variable region and a light chain variable region, wherein the heavy chain variable region may comprise heavy chain contemplentarity determining regions (CDRs) comprising the amino acid sequences GFSFSSSYW (SEQ ID NO: 1), IYAGSTGTT (SEQ ID NO: 2) and ARGDYNSGWGVNL (SEQ ID NO: 3), and wherein the light chain variable region may comprise light chain CDRs comprising the amino acid sequences of ESISSN (SEQ ID NO: 4), SAS and QCTDYVNSGA (SEQ ID NO: 5).

In another embodiment, present invention relates to an antibody or antigen binding fragment thereof, which may comprise a heavy chain variable region and a light chain variable region, wherein the heavy chain variable region may comprise the amino acid sequences DYEMH (SEQ ID NO: 6) or DYEIH (SEQ ID NO: 7), SIHPGSGGTAYAQKFQG (SEQ ID NO: 8) and YDLDY (SEQ ID NO: 9), and wherein the light chain variable region may comprise light chain CDRs comprising the amino acid sequences of RSSESLLHSNGNTYLY (SEQ ID NO: 10) or RSSESLLHSIGKTYLY (SEQ ID NO: 11), RKSNLAS (SEQ ID NO: 12) and MQQLEYPLT (SEQ ID NO: 13).

In another advantageous embodiment, the antibody or antigen binding fragment thereof has at least 80%, at least 82%, at least 85%, at least 87%, at least 90%, at least 91%, at least 92%, at least 93%, at least 94%, at least 95%, at least 96%, at least 97%, at least 98%, at least 99% or 100% sequence identity of any of the variable heavy chain and variable light chain sequences as shown in FIG. 25A.

In another advantageous embodiment, the antibody or antigen binding fragment thereof has at least 80%, at least 82%, at least 85%, at least 87%, at least 90%, at least 91%, at least 92%, at least 93%, at least 94%, at least 95%, at least 96%, at least 97%, at least 98%, at least 99% or 100% sequence identity of any of the heavy chain or light chain sequences as shown in FIG. 27.

In another advantageous embodiment, the antibody or antigen binding fragment thereof has at least 80%, at least 82%, at least 85%, at least 87%, at least 90%, at least 91%, at least 92%, at least 93%, at least 94%, at least 95%, at least 96%, at least 97%, at least 98%, at least 99% or 100% sequence identity of any of the heavy chain or light chain sequences as shown in FIG. 35.

The present invention also encompasses screening for additional antibodies or antigen binding fragments thereof that bind one or more LEFTY proteins. Applicants have data from commercially available anti-LEFTY1 antibodies that are able to either block or not block the proliferation of two different breast cancer cell lines to gain insight into which specific part of LEFTY1 is necessary to be blocked to reduce breast cancer cell growth. Therefore, the present invention relates to methods of identifying additional anti-LEFTY1 antibodies with specific regions of LEFTY1 identified by Applicants such as, but limited to the C-terminal region of LEFTY1 (see, e.g., FIG. 32). In particular, residues 320 to 366 of the LEFTY1 protein are preferred epitope regions. The present invention also encompasses antibodies or antigenic binding fragments thereof that specifically bind to epitopic regions of C-terminal region of LEFTY-1, such as but not limited to residues 320 to 366 of the LEFTY1 protein.

The present invention also encompasses the administration of antigenic regions of the LEFTY protein, such as the C-terminal region of the LEFTY1 protein or residues 320 to 366 of the LEFTY1 protein, which are encompassed by the term “LEFTY antigen”. The LEFTY antigen may be administered by any suitable method, for example parenterally, orally or topically. Preferably, however the antigen is administered by injection, for example intramuscular, intradermal, intravenous or subcutaneous injection, more preferably by subcutaneous or intravenous injection.

Adjuvants are any substance whose admixture with an administered antigen increases or otherwise modifies the immune response to said antigen. Adjuvants may for example be selected from the group consisting of AlK(SO₄)₂, AlN₄ (SO₄)₂. AlNH₄ (SO₄), silica, alum, Al(OH)₃, Cas (PO₄)₂, kaolin, carbon, aluminum hydroxide, muramyl dipeptides, N-acetyl-muramyl-L-thrconyl-D-isoglutamine (thr-DMP), N-acetyl-nornuramyl-L-alanyl-D-isoglutamine (CGP 11687, also referred to as nor-MDP), N-acetylmuramyul-L-alanyl-D-isoglutaminyl-L-alanine-2-(1′2′-dipalmitoyl-s-n-glycero-3-hydroxphosphoryloxy)-ethylamine (CGP 19835A, also referred to as MTP-PE), RIBI (MPL+TDM+CWS) in a 2% squalene/Tween-80® emulsion, lipopolysaccharides and its various derivatives, including lipid A, Freund's Complete Adjuvant (FCA), Freund's Incomplete Adjuvants, Merck Adjuvant 65, polynucleotides (for example, poly IC and poly AU acids), wax D from Mycobacterium, tuberculosis, substances found in Corynebacterium parvum, Bordetella pertussis, and members of the genus Brucella, liposomes or other lipid emulsions, Titermax, ISCOMS, Quil A, ALUN (see U.S. Pat. Nos. 58,767 and 5,554,372), Lipid A derivatives, choleratoxin derivatives, HSP derivatives, LPS derivatives, synthetic peptide matrixes or GMDP, Interleukin 1, Interleukin 2, Montanide ISA-51 and QS-21. Preferred adjuvants to be used with the invention include Freund's Complete Adjuvant (FCA), Freund's Incomplete Adjuvants.

Carriers are scaffold structures, e.g. a polypeptide or a polysaccharide, to which an antigen is capable of being associated. A carrier may be present independently of an adjuvant. The function of a carrier can for example be to increase the molecular weight of in particular the LEFTY antigen in order to increase the immunogenicity, to confer stability, to increase the biological activity, or to increase serum half-life. The carrier may be any suitable carrier known to the person skilled in the art, for example a protein or an antigen presenting cell. A carrier protein could be, but is not limited to keyhole limpet hemocyanin, serum proteins such as transferrin, bovine serum albumin, human serum albumin, thyroglobulin or ovalbumin, immunoglobulins, or hormones, such as insulin or palmitic acid.

The mammal may be any suitable mammal. Monoclonal antibodies are frequently prepared using a rodent, for example a mouse or a rat Polyclonal antibodies may be prepared by administering the LEFTY antigen to any mammal, for example mice, rats, rabbits, donkeys, goats, sheeps, cows or camels. Antibodies according to the invention may also be mixtures of antibodies, such as mixtures of monoclonal antibodies, mixtures of polyclonal antobodies or both. Hence, it is also comprised within the invention that more than one kind of animal may be used.

If the antibody is a monoclonal antibody, antibody producing cells are usually isolated from said mammal subsequent to immunization. The method may for example comprise the steps of isolating antibody producing cells from said mammal, preparing hybridoma cells from said antibody producing cells, cultivating said hybridomas and isolating antibodies produced by said hybridomas.

For example said cells may be isolated from said mammal 1 day, such as in the range of 2 to 10 days, for example in the range of 10 to 20 days, such as in the range of 20 to 40 days, for example in the range of 1 to 3 months, such as in the range of 3 to 6 months, for example in the range of 6 to 12 months, such as in the range of 12 to 24 months, for example more than 24 months after first administration of the LEFTY antigen.

The antibody producing cells are in general B-cells and said cells may for example be isolated from said mammal by excising the spleen of said mammal.

Once the antibody producing cells have been isolated from said mammal, the cells may be fused with other cells in order to obtain hybridoma cells. Said cells may for example be cancer cells, such as cells derived from a leukaemia, for example myeloma cells. After fusion said hybridoma cells may be cultivated using standard cultivation protocols.

The cultivation medium (supernatant) may be tested for the presence of suitable LEFTY antibodies and hybridoma cells capable of producing suitable LEFTY antibodies may be selected and cultivated.

Testing may be performed by any suitable method, for example methods detecting the presence of antibodies capable of associating with the LEFTY antigen. Such methods include, but are not limited to Western blotting, ELISA (Enzyme-Linked Immunosorbent Assay), dot-blotting or TRIFMA. In addition or alternatively, said cultivation medium may be tested for the presence of LEFTY antibodies capable of inhibiting LEFTY activity.

Once hybridoma cells capable of producing suitable LEFTY antibodies have been identified, said cells may be cultivated using any standard protocol and antibodies produced by said cells may be purified. Purification of antibodies may be done using any standard protocol, for example purification using anti-Ig antibodies, protein G or protein A.

If the antibody is a polyclonal antibody, said antibody may for example be purified directly from serum from a mammal, immunised with the LEFTY antigen. Purification may be done using any standard method, for example purification using anti-Ig antibodies, protein G or protein A.

Methods of preparing monoclonal antibodies, mixtures of monoclonal antibodies or polycloncal antibodies are for example described in Antibodies: A Laboratory Manual, By Ed Harlow and David Lane, Cold Spring Harbor Laboratory Press, 1988.

As known in the art, antibodies can come in different isotypes such as IgA, IgD, IgE, IgG, and IgM. In an example, antibodies encompassed by the present disclosure are IgG. In another example, antibodies encompassed by the present disclosure are IgM.

The antibody or antigen binding fragment thereof as described herein may be recombinant.

In the case of a recombinant peptide or polypeptide, nucleic acid encoding same can be cloned into expression vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce immunoglobulin or antibody protein.

Suitable molecular cloning techniques are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art. See U.S. Pat. No. 4,816,567 or U.S. Pat. No. 5,530,101.

The nucleotide sequences of the present invention may be inserted into “vectors.” The term “vector” is widely used and understood by those of skill in the art, and as used herein the term “vector” is used consistent with its meaning to those of skill in the art. For example, the term “vector” is commonly used by those skilled in the art to refer to a vehicle that allows or facilitates the transfer of nucleic acid molecules from one environment to another or that allows or facilitates the manipulation of a nucleic acid molecule.

Any vector that allows expression of the antibodies of the present invention may be used in accordance with the present invention. In certain embodiments, the antibodies of the present invention may be used in vitro (such as using cell-free expression systems) and/or in cultured cells grown in vitro in order to produce the encoded LEFTY antibodies, which may then be used for various applications such as in the production of proteinaceous vaccines. For such applications, any vector that allows expression of the antibodies in vitro and/or in cultured cells may be used.

For applications where it is desired that the antibodies be expressed in vivo, for example when the transgenes of the invention are used in DNA or DNA-containing vaccines, any vector that allows for the expression of the antibodies of the present invention and is safe for use in vivo may be used. In preferred embodiments the vectors used are safe for use in humans, mammals and/or laboratory animals.

Following isolation, the nucleic acid is inserted operably linked to a promoter in an expression construct or expression vector for further cloning (amplification of the DNA) or for expression in a cell-free system or in cells. Thus, another example of the disclosure provides an expression construct that comprises an isolated nucleic acid of the disclosure and one or more additional nucleotide sequences. Suitably, the expression construct is in the form of, or comprises genetic components of, a plasmid, bacteriophage, a cosmid, a yeast or bacterial artificial chromosome as are understood in the art. Expression constructs may be suitable for maintenance and propagation of the isolated nucleic acid in bacteria or other host cells, for manipulation by recombinant DNA technology and/or for expression of the nucleic acid or an antibody and antigen binding fragment thereof of the disclosure.

For the antibodies of the present invention to be expressed, the protein coding sequence should be “operably linked” to regulatory or nucleic acid control sequences that direct transcription and translation of the protein. As used herein, a coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” may be any nucleic acid element, such as, but not limited to promoters, enhancers. IRES, introns, and other elements described herein that direct the expression of a nucleic acid sequence or coding sequence that is operably linked thereto. The term “promoter” will be used herein to refer to a group of transcriptional control modules that are clustered around the initiation site for RNA polymerase II and that when operationally linked to the protein coding sequences of the invention lead to the expression of the encoded protein. The expression of the transgenes of the present invention may be under the control of a constitutive promoter or of an inducible promoter, which initiates transcription only when exposed to some particular external stimulus, such as, without limitation, antibiotics such as tetracycline, hormones such as ecdysone, or heavy metals. The promoter may also be specific to a particular cell-type, tissue or organ Many suitable promoters and enhancers are known in the art, and any such suitable promoter or enhancer may be used for expression of the transgenes of the invention. For example, suitable promoters and/or enhancers may be selected from the Eukaryotic Promoter Database (EPDB).

The vectors used in accordance with the present invention should typically be chosen such that they contain a suitable gene regulatory region, such as a promoter or enhancer, such that the antibodies of the invention may be expressed.

For example, when the aim is to express the antibodies of the invention in vitro, or in cultured cells, or in any prokaryotic or eukaryotic system for the purpose of producing the protein(s) encoded by that antibody, then any suitable vector may be used depending on the application. For example, plasmids, viral vectors, bacterial vectors, protozoal vectors, insect vectors, baculovirus expression vectors, yeast vectors, mammalian cell vectors, and the like, may be used. Suitable vectors may be selected by the skilled artisan taking into consideration the characteristics of the vector and the requirements for expressing the antibodies under the identified circumstances.

In an advantageous embodiment, IgG1 and Fab expression vectors may be utilized to reconstitute heavy and light chain constant regions if heavy and light chain genes of the antibodies of the present invention are cloned.

When the aim is to express the antibodies of the invention in vivo in a subject, for example in order to generate an immune response against a LEFTY antigen and/or protective immunity against LEFTY, expression vectors that are suitable for expression on that subject, and that are safe for use in vivo, should be chosen. For example, in some embodiments it may be desired to express the antibodies of the invention in a laboratory animal, such as for pre-clinical testing of the LEFTY immunogenic compositions and vaccines of the invention. In other embodiments, it will be desirable to express the antibodies of the invention in human subjects, such as in clinical trials and for actual clinical use of the immunogenic compositions and vaccine of the invention. Any vectors that are suitable for such uses may be employed, and it is well within the capabilities of the skilled artisan to select a suitable vector. In some embodiments it may be preferred that the vectors used for these in vivo applications are attenuated to vector from amplifying in the subject. For example, if plasmid vectors are used, preferably they will lack an origin of replication that functions in the subject so as to enhance safety for in vivo use in the subject. If viral vectors are used, preferably they are attenuated or replication-defective in the subject, again, so as to enhance safety for in vivo use in the subject.

In preferred embodiments of the present invention viral vectors are used. Viral expression vectors are well known to those skilled in the art and include, for example, viruses such as adenoviruses, adeno-associated viruses (AAV), alphaviruses, herpesviruses, retroviruses and poxviruses, including avipox viruses, attenuated poxviruses, vaccinia viruses, and particularly, the modified vaccinia Ankara virus (MVA; ATCC Accession No. VR-1566). Such viruses, when used as expression vectors are innately non-pathogenic in the selected subjects such as humans or have been modified to render them non-pathogenic in the selected subjects. For example, replication-defective adenoviruses and alphaviruses are well known and may be used as gene delivery vectors.

Many vectors for expression in cells are available. The vector components generally include, but are not limited to, one or more of the following: a signal sequence, a sequence encoding the antibody or antigen binding fragment thereof (e.g., derived from the information provided herein), an enhancer element, a promoter, and a transcription termination sequence. Exemplary signal sequences include prokaryotic secretion signals (e.g., pelB, alkaline phosphatase, penicillinase, Ipp, or heat-stable enterotoxin II), yeast secretion signals (e.g., invertase leader, .alpha. factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-.alpha. promoter (EF1), small nuclear RNA promoters (U1a and U1b),.alpha.-myosin heavy chain promoter, Simian virus 40 promoter (SV40), Rous sarcoma virus promoter (RSV), Adenovirus major late promoter, .beta.-actin promoter; hybrid regulatory element comprising a CMV enhancer/beta-actin promoter or an immunoglobulin or antibody promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Alternatively, methods which are well known to those skilled in the art may be used to construct expression vectors containing nucleic acid molecules that encode the polypeptide or homologs or derivatives thereof under appropriate transcriptional/translational control signals, for expression. These methods include in vitro recombinant DNA techniques, synthetic techniques and in vivo recombination/genetic recombination. See, for example, the techniques described in Maniatis et al., 1989.

The nucleotide sequences and vectors of the invention may be delivered to cells, for example if the aim is to express the LEFTY antigens in cells in order to produce and isolate the expressed proteins, such as from cells grown in culture. For expressing the antibodies in cells any suitable transfection, transformation, or gene delivery methods may be used. Such methods are well known by those skilled in the art, and one of skill in the art would readily be able to select a suitable method depending on the nature of the nucleotide sequences, vectors, and cell types used. For example, transfection, transformation, microinjection, infection, electroporation, lipofection, or liposome-mediated delivery could be used. Expression of the antibodies may be carried out in any suitable type of host cells, such as bacterial cells, yeast, insect cells, and mammalian cells. The antibodies of the invention may also be expressed using including in vitro transcription/translation systems. All of such methods are well known by those skilled in the art, and one of skill in the art would readily be able to select a suitable method depending on the nature of the nucleotide sequences, vectors, and cell types used.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the antibody or antigen binding fragment thereof may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

The skilled artisan will understand from the foregoing description that the present disclosure also provides an isolated nucleic acid encoding an antibody or antigen binding fragment thereof) of the present disclosure.

The present disclosure also provides an expression construct comprising an isolated nucleic acid of the disclosure operably linked to a promoter. In one example, the expression construct is an expression vector.

In one example, the expression construct of the disclosure comprises a nucleic acid encoding a polypeptide (e.g., comprising a V_{H}) operably linked to a promoter and a nucleic acid encoding another polypeptide (e.g., comprising a V_{L}) operably linked to a promoter.

The disclosure also provides a host cell comprising an expression construct according to the present disclosure.

The T present disclosure also provides an isolated cell expressing an antibody or antigen binding fragment thereof of the disclosure or a recombinant cell genetically-modified to express the antibody or antigen binding fragment thereof.

Methods for purifying antibodies or antigen binding fragments thereof according to the present disclosure are known in the art.

Where a peptide or polypeptide is secreted into the medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The the antibody or antigen binding fragment thereof prepared from cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).]

In one example, the antibody or antigen binding fragment thereof of the present disclosure is conjugated to another compound. The antibody or antigen binding fragment thereof can be directly or indirectly bound to the compound (e.g., can comprise a linker in the case of indirect binding). Examples of compounds include, a radioisotope (e.g., iodine-131, yttrium-90 or indium-111), a detectable label (e.g., a fluorophore or a fluorescent nanocrystal or quantum dot), a therapeutic compound (e.g., a chemotherapeutic or an anti-inflammatory), a colloid (e.g., gold), a toxin (e.g., ricin or tetanus toxoid), a nucleic acid, a peptide (e.g., a serum albumin binding peptide), a protein (e.g., a protein comprising an antigen binding domain of an antibody or serum albumin), an agent that increases the half-life of the compound in a subject (e.g., polyethylene glycol or other water soluble polymer having this activity) and mixtures thereof.

Methods for attaching a drug or other small molecule pharmaceutical to an antibody are well known and can include use of bifunctional chemical linkers such as N-succinimidyl (4-iodoacetyl)-aminobenzoate; sulfosuccinimidyl (4-iodoacetyl)-aminobenzoate; 4-succinimidyl-oxycarbonyl-(2-pyridyldithio) toluene; sulfosuccinimidyl-6-[.alpha.-methyl-. A-inverted.-(pyridyldithiol)-toluami-do]hexanoate; N-succinimidyl-3-(-2-pyridyldithio)-proprionate; succinimidyl-6-[3 (-(-2-pyridyldithio)-proprionamido]hexanoate; sulfosuccinimidyl-6-[3 (-(-2-pyridyldithio)-propionamido]hexanoate; 3-(2-pyridyldithio)-propionyl hydrazide, Ellman's reagent, dichlorotriazinic acid, S-(2-thiopyridyl)-L-cysteine, and the like. Further bifunctional linking molecules are discussed in, for example, U.S. Pat. Nos. 5,349,066, 5,618,528, 4,569,789, 4,952,394, and 5,137,877.

The linker can cleavable or noncleavable. Highly stable linkers can reduce the amount of payload that falls off in circulation, thus improving the safety profile, and ensuring that more of the payload arrives at the target cell. Linkers can be based on chemical motifs including disulfides, hydrazones or peptides (cleavable), or thioethers (noncleavable) and control the distribution and delivery of the active agent to the target cell. Cleavable and noncleavable types of linkers have been proven to be safe in preclinical and clinical trials (see, e.g., Brentuximab vedotin which includes an enzyme-sensitive linker cleavable by cathepsin; and Trastuzumab emtansine, which includes a stable, non-cleavable linker). In particular embodiments, the linker is a peptide linker cleavable by Edman degredation (B gchor, et al., Molecular diversity, 17 (3): 605-11 (2013)).

A non-cleavable linker can keep the active agent within the cell or the target microenvironment. As a result, the entire antibody, linker and active agent enter the targeted cell where the antibody is degraded to the level of an amino acid. The resulting complex between the amino acid of the antibody, the linker and the active agent becomes the active drug. In contrast, cleavable linkers are catalyzed by enzymes in the target cell or microenvironment where it releases the active agent. Once cleaved, the payload can escape from the targeted cell and attack neighboring cells (also referred to as “bystander killing”). In the case of the disclosed antibodies or antigen binding fragments thereof, cleavage of the linker can lead to two active agents, the antibody or antigen binding fragment thereof itself and its payload, which can have different mechanisms of action in the target cell or microenvironment.

In some embodiments, there is one or more additional molecules, between the active agent and the cleavage site. Other considerations include site-specific conjugation (TDCs) (Axup, Proceedings of the National Academy of Sciences, 109 (40): 16101-6 (2012) and conjugation techniques such as those described in Lyon, et al., Bioconjugate Chem., 32 (10): 1059-1062 (2014), and Kolodych, et al., Bioconjugate Chem., 26 (2): 197-200 (2015) which can improve stability and therapeutic index, and .alpha. emitting immunoconjugates (Wulbrand, et al., Multhoff, Gabriele, ed., PLOS ONE. 8 (5): c64730 (2013)).

In an example, the antibody or antigen binding fragment thereof is conjugated to nanoparticles or microparticles (for example as reviewed in Kogan et al., Nanomedicine (Lond). 2:287-306, 2007). The nanoparticles may be metallic nanoparticles. The particles can be polymeric particles, liposomes, micelles, microbubbles, and other carriers and delivery vehicles known in the art.

If the delivery vehicle is a polymeric particle, the antibody or antigen binding fragment thereof can be coupled directly to the particle or to an adaptor element such as a fatty acid which is incorporated into the polymer. Ligands may be attached to the surface of polymeric particles via a functional chemical group (carboxylic acids, aldehydes, amines, sulfhydryls and hydroxyls) present on the surface of the particle and present on the ligand to be attached. Functionality may be introduced post-particle preparation, by crosslinking of particles and ligands with homo- or heterobifunctional crosslinkers. This procedure may use a suitable chemistry and a class of crosslinkers (CDT, EDAC, glutaraldehydes, etc. as discussed in more detail below) or any other crosslinker that couples ligands to the particle surface via chemical modification of the particle surface after preparation.

The antibody or antigen binding fragment thereof may also be attached to polymeric particles indirectly though adaptor elements which interact with the polymeric particle. Adaptor elements may be attached to polymeric particles in at least two ways. The first is during the preparation of the micro- and nanoparticles, for example, by incorporation of stabilizers with functional chemical groups during emulsion preparation of microparticles. For example, adaptor elements, such as fatty acids, hydrophobic or amphiphilic peptides and polypeptides can be inserted into the particles during emulsion preparation. In a second embodiment, adaptor elements may be amphiphilic molecules such as fatty acids or lipids which may be passively adsorbed and adhered to the particle surface, thereby introducing functional end groups for tethering to the antibody or antigen binding fragment thereof. Adaptor elements may associate with micro- and nanoparticles through a variety of interactions including, but not limited to, hydrophobic interactions, electrostatic interactions and covalent coupling.

Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for particles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof.

In one example, an antibody or antigen binding fragment thereof of the disclosure is conjugated to a chemotherapy agent.

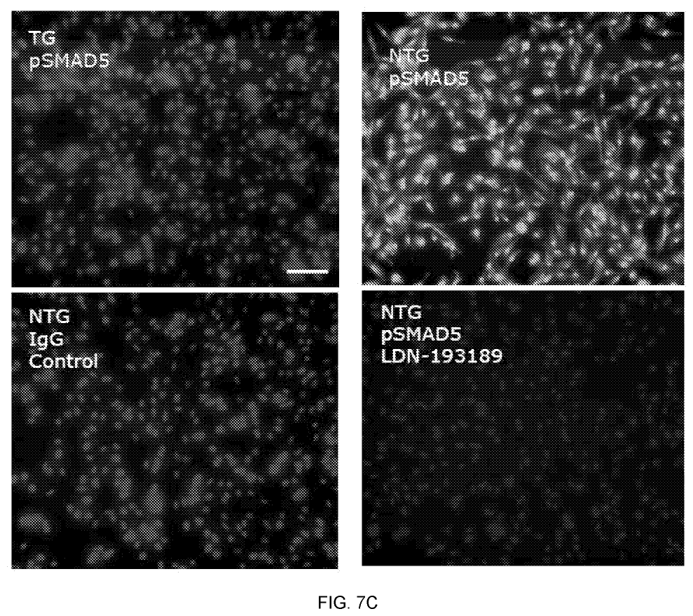

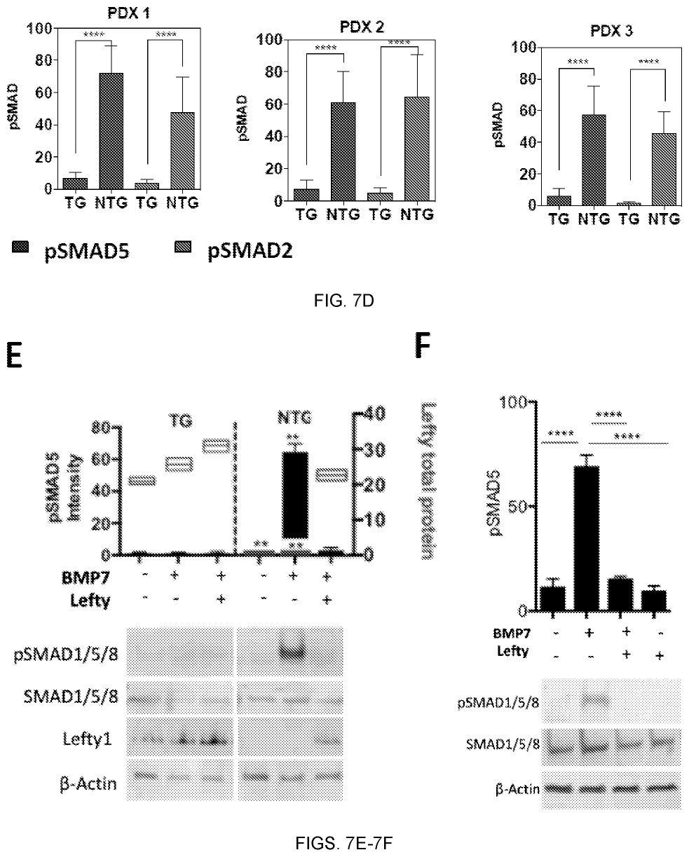

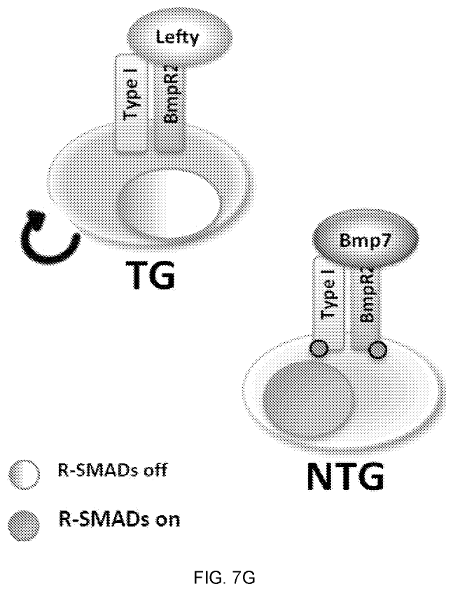

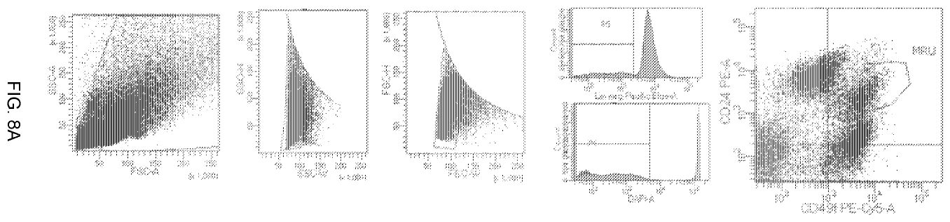

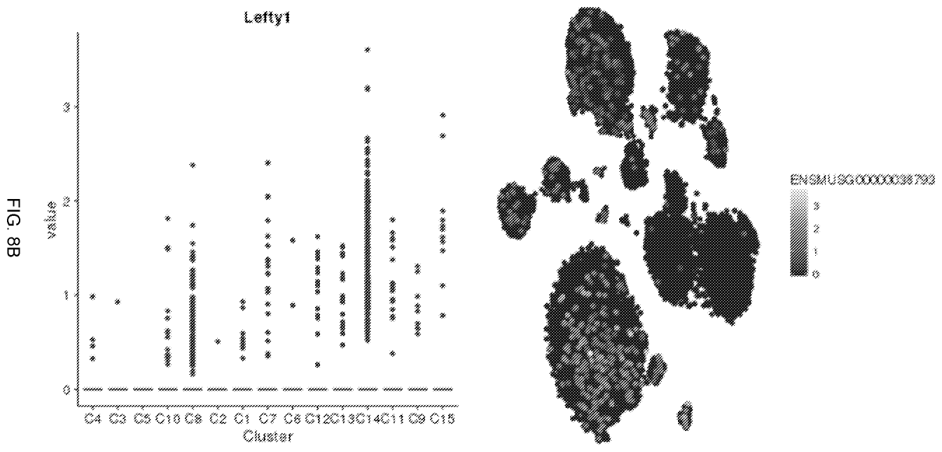

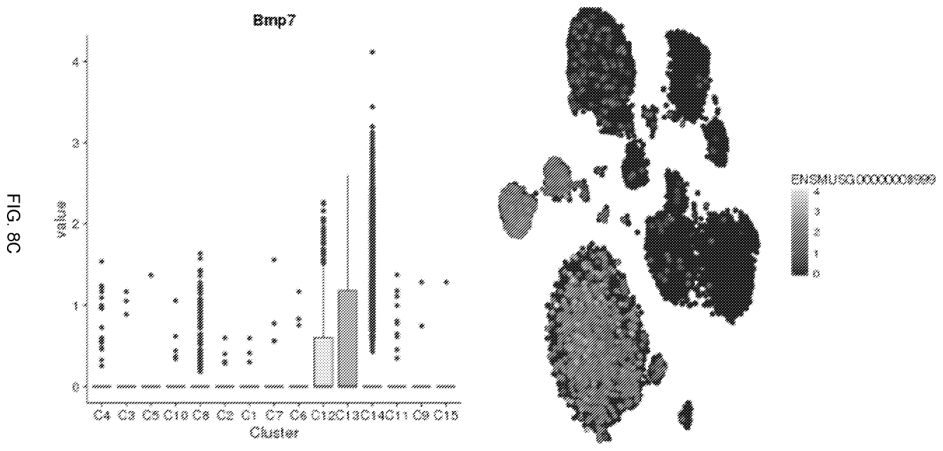

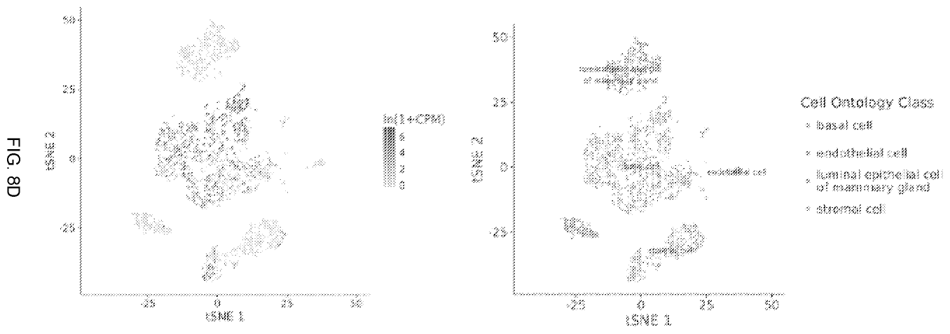

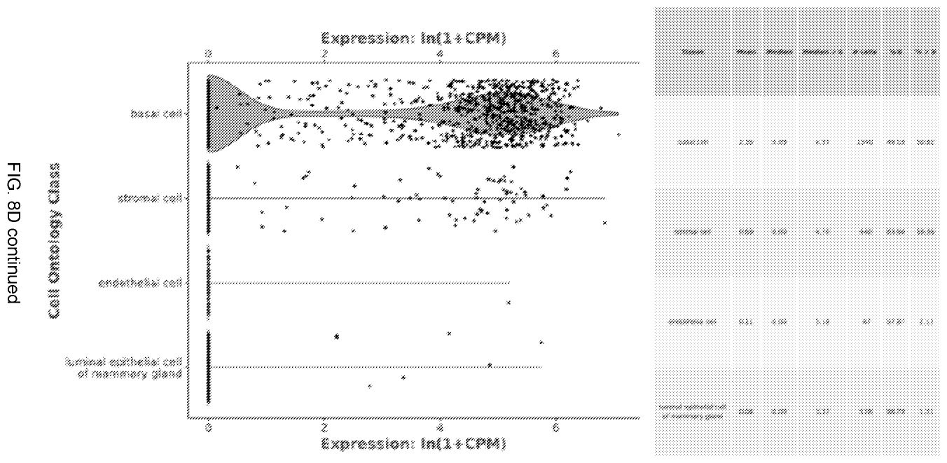

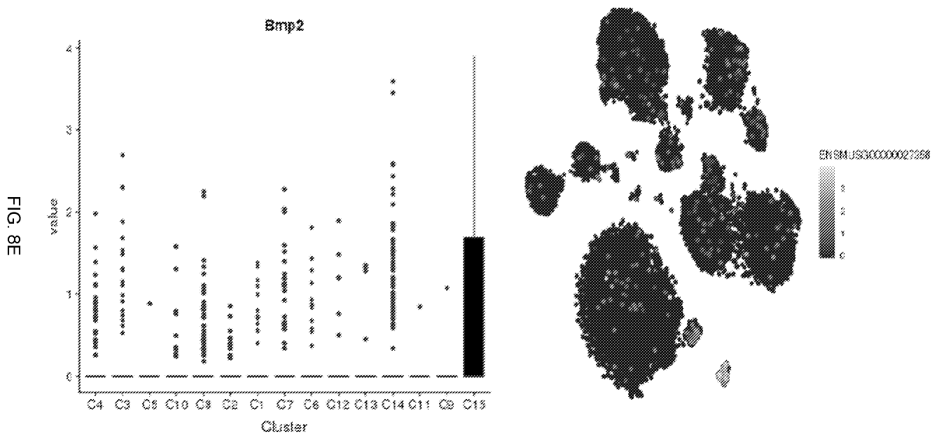

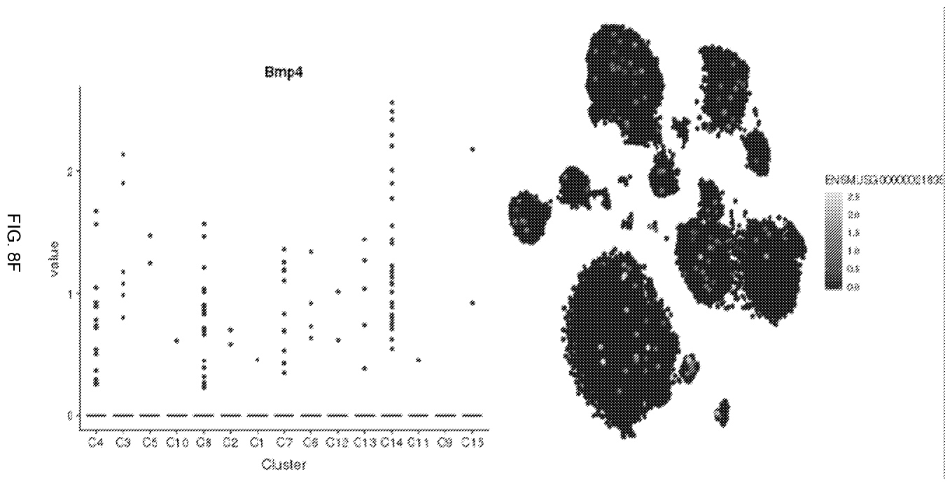

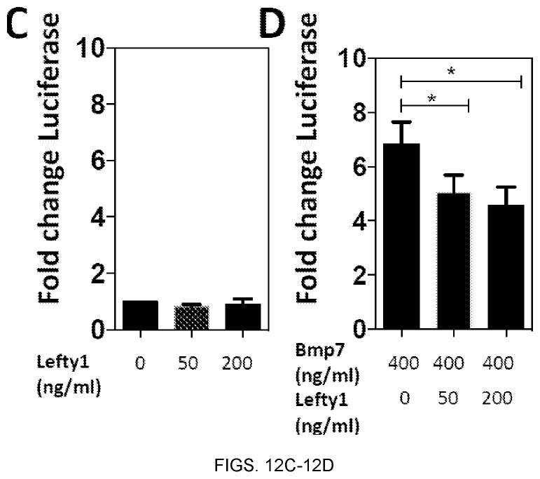

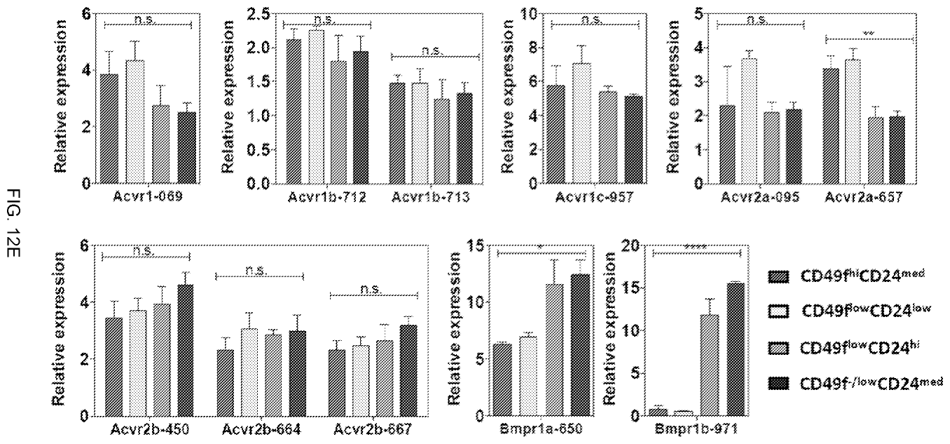

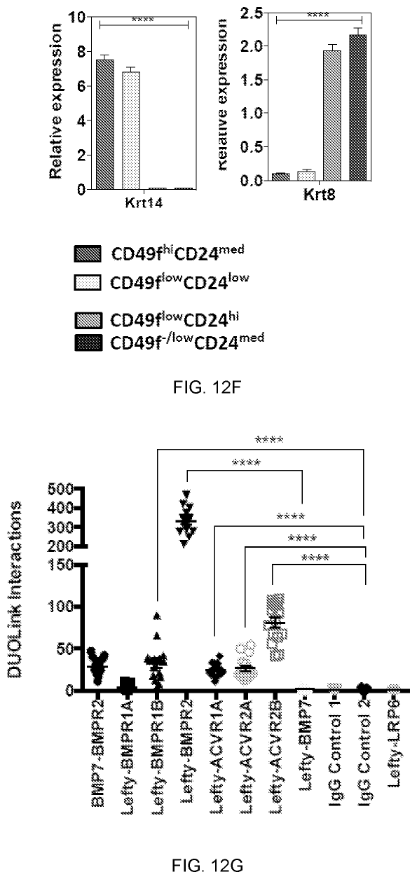

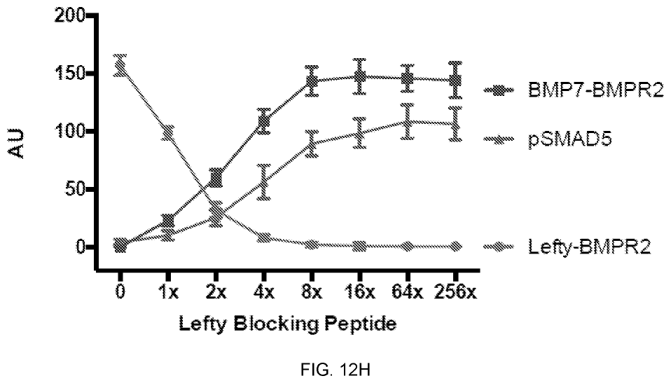

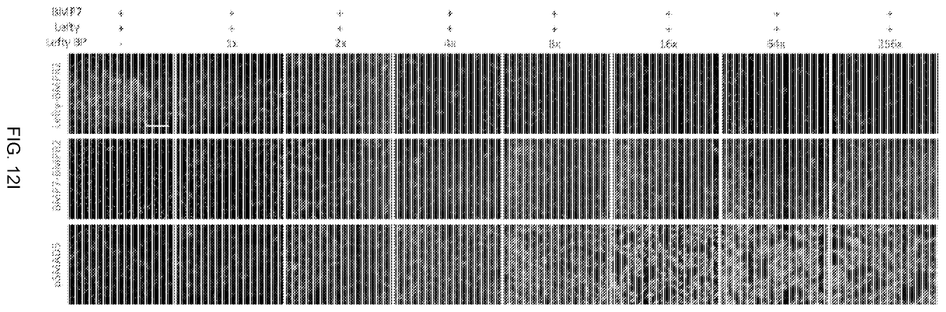

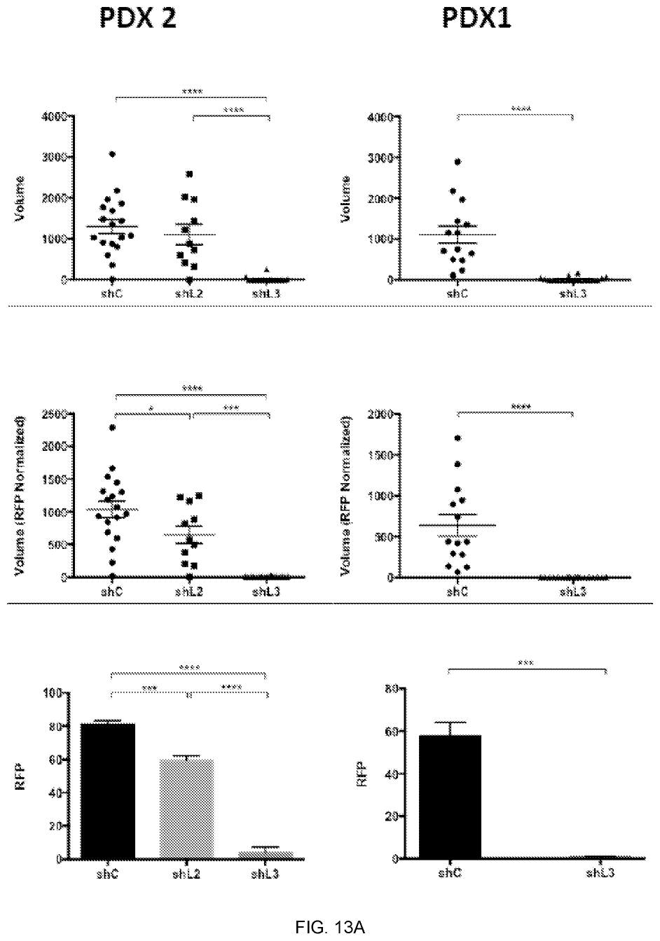

The present invention also encompasses the use of an antibody or antigen binding fragment thereof to increase the expression or activation of SMAD2 and/or SMAD5. LEFTY1 simultaneously suppresses SMAD2 and SMAD5 activation in drug resistant cells (FIG. 7). Therefore, neutralizing LEFTY proteins activates SMAD2 and SMAD5. An example of this is shown (FIG. 12) where breast cancer cells that are exposed to a LEFTY blocking peptide increases the activation of SMAD5 in a dose dependent fashion.

Suitably, in compositions or methods for administration of an antibody or antigen binding fragment thereof according to the present disclosure to a subject, the antibody or antigen binding fragment thereof is combined with a pharmaceutically acceptable carrier as is understood in the art. In one example, the present disclosure provides a composition (e.g., a pharmaceutical composition) comprising an antibody or antigen binding fragment thereof of the disclosure combined with a pharmaceutically acceptable carrier. In another example, the disclosure provides a kit comprising a pharmaceutically acceptable carrier suitable for combining or mixing with an antibody or antigen binding fragment thereof prior to administration to the subject. In this example, the kit may further comprise instructions for use.

The pharmaceutical compositions of the present invention preferably comprise a pharmaceutical effective amount of at least one antibody or antigen binding fragment thereof or functional equivalent thereof specifically recognizing an epitope within the C-terminus of LEFTY. A pharmaceutical effective amount is an amount of an antibody or antigen binding fragment thereof, which in induces the desired response in an individual receiving said pharmaceutical composition.

The pharmaceutically effective amount of the antibody or antigen binding fragment thereof depends on the individual to which it should be administered, in particular on the size of said individual as well as the clinical condition and the specific mode of administration. In general however, in the range of 1 mg to 5000 mg, preferably in the range of 10 mg to 3000 mg, more preferably in the range of 50 mg to 1000 mg, for example in the range of 100 mg to 750 mg, such as in the range of 150 mg to 500 mg. for example in the range of 200 mg to 400 mg, such as in the range of 250 mg to 350 mg. for example around 300 mg of the antibody or antigen binding fragment thereof should be administered to an adult human being per dosc.

In another embodiment, fixed dosing of monoclonal antibodies may be adopted (see, e.g., Hendrikx et al., Oncologist. 2017 October; 22 (10): 1212-1221). Most monoclonal antibodies in oncology are administered in body-size-based dosing schedules. This is believed to correct for variability in both drug distribution and elimination between patients. However, monoclonal antibodies typically distribute to the blood plasma and extracellular fluids only, which increase less than proportionally with the increase in body weight. Elimination takes place via proteolytic catabolisma nonspecific immunoglobulin G elimination pathway, and intracellular degradation after binding to the target. The latter is the primary route of elimination and is related to target expression levels rather than body size. Taken together, the minor effects of body size on distribution and elimination of monoclonal antibodies and their usually wide therapeutic window do not support body-size-based dosing. The below table provides monoclonal antibodies approved for treatment of cancer and a proposal for fixed dosing.

| | | | | | | | | Corresponding |
|---|---|---|---|---|---|---|---|---|
| | | | Volume of | Body weight | | | | body size |
| | | | distribution | effect on | | Body weight | | based dose |
| | | Therapeutic | at steady | volume of | Clearance | effect on | Proposed | after fixed |
| Generic name | Approved dose | window [symbol] | state (L) | distribution [symbol] | (L/day) | clearance [symbol] | fixed dose | dosing |
| Bevacinumab | 5 mg/kg; 2 weekly | 5-15 | 2. [symbol] | 0.411 | 0.207 | 0.368 | 40-140 kg; 600 | 4.2-15 |
| | 10 mg/kg; 2 weekly | mg/kg | | | | | mg, 2 weekly | mg/kg |
| | 15 mg/kg; 3 weekly | | | | | | | |
| Ca [symbol] | Day 0: 10 ug | Intra [symbol] administration with limited absorption | | | | | Approved | |
| | Day 3: 20 ug | into the systemic circulation. | | | | | fixed dose | |
| | Day 7: 50 ug | | | | | | | |
| | Day 10: 150 ug | | | | | | | |
| Ce [symbol] | 250 mg/m² weekly | 250-400 | 5.22 | 0.42 (effect | 0.497 | None | 1.3-2.2 m²; 500 | 22 [symbol] -3 [symbol] mg/m² |
| | (400 mg/m² | mg/m² | | of [symbol] was | | | mg, weekly (with | (364-615 mg/m² |
| | loading dose) | | | evaluated) | | | 800 mg loading | loading dose) |
| | | | | | | | dose) | |
| [symbol] | 3 mg/kg; [symbol] weekly | 3-10 | 4.15 | 0.708 | 0.360 | 0.642 | 40-60 kg; 150 | 2.5-3.8 mg/kg |
| | | mg/kg | | | | | mg, 3 weekly | 2.5-4.2 mg/kg |
| | | | | | | | 60-100 kg; 250 | 2.5-3.5 mg/kg |
| | | | | | | | mg, 3 weekly | |
| | | | | | | | 100-140 kg; 350 | |
| | | | | | | | mg, 3 weekly | |
| [symbol] | 3 mg/kg, 2 weekly | 1-10 | 8.0 | 0.580 | 0.128 | 0.707 | 40-140 kg; 240 | 1.7-6 |
| | | mg/kg | | | | | mg, 2 weekly | mg/kg |
| [symbol] | 1 [symbol] 000 mg per cycle | 1.0 [symbol] 0-2,000 | 2.75 | 0.383 | 0.083 | 0. [symbol] | Approved | |
| | (cycle 2-6) | mg | | | | | fixed dose | |
| Ofat [symbol] | 1,000 mg; 4 weekly | 1,000-2,000 | 3.26 | 0.0 [symbol] | 0.369 | 0.229 | Approved | |
| | [untreated C [symbol] ] | mg | | | | | fixed dose | |
| | 2,000 mg; weekly | | | | | | | |
| | [refractory C [symbol] ] | | | | | | | |
| Pan [symbol] | 6 mg/kg; 2 weekly | 2.5-9 | 3. [symbol] | 0.526 | 0.269 | 0.411 | 40-80 kg; 300 | 3.75-7.5 mg/kg |
| | | mg/kg | | | | | mg, 2 weekly | 3.5-6.25 mg/kg |
| | | | | | | | 80-140 kg; 500 | |
| | | | | | | | mg, 2 weekly | |
| Pe [symbol] | 2 mg/kg; 3 weekly | 1-10 | 8.1 | 0.489 | 0.23 | 0.595 | 40-140 kg; 150 | 1.1-3.8 |
| | | mg/kg | | | | | mg, 3 weekly | mg/kg |
| Per [symbol] | 420 mg; 3 weekly | 420- [symbol] | 3.07 | 0.747 | 0.239 | 0.5 [symbol] 6-0.5 [symbol] | Approved | |
| | (840 mg | mg | | | | | fixed dose | |
| | loading dose) | | | | | | | |
| Ra [symbol] | [symbol] mg/kg; 2 weekly | 8-10 | 5.5 | Not | 0.3 [symbol] | Not | Insufficient | |
| | | mg/kg | | reported | | reported | data | |
| Ri [symbol] | 375 mg/m², interval | 375-2,250 | 2.9 [symbol] | 0.73 | 0.257 | 1.02 | 1.3-2.2 m²; 800 | 364-635 |
| | [symbol] variable | mg | | | | | mg per administration | mg/m³ |
| [symbol] | 2 mg/kg/week | 1-2.8 | 2.9 [symbol] | 0. [symbol] 56 | 0.225 | 1.07 | 40-140 kg; 450 | 3.2-11.3 |
| | [with an | mg/kg | | | | | mg, 3 weekly | mg/kg |
| | additional | | | | | | | |
| | 2 mg/kg as | | | | | | | |
| | loading dose] | | | | | | | |

[symbol]

indicates data missing or illegible when filed

The dosage of LEFTY monoclonal antibodies may be extrapolated from the above data. Alternatively, the present invention also includes administering anti-cancer drugs, such as the monoclonal antibodies listed above.

The present invention contemplates administering a LEFTY antibody or antigen binding fragment thereof or a LEFTY antigen in combination with one or more cancer drugs. The cancer drug includes, but is not limited to, Abemaciclib, Abiraterone Acetate, Abraxane (Paclitaxel Albumin-stabilized Nanoparticle Formulation), ABVD, ABVE, ABVE-PC, AC, Acalabrutinib, AC-T, Actemra (Tocilizumab), Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alpelisib, Alunbrig (Brigatinib), Ameluz (Aminolevulinic Acid Hydrochloride), Amifostine, Aminolevulinic Acid Hydrochloride, Anastrozole, Apalutamide, Aprepitant, Aranesp (Darbepoetin Alfa), Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Asparlas (Calaspargase Pegol-mkn1), Atezolizumab, Avapritinib, Avastin (Bevacizumab), Avelumab, Axicabtagene Ciloleucel, Axitinib, Ayvakit (Avapritinib), Azacitidine, Azedra (Iobenguane I 131), Balversa (Erdafitinib), Bavencio (Avelumab), BEACOPP, Beleodaq (Belinostat), Belinostat, Bendamustine Hydrochloride. Bendeka (Bendamustine Hydrochloride), BEP, Besponsa (Inotuzumab Ozogamicin), Bevacizumab, Bexarotene, Bicalutamide, BiCNU (Carmustinc), Binimctinib, Bleomycin Sulfate, Blinatumomab, Blincyto (Blinatumomab), Bortezomib, Bosulif (Bosutinib), Bosutinib, Braftovi (Encorafenib), Brentuximab Vedotin, Brigatinib, Brukinsa (Zanubrutinib), BuMel, Busulfan, Busulfex (Busulfan), Cabazitaxel, Cablivi (Caplacizumab-yhdp), Cabometyx (Cabozantinib-S-Malate), Cabozantinib-S-Malate, CAF, Calaspargase Pegol-mkn1, Calquence (Acalabrutinib), Campath (Alemtuzumab), Camptosar (Irinotecan Hydrochloride), Capecitabine, Caplacizumab-yhdp. Capmatinib Hydrochloride, CAPOX, Carac (Fluorouracil—Topical), Carboplatin, CARBOPLATIN-TAXOL, Carfilzomib, Carmustine, Carmustine Implant, Casodex (Bicalutamide), CEM, Cemiplimab-rwlc. Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clofarabine, Clolar (Clofarabine), CMF. Cobimetinib Fumarate, Cometriq (Cabozantinib-S-Malate), Copanlisib Hydrochloride, COPDAC, Copiktra (Duvelisib), COPP, COPP-ABV. Cosmegen (Dactinomycin), Cotellic (Cobimetinib Fumarate), Crizotinib, CVP, Cyclophosphamide, Cyramza (Ramucirumab), Cytarabine, Dabrafenib Mesylate, Dacarbazine, Dacogen (Decitabine), Dacomitinib, Dactinomycin, Daratumumab, Daratumumab and Hyaluronidase-fihj. Darbepoetin Alfa, Darolutamide, Darzalex (Daratumumab), Darzalex Faspro (Daratumumab and Hyaluronidase-fihj), Dasatinib, Daunorubicin Hydrochloride, Daunorubicin Hydrochloride and Cytarabinc Liposome, Daurismo (Glasdegib Maleate), Decitabine, Defibrotide Sodium, Defitelio (Defibrotide Sodium), Degarelix, Denileukin Diftitox, Denosumab, Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Durvalumab, Duvelisib, Efudex (Fluorouracil—Topical), Eligard (Leuprolide Acetate), Elitek (Rasburicase), Ellence (Epirubicin Hydrochloride), Elotuzumab, Eloxatin (Oxaliplatin), Eltrombopag Olamine, Elzonris (Tagraxofusp-erzs), Emapalumab-Izsg. Emend (Aprepitant), Empliciti (Elotuzumab), Enasidenib Mesylate, Encorafenib, Enfortumab Vedotin-ejfv, Enhertu (Fam-Trastuzumab Deruxtecan-nxki), Entrectinib, Enzalutamide, Epirubicin Hydrochloride, EPOCH, Epoctin Alfa, Epogen (Epoctin Alfa), Erbitux (Cetuximab), Erdafitinib, Eribulin Mesylate, Erivedge (Vismodegib), Erleada (Apalutamide), Erlotinib Hydrochloride, Erwinaze (Asparaginase Erwinia chrysanthemi), Ethyol (Amifostine), Etopophos (Etoposide Phosphate), Etoposide, Etoposide Phosphate, Everolimus, Evista (Raloxifene Hydrochloride), Evomela (Melphalan Hydrochloride), Exemestane, 5-FU (Fluorouracil Injection), 5-FU (Fluorouracil—Topical), Fam-Trastuzumab Deruxtecan-nxki, Fareston (Toremifene), Farydak (Panobinostat), Faslodex (Fulvestrant), FEC, Fedratinib Hydrochloride, Femara (Letrozole), Filgrastim, Firmagon (Degarelix), Fludarabine Phosphate, Fluoroplex (Fluorouracil—Topical), Fluorouracil Injection, Fluorouracil—Topical, Flutamide, FOLFIRI, FOLFIRI-BEVACIZUMAB, FOLFIRI-CETUXIMAB, FOLFIRINOX, FOLFOX, Folotyn (Pralatrexate), Fostamatinib Disodium, FU-LV, Fulvestrant, Gamifant (Emapalumab-lzsg), Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gilteritinib Fumarate, Glasdegib Maleate, Gleevec (Imatinib Mesylate), Gliadel Wafer (Carmustine Implant), Glucarpidase, Goserelin Acetate, Granisetron, Granisetron Hydrochloride, Granix (Filgrastim), Halaven (Eribulin Mesylate), Hemangeol (Propranolol Hydrochloride), Herceptin Hylecta (Trastuzumab and Hyaluronidase-oysk), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurca), Hydroxyurca, Hyper-CVAD. Ibrance (Palbociclib), Ibritumomab Tiuxetan, Ibrutinib, ICE, Iclusig (Ponatinib Hydrochloride), Idamycin PFS (Idarubicin Hydrochloride), Idarubicin Hydrochloride, Idelalisib, Idhifa (Enasidenib Mesylate), Ifex (Ifosfamide), Ifosfamide, IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Inrebic (Fedratinib Hydrochloride), Interferon Alfa-2b, Recombinant, Interleukin-2 (Aldesleukin), Intron A (Recombinant Interferon Alfa-2b), Iobenguane I 131, Ipilimumab, Iressa (Gefitinib), Irinotecan Hydrochloride, Irinotecan Hydrochloride Liposome, Isatuximab-irfc, Istodax (Romidepsin), Ivosidenib, Ixabepilone. Ixazomib Citrate, Ixempra (Ixabepilone), Jakafi (Ruxolitinib Phosphate), JEB. Jelmyto (Mitomycin), Jevtana (Cabazitaxel), Kadcyla (Ado-Trastuzumab Emtansine), Kepivance (Palifermin), Keytruda (Pembrolizumab), Kisqali (Ribociclib), Koselugo (Selumetinib Sulfate), Kymriah (Tisagenlecleucel), Kyprolis (Carfilzomib), Lanreotide Acetate, Lapatinib Ditosylate, Larotrectinib Sulfate, Lenalidomide, Lenvatinib Mesylate, Lenvima (Lenvatinib Mesylate), Letrozole, Leucovorin Calcium, Leukeran (Chlorambucil), Leuprolide Acetate, Levulan Kerastik (Aminolevulinic Acid Hydrochloride), Libtayo (Cemiplimab-rwlc), Lomustine, Lonsurf (Trifluridine and Tipiracil Hydrochloride), Lorbrena (Lorlatinib), Lorlatinib, Lumoxiti (Moxctumomab Pasudotox-tdfk), Lupron (Leuprolide Acetate), Lupron Depot (Leuprolide Acetate), Lurbinectedin, Luspatercept-aamt, Lutathera (Lutetium Lu 177-Dotatate), Lutetium (Lu 177-Dotatate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Mektovi (Binimetinib), Melphalan, Melphalan Hydrochloride, Mercaptopurine, Mesna, Mesnex (Mesna), Methotrexate, Methylnaltrexone Bromide, Midostaurin, Mitomycin, Mitoxantrone Hydrochloride, Mogamulizumab-kpkc. Moxctumomab Pasudotox-tdfk, Mozobil (Plerixafor), Mustargen (Mechlorethamine Hydrochloride), MVAC. Mvasi (Bevacizumab), Myleran (Busulfan), Mylotarg (Gemtuzumab Ozogamicin), Nanoparticle Paclitaxel (Paclitaxel Albumin-stabilized Nanoparticle Formulation), Necitumumab, Nelarabine, Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nplate (Romiplostim), Nubeqa (Darolutamide), Obinutuzumab, Odomzo (Sonidegib), OEPA, Ofatumumab, OFF. Olaparib, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib Mesylate, Oxaliplatin, Paclitaxel, Paclitaxel Albumin-stabilized Nanoparticle Formulation, PAD, Padcev (Enfortumab Vedotin-cjfv), Palbociclib, Palifermin, Palonosetron Hydrochloride, Palonosetron Hydrochloride and Netupitant, Pamidronate Disodium, Panitumumab, Panobinostat, Pazopanib Hydrochloride, PCV, PEB, Pegaspargase, Pegfilgrastim, Peginterferon Alfa-2b, PEG-Intron (Peginterferon Alfa-2b), Pemazyre (Pemigatinib), Pembrolizumab, Pemetrexed Disodium, Pemigatinib, Perjeta (Pertuzumab), Pertuzumab, Pertuzumab, Trastuzumab, and Hyaluronidase-zzxf, Pexidartinib Hydrochloride, Phesgo (Pertuzumab, Trastuzumab, and Hyaluronidase-zzxf), Piqray (Alpelisib), Plerixafor, Polatuzumab Vedotin-piiq, Polivy (Polatuzumab Vedotin-piiq), Pomalidomide, Pomalyst (Pomalidomide), Ponatinib Hydrochloride, Portrazza (Necitumumab), Poteligeo (Mogamulizumab-kpkc), Pralatrexate, Prednisone, Procarbazine Hydrochloride, Procrit (Epoetin Alfa), Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Qinlock (Ripretinib), Radium 223 Dichloride, Raloxifene Hydrochloride, Ramucirumab, Rasburicase, Ravulizumab-cwvz, Reblozyl (Luspatercept-aamt), R-CHOP, R-CVP. Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Retacrit (Epoctin Alfa), Retevmo (Selpercatinib), Revlimid (Lenalidomide), Rheumatrex (Methotrexate), Ribociclib, R-ICE, Ripretinib, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rozlytrek (Entrectinib), Rubidomycin (Daunorubicin Hydrochloride), Rubraca (Rucaparib Camsylate), Rucaparib Camsylate, Ruxolitinib Phosphate, Rydapt (Midostaurin), Sacituzumab Govitecan-hziy, Sancuso (Granisetron), Sarclisa (Isatuximab-irfc), Sclerosol Intrapleural Aerosol (Talc), Selinexor, Selpercatinib, Selumctinib Sulfate, Siltuximab, Sipuleucel-T. Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sustol (Granisetron), Sutent (Sunitinib Malate), Sylatron (Peginterferon Alfa-2b), Sylvant (Siltuximab), Synribo (Omacetaxine Mepesuccinate), Tabloid (Thioguanine), Tabrecta (Capmatinib Hydrochloride), TAC. Tafinlar (Dabrafenib Mesylate), Tagraxofusp-erzs, Tagrisso (Osimertinib Mesylate), Talazoparib Tosylate, Talc, Talimogene Laherparepvec, Talzenna (Talazoparib Tosylate), Tamoxifen Citrate, Tarceva (Erlotinib Hydrochloride), Targretin (Bexarotene), Tasigna (Nilotinib), Tavalisse (Fostamatinib Disodium), Taxotere (Docetaxel), Tazemetostat Hydrobromide, Tazverik (Tazemetostat Hydrobromide), Tecentriq (Atezolizumab), Temodar (Temozolomide), Temozolomide, Temsirolimus, Thalidomide, Thalomid (Thalidomide), Thioguanine, Thiotepa, Tibsovo (Ivosidenib), Tisagenlecleucel, Tocilizumab, Tolak (Fluorouracil—Topical), Topotecan Hydrochloride, Toremifene, Torisel (Temsirolimus), Totect (Dexrazoxane Hydrochloride), TPF, Trabectedin, Trametinib, Trastuzumab, Trastuzumab and Hyaluronidase-oysk. Treanda (Bendamustine Hydrochloride), Trexall (Methotrexate), Trifluridine and Tipiracil Hydrochloride, Trisenox (Arsenic Trioxide), Trodelvy (Sacituzumab Govitecan-hziy), Truxima (Rituximab), Tucatinib, Tukysa (Tucatinib), Turalio (Pexidartinib Hydrochloride), Tykerb (Lapatinib Ditosylate), Ultomiris (Ravulizumab-cwvz), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velcade (Bortezomib), Vemurafenib, Venclexta (Venetoclax), Venetoclax, Verzenio (Abemaciclib), Vidaza (Azacitidine), Vinblastine Sulfate, Vincristine Sulfate, Vincristine Sulfate Liposome, Vinorelbine Tartrate, VIP, Vismodegib, Vistogard (Uridine Triacetate), Vitrakvi (Larotrectinib Sulfate), Vizimpro (Dacomitinib), Voraxaze (Glucarpidase), Vorinostat, Votrient (Pazopanib Hydrochloride), Vyxcos (Daunorubicin Hydrochloride and Cytarabine Liposome), Xalkori (Crizotinib), Xeloda (Capecitabine), XELIRI, XELOX, Xgeva (Denosumab), Xofigo (Radium 223 Dichloride), Xospata (Gilteritinib Fumarate), Xpovio (Selinexor), Xtandi (Enzalutamide), Yervoy (Ipilimumab), Yescarta (Axicabtagene Ciloleucel), Yondelis (Trabectedin), Zaltrap (Ziv-Aflibercept), Zanubrutinib, Zarxio (Filgrastim), Zejula (Niraparib Tosylate Monohydrate), Zelboraf (Vemurafenib), Zepzelca (Lurbinectedin), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and Zytiga (Abiraterone Acetate)

In a particularly advantageous embodiment, the cancer drug is a breast cancer drug. A breast cancer drug to prevent breast cancer includes, but is not limited to, Evista (Raloxifene Hydrochloride), Raloxifene Hydrochloride and Tamoxifen Citrate. A breast cancer drug to treat breast cancer includes, but is not limited to, Abemaciclib, Abraxane (Paclitaxel Albumin-stabilized Nanoparticle Formulation), Ado-Trastuzumab Emtansine, Afinitor (Everolimus), Afinitor Disperz (Everolimus), Alpelisib, Anastrozole, Aredia (Pamidronate Disodium), Arimidex (Anastrozole), Aromasin (Exemestane), Atezolizumab, Capecitabine, Cyclophosphamide, Docetaxel, Doxorubicin Hydrochloride, Ellence (Epirubicin Hydrochloride), Enhertu (Fam-Trastuzumab Deruxtecan-nxki), Epirubicin Hydrochloride, Eribulin Mesylate, Everolimus, Exemestane, 5-FU (Fluorouracil Injection), Fam-Trastuzumab Deruxtecan-nxki, Fareston (Toremifene), Faslodex (Fulvestrant), Femara (Letrozole), Fluorouracil Injection, Fulvestrant, Gemcitabine Hydrochloride, Gemzar (Gemcitabine Hydrochloride), Goserelin Acetate, Halaven (Eribulin Mesylate), Herceptin Hylecta (Trastuzumab and Hyaluronidase-oysk), Herceptin (Trastuzumab), Ibrance (Palbociclib), Ixabepilone, Ixempra (Ixabepilone), Kadcyla (Ado-Trastuzumab Emtansine), Kisqali (Ribociclib), Lapatinib Ditosylate, Letrozole, Lynparza (Olaparib), Megestrol Acetate, Methotrexate, Neratinib Maleate, Nerlynx (Neratinib Maleate), Olaparib, Paclitaxel, Paclitaxel Albumin-stabilized Nanoparticle Formulation, Palbociclib, Pamidronate Disodium, Perjeta (Pertuzumab), Pertuzumab, Pertuzumab, Trastuzumab, and Hyaluronidase-zzxf, Phesgo (Pertuzumab, Trastuzumab, and Hyaluronidase-zzxf), Piqray (Alpelisib), Ribociclib, Sacituzumab Govitecan-hziy, Talazoparib Tosylate, Talzenna (Talazoparib Tosylate), Tamoxifen Citrate, Taxotere (Docetaxel), Tecentriq (Atezolizumab), Thiotepa, Toremifene, Trastuzumab, Trastuzumab and Hyaluronidase-oysk, Trexall (Methotrexate), Trodelvy (Sacituzumab Govitecan-hziy), Tucatinib, Tukysa (Tucatinib), Tykerb (Lapatinib Ditosylate), Verzenio (Abemaciclib), Vinblastine Sulfate, Xeloda (Capecitabine) and Zoladex (Goserelin Acetate).

The composition of the present invention may be a pharmaceutical composition suitable for parenteral administration. Such compositions preferably, include aqueous and non-aqueous sterile injection solutions which may contain wetting or emulsifying reagents, anti-oxidants, pH buffering agents, bacteriostatic compounds and solutes which render the formulation isotonic with the body fluid, preferably the blood, of the individual; and aqueous and non-aqueous sterile suspensions which may include suspending agents or thickening agents. The pharmaceutical composition may be presented in unit-dose or multi-dose containers, for example, sealed ampoules and vials and may be stored in a freeze-dried condition requiring only the addition of the sterile liquid carrier immediately prior to use.

Preferably, the composition of the present invention comprises one or more suitable pharmaceutical excipients, which could be non-sterile or sterile, for use with cells, tissues or organisms, such as a pharmaceutical excipients suitable for administration to an individual. Such excipients may include, but are not limited to, saline, buffered saline, dextrose, water, glycerol, ethanol and combinations of these excipients in various amounts. The formulation should suit the mode of administration. The invention further relates to pharmaceutical kit of parts comprising one or more containers filled with one or more of the ingredients of the aforementioned compositions of the invention. Examples of non-aqueous excipients are propylene glycol, polyethylene glycol, vegetable oils such as olive oil, and injectable organic esters such as ethyl oleate.

Preferably, the pharmaceutical compositions of the present invention are prepared in a form which is injectable, either as liquid solutions or suspensions; furthermore solid forms suitable for solution in or suspension in liquid prior to injection are also within the scope of the present invention. The preparation may be emulsified or the immunogenic determinant as well as the collectins and/or collectin homologues according to the present invention may be encapsulated in liposomes.

The antibody or antigen binding fragment thereof may be administered alone or in combination with other compounds, either simultaneously or sequentially in any order

Administration could for example be parenteral injection or infusion, rapid infusion, nasopharyngeal absorption, dermal absorption, and enterally, such as oral administration.

Parenteral injection could for example be intravenous, intramuscular, intradermal or subcutaneous injection. Preferably, said administration is parenterally by injection or infusion.

The antibody or antigen binding fragment thereof should be administered as often as required, hence the antibody or antigen binding fragment thereof may be administered more than once, such as at least two times, for example at least 3 times, such as at least 4 times, for example at least 5 times, such as in the range of 1 to 100 times, for example in the range of 1 to 50 times, such as in the range of 1 to 25 times, for example in the range of 1 to 10 times.

Preferably, there is at least 1 day between 2 administrations, such as at least 2 days, for example at least 3 days, such as at least 5 days, for example at least one week, such as at least 2 weeks, for example at least one month, such as at least 6 months, for example at least 1 year, such at least 2 years, for example at least 3 years, such as at least 5 years, for example at least 10 years.

In some embodiments, the antibody or antigen binding fragment thereof is encapsulated or incorporated in nanoparticle, microparticle, or other delivery vehicle.

In some embodiments, antibody or antigen binding fragment thereof is utilized for detecting site or sites of cancer, tissue damage, injury, infection, or ischemia. The method typically including administering to a subject in need thereof an effective amount an agent that is detectable using diagnostic imaging or nuclear medicine techniques, and detecting the agent. In such methods, the agent is typically conjugated to the antibody or antigen binding fragment thereof or encapsulated in a delivery vehicle conjugated with the antibody or antigen binding fragment thereof. The diagnostic imaging or nuclear medicine technique can be, for example, PET-CT, bone scan, MRI, CT, echocardiography, ultrasound, and x-ray.

In an example, the antibody or antigen binding fragment thereof and compositions comprising the same can be used in the manufacture of a medicament for the treatment of a condition. In another example, the present disclosure relates to an antibody or antigen binding fragment thereof or compositions comprising the same for use in the treatment of a condition. Examples of conditions to be treated are discussed below.

The methods and uses typically include administering a subject in need there of an effective amount of an antibody or antigen binding fragment thereof. In some embodiments, the subject has cancer or virally infected or transformed cells. The methods and uses can include a combination therapy with a second, third, or more additional active agents. For example, the disclosed antibodies or antigen binding fragments thereof can be used in combination with standard chemotherapy, radiation therapy, and other anti-cancer treatments. Radiation therapy (a.k.a. radiotherapy) is the medical use of ionizing radiation as part of cancer treatment to control malignant cells.

Therapeutic combinations according to the present disclosure can be administered via various routes. Exemplary routes of administration include intravenous administration as a bolus or by continuous infusion over a period of time, intramuscular, intraperitoneal, intracerobrospinal, intrathecal, oral routes.

In an example, the antibody or antigen binding fragment thereof according to the present disclosure can be administered to a subject to treat various conditions.

In some examples of the disclosure, a method described herein is for the treatment of a cancer. The term “cancer” refers to or describes the physiological condition in mammals that is typically characterized by unregulated cell growth/proliferation. Examples of cancer include but are not limited to, carcinoma, lymphoma, blastoma, sarcoma, and leukemia or lymphoid malignancies. More particular examples of such cancers include, but are not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases. In another example, the term “cancer” encompasses triple negative breast cancer. Accordingly, in an example, the present disclosure relates to a method of treating breast, ovarian, colon, prostate, lung, brain, skin, liver, stomach, pancreatic or blood based cancer. In another example, the present disclosure relates to a method of treating Breast invasive carcinoma, liver carcinoma, cholangiocarcinoma, uterine carcinoma, ovarian carcinoma, melanoma, thymoma, lung adenocarcinoma, pheochromocytoma/paraganglioma, esophageal carcinoma, pancreatic carcinoma, glioblastoma multiforme, colorectal carcinoma, renal cell carcinoma or adrenal carcinoma.

In other examples, a method described herein is used to treat cancers that are linked to mutations in BRCA1, BRCA2, PALB2, OR RAD51B, RAD51C, RAD51D or related genes.

In a particularly advantageous embodiment, the antibodies and antigen binding fragments thereof is utilized in the detection, treatment and prevention of cancers wherein LEFTY1 is copy number amplified. Examples of such cancers include, but are not limited to, OV, Ovarian serous cystadenocarcinoma; BRCA.; Breast invasive carcinoma ESCA, Esophageal carcinoma; UCS, Uterine Carcinosarcoma; SKCM, Skin Cutaneous Melanoma; LIHC, Liver hepatocellular carcinoma; LUAD, Lung adenocarcinoma; BLCA, Bladder Urothelial Carcinoma; STAD, Stomach adenocarcinoma; LUSC, Lung squamous cell carcinoma; CHOL, Cholangiocarcinoma; UCEC, Uterine Corpus Endometrial Carcinoma; CESC, Cervical squamous cell carcinoma and endocervical adenocarcinoma; READ, Rectum adenocarcinoma; UVM, Uveal Melanoma, and MESO, Mesothelioma.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1: Interaction of LEFTY1 and BMPR2 Promotes Long-Term Proliferation of Normal and Malignant Mammary Cells

The studies presented here demonstrate that LEFTY1 stimulates and BMP7 antagonizes the growth of long-term mammary epithelial cells. Mammary progenitor cells produce LEFTY1, which acts as a natural inhibitor of both SMAD2 and SMAD5 signaling in vivo. LEFTY1 binds to BMPR2, attenuating BMP7 activation of SMAD5. Applicants find that a large percentage of human breast cancers overexpress LEFTY1. In breast cancer patient derived xenograft (PDX) models that contain a hierarchical proliferative paradigm, LEFTY1 is required for the growth of tumorigenic cells. The tumorigenic population from all of the PDXs that Applicants studied were decorated by cell-surface LEFTY1-BMPR2 interactions, whereas their non-tumorigenic counterparts were demarcated by BMP7-BMPR2 interactions. Applicants' data reveals that LEFTY1 is a novel endogenous dual-SMAD inhibiting protein that drives the proliferation of long-term tumorigenic breast cancer cells. Furthermore, Applicants propose that the LEFTY1/BMP7/BMPR2 signaling axis is a new pathway that regulates the self-renewal of normal and malignant breast epithelial cells.

Regulation of SMAD pathways has been linked to long-term cellular proliferation of epithelium. Here, Applicants discover that LEFTY1 is a ligand that concomitantly suppresses SMAD2 and SMAD5 signaling to regulate the long-term proliferative potential of normal and malignant mammary epithelial cells. BMP7 signaling by basal cells reduces the proliferation of progenitor cells in vitro and in vivo. In normal mouse breast epithelium, LEFTY1 expression by a subset of luminal cells and rare basal cells opposes BMP7 secreted by basal cells to promote ductal branching. Mechanistically, LEFTY1 binds to BMPR2 to suppress BMP7 induced activation of SMAD5. The LEFTY1-BMPR2 interaction is specific to tumor initiating cells in triple negative breast cancer xenografts that rely on LEFTY1 signaling for growth. Since the SMAD pathway has proven difficult to develop therapies against, Applicants' results suggest that inhibiting LEFTY1 function may present opportunities for targeted therapies for the treatment of breast cancer.

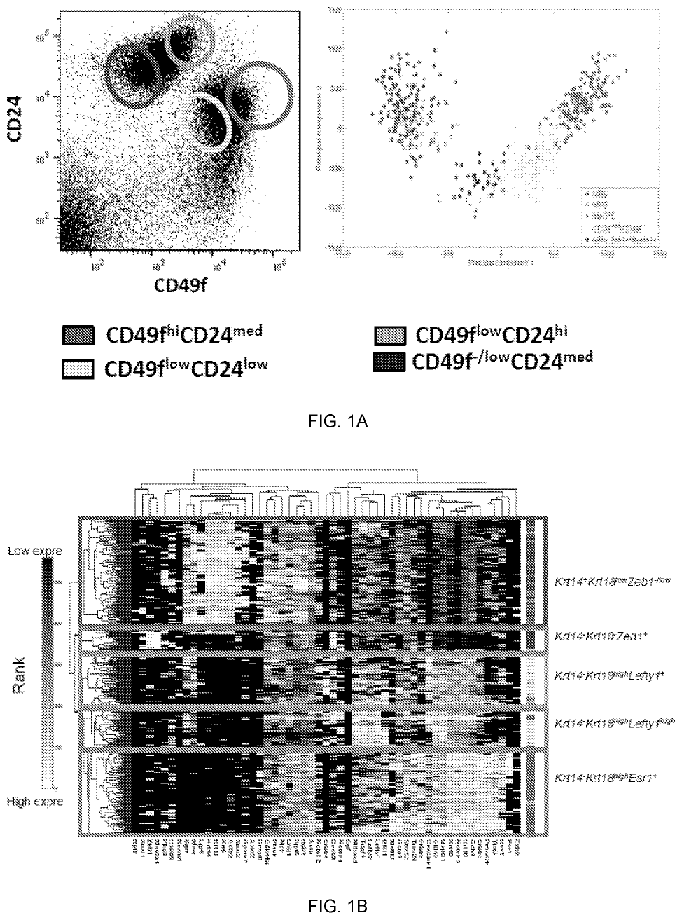

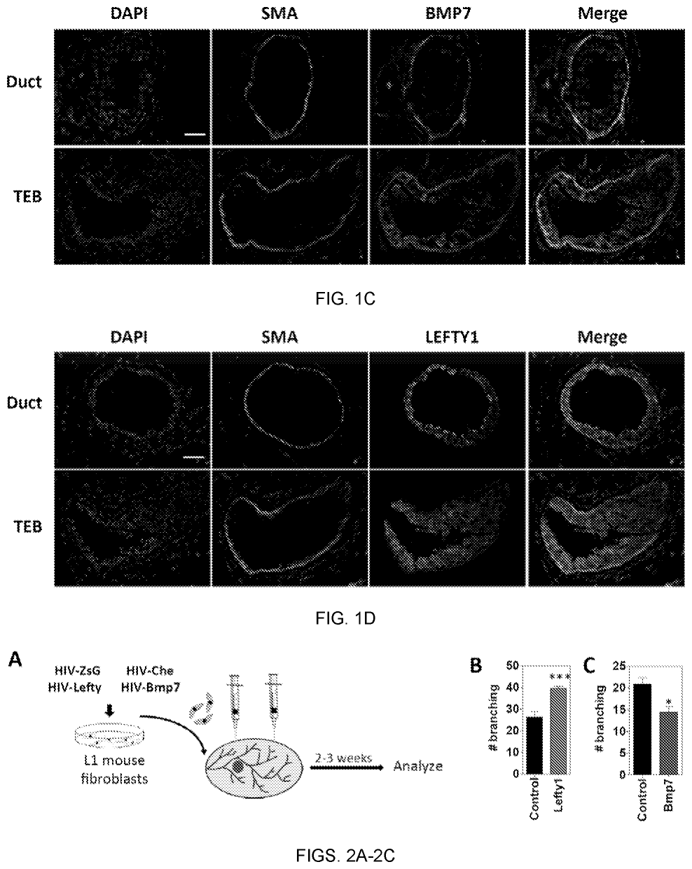

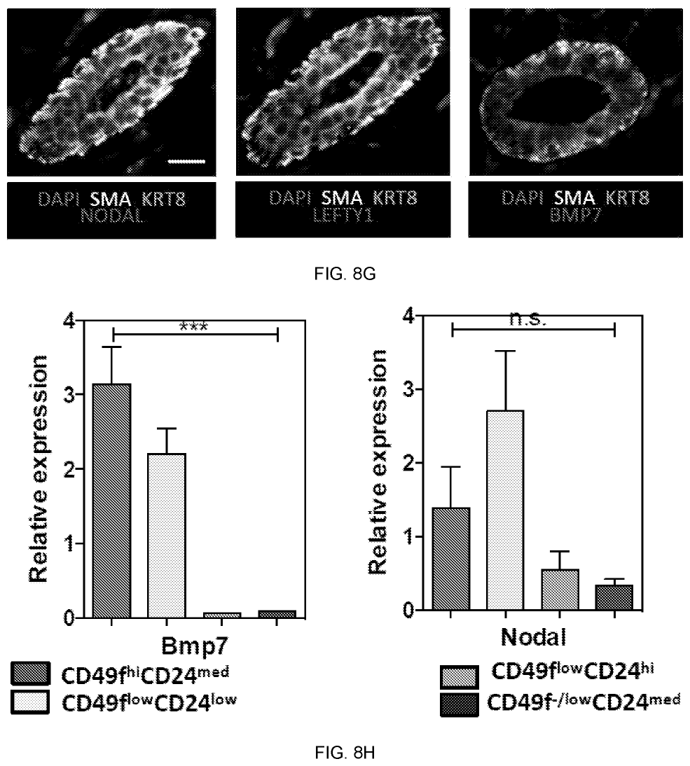

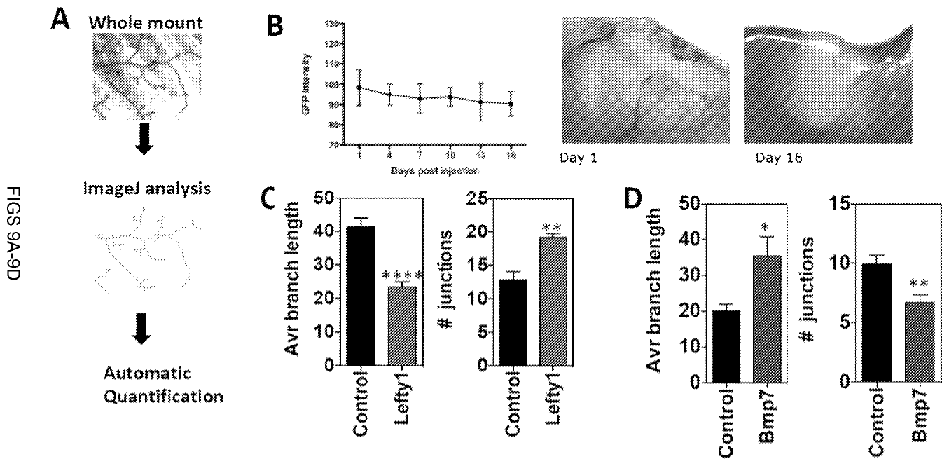

LEFTY1 promotes ductal branching and is opposed by secreted BMP7. A well-established f^{low} cytometric-sorting paradigm was utilized to isolate functionally distinct mammary cell types (FIGS. 1A and 8A) (Shackleton et al., 2006; Stingl et al., 2006). Single cell PCR was performed on sorted populations using a panel of genes whose expression profiles have been linked to stem cell self-renewal, differentiation and signaling unique to stem cells (FIG. 1B). Examination of single cell PCR gene expression data showed that Lefty1 is expressed by a subset of mammary gland epithelial cells (FIG. 1B). Lefty1's expression profile correlated most closely to those of Cripto-1 (Tdgf1) and Bmi-1, which are both genes that regulate the proliferation of long-term mammary cells (Pietersen et al., 2008; Spike et al., 2014) (FIG. 1B). The mRNA expression of Lefty1 in the mammary epithelial populations was further confirmed by the analysis of single cell RNA-seq data from the Marioni and Khaled groups (Bach et al., 2017) (FIG. 8B). Applicants found that the majority of highly expressing Lefty1 cells shared luminal markers. However, a minority population of basal cells that are enriched for an immature phenotype also expressed Lefty1. Strikingly, the expression of Bmp7 was restricted to the basal cells (FIGS. 8C-D) in the analysis by Bach et al., as well as the Tabula Muris single cell RNA-seq datasets (Schaum et al., 2018). The expression of other BMP family members such as Bmp2 and Bmp4 did not show much expression or compartmentalization (FIGS. 8E-F).

Applicants next sought to investigate whether exposure of the endogenous mammary epithelium to exogenous LEFTY1 or BMP7 could modulate mammary gland biology. Since NODAL and CRIPTO-1 have both been shown to promote mammary cell proliferation in vitro and in vivo (Klauzinska et al., 2015; Quail et al., 2012; Wechselberger et al., 2001), Applicants stained the mammary epithelium for these factors along with LEFTY1 and BMP7, using cytokeratin and SMAx expression to delineate the cellular compartments of the mammary gland (Deugnier et al., 2006). In situ, Applicants found that both KRT8⁺luminal cells and SMAα⁺basal cells expressed NODAL (FIG. 8G). In contrast, Applicants found a clear compartmentalization of BMP7 and LEFTY1 in some structures of the mammary epithelium. BMP7 was expressed in the SMAα⁺basal cells of ducts, and in both the basal and body cells of the ends of ducts (FIGS. 1C and 8G) whereas a subset of inner ductal luminal and body cells in the ends of ducts as well as rare SMAα⁺basal cells expressed LEFTY1 (FIGS. 1D and 8G). Multiplexed pooled cell real-time PCR on sorted populations also confirmed significantly elevated Bmp7, but not Nodal, in basal populations (FIG. 8H). Hence, Applicants hypothesized that BMP7 and LEFTY1 may have different functional roles in adult mammary biology.

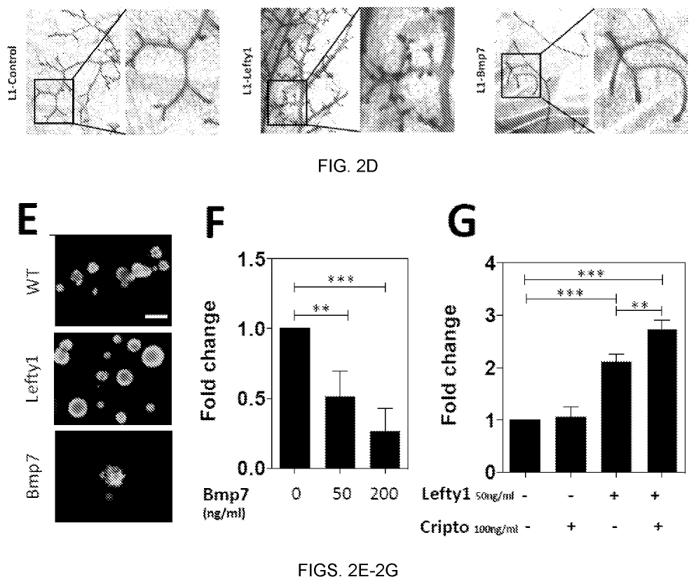

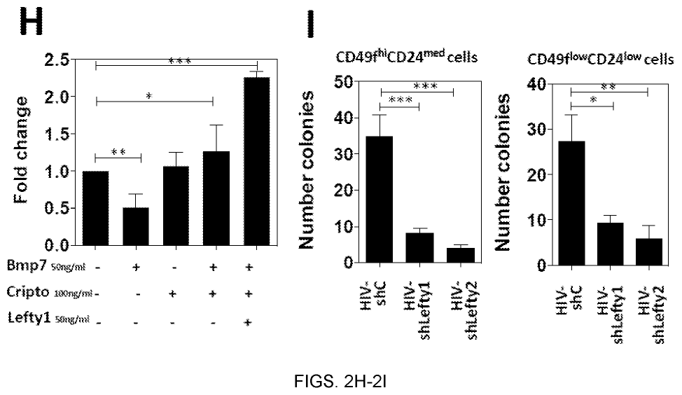

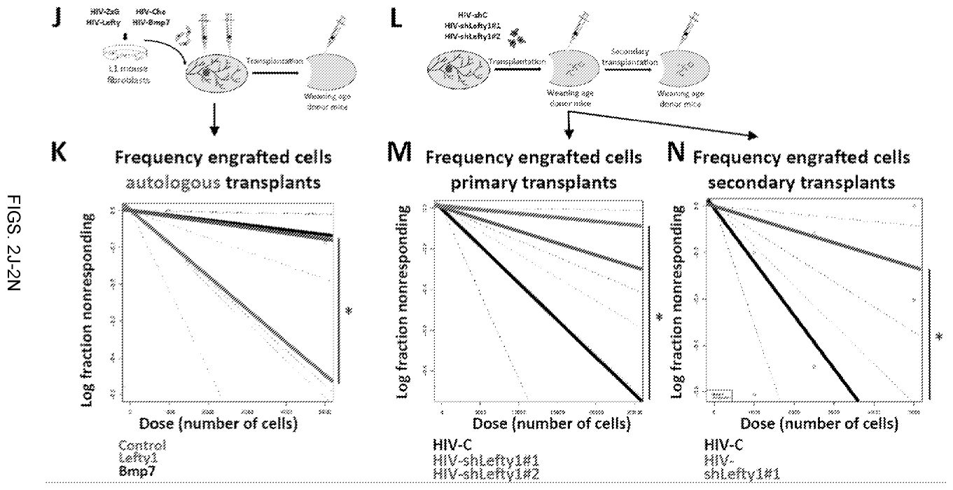

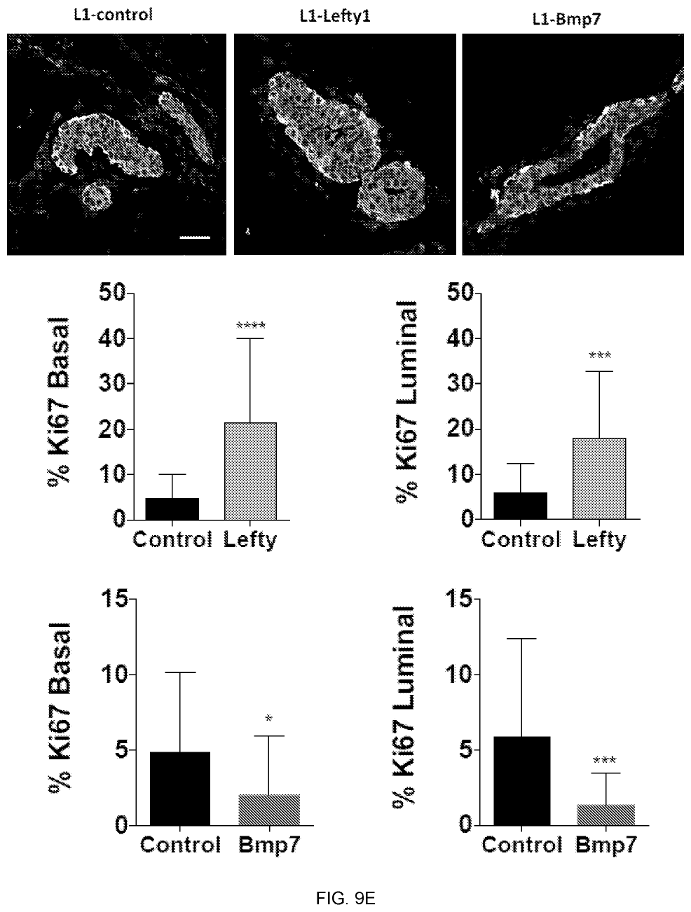

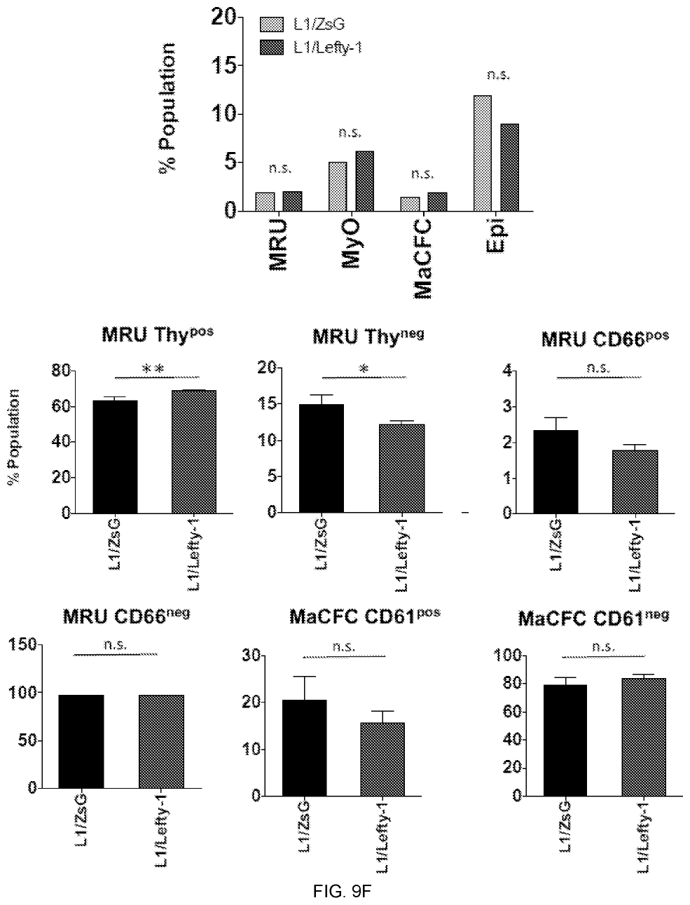

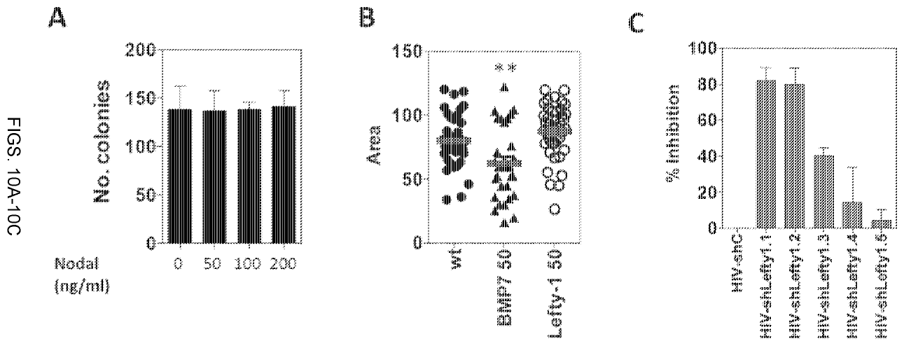

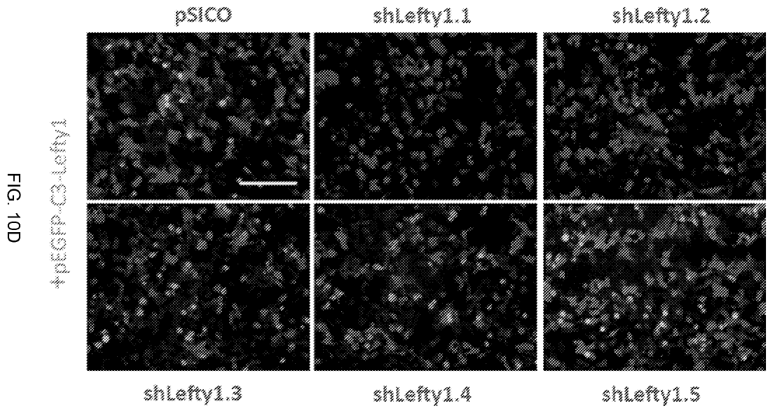

Mammary branching analysis has long been used as a model system to study key paracrine factors that regulating mammary gland function (Daniel et al., 1989; Visser et al., 1981). The role of TGFβ, CRIPTO-1 and BMP family members, including BMP7, in mammary gland development and ductal morphogenesis has been extensively explored (Inman et al., 2015; Kahata et al., 2018; Kenney N, 1997; Kowanetz et al., 2004; Moses and Barcellos-Hoff, 2011). To investigate potential functional differences between LEFTY1 and BMP7, which are expressed by different mammary gland cell populations. Applicants developed a novel system in which non-autologous mammary fibroblast cells are engineered to secrete either LEFTY1 or BMP7 and then transplanted into recipient mammary tissue (FIGS. 2A and 9A-B). Similar to Cripto-/transgenic mice (Wechselberger et al., 2001), Applicants found that secreted LEFTY1 caused a significant localized increase in the extent and number of mammary tree branches (p=0.0005) (FIGS. 2B, 2D and 9C) whereas secreted BMP7 decreased these morphological features (p=0.01) (FIGS. 2C-D and 9D). Specifically, LEFTY1 promoted the proliferation of basal and luminal cells and BMP7 inhibited the proliferation in both compartments but more dramatically in the cells of the luminal compartment, as observed by Ki67 staining (FIG. 9E). In addition, exogenous LEFTY1 stimulated an increase in endogenous Thy-1^{pos}24^{med}CD49f^{hi} cells, a phenotype that Applicants have previously shown to be enriched for long-term mammary cells (Lobo et al., 2018) (FIG. 9F). Interestingly, Applicants did not observe a similar effect on multipotent or luminal progenitor cell compartments (FIG. 9F), suggesting that secreted LEFTY1 affects SMAD signaling in mammary stem cells.

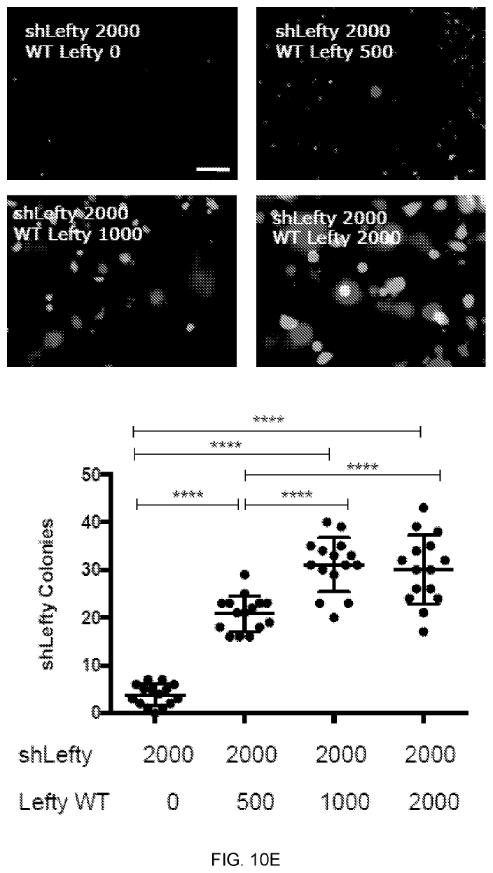

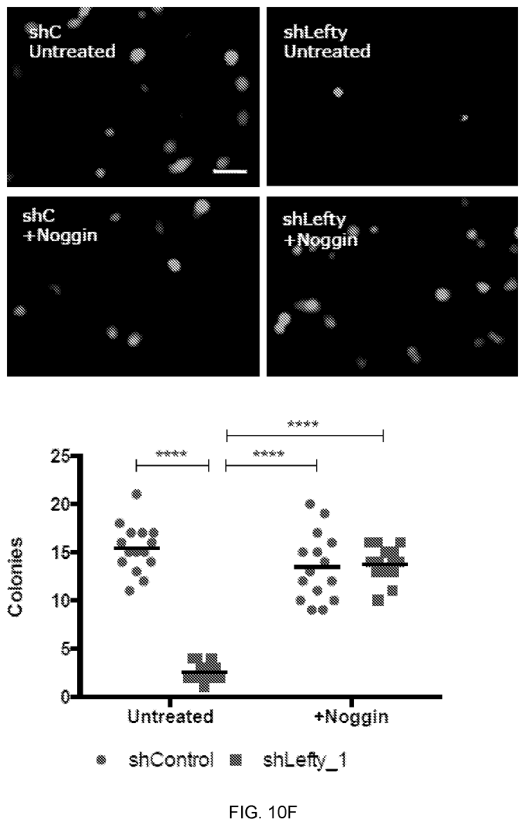

LEFTY1 promotes and BMP7 reduces the long-term proliferative potential of mammary progenitor cells. Branching phenotypes in mammary tissue have also been linked to changes in the function and frequency of long-term mammary epithelial cells (Scheele et al., 2017). Mammary epithelial cells with long-term proliferative capacity reside in the basal layer of ducts and mediate branching phenotypes in vivo (Shackleton et al., 2006). Since LEFTY1 and BMP7 were able to differentially affect the branching phenotype of mammary glands, Applicants hypothesized that the LEFTY1/BMP7 signaling axis may affect mammary progenitor proliferation in vitro. Applicants exposed FACS-sorted CD49f^{hi}CD24^{med} mammary epithelial progenitor cells to NODAL, LEFTY1, BMP7 and CRIPTO-1 in a modified Wnt-dependent in vitro organoid culture system that was optimized for progenitor cell propagation (Jamieson et al., 2017; Zeng and Nusse, 2010). NODAL had no effect on organoid formation in Applicants' culture system (FIG. 10A). Consistent with Applicants' in vivo branching phenotype results, BMP7 significantly decreased mammary organoid frequency and size in a dose-dependent fashion (p<0.0001) (FIGS. 2E-F and 10B). Conversely, LEFTY1 increased the number of organoids (p<0.0001) but not their size (FIGS. 2E, 2G and 10B), whereas soluble CRIPTO-1, like NODAL, did not have a significant effect on its own (FIG. 2G). However, soluble CRIPTO-1 enhanced LEFTY1's effect of increasing organoid formation (p<0.01) (FIG. 2G). Interestingly, the combination of LEFTY1/CRIPTO-1 was able to rescue the suppressive effect of BMP7 on organoid formation (FIG. 2H), suggesting that LEFTY1 and BMP7 may exert their effects, in part, through a common receptor. This concept is supported by LEFTY1's similar role in preventing NODAL binding to its cognate receptors (Chen and Shen, 2004). In organoid cultures, the progenitor basal cells give rise to multicellular mixed phenotype colonies. To determine if LEFTY1 was required for organoid formation, Applicants genetically inhibited Lefty/using shRNAs that significantly reduced Lefty/expression (FIGS. 10C-D). Remarkably, when Lefty1 was knocked down, there was a significant decline in the formation of organoids in long-term proliferative basal cells (CD49f^{hi}CD24^{med}) and progenitor myoepithelial cells (CD49f^{low}CD24^{low}) (p<0.001 and p<0.01, respectively) (FIG. 21), demonstrating that autocrine LEFTY1 is necessary for organoid formation by progenitor mammary gland basal cells. Taken together, Applicants' data demonstrated that autocrine and paracrine LEFTY1 promoted the proliferation of progenitor mammary cells by antagonizing the growth suppressive effect of BMP7. Furthermore, while Lefty1 deficient mammary CD49f^{hi}CD24^{med} cells do not robustly proliferate in vitro, this effect can be rescued by adding Lefty1 competent cells (FIG. 10E). Also, the addition of NOGGIN, an inhibitor of BMP signaling, to the organoid cultures ameliorated the suppressive effects of knocking down Lefty/(FIG. 10F), suggesting that LEFTY1 mediated its growth-inducing effects through suppression of BMP signaling. Hence, Applicants pursued the specific effects of LEFTY1 and BMP7 on the proliferation of mammary cells in vivo.

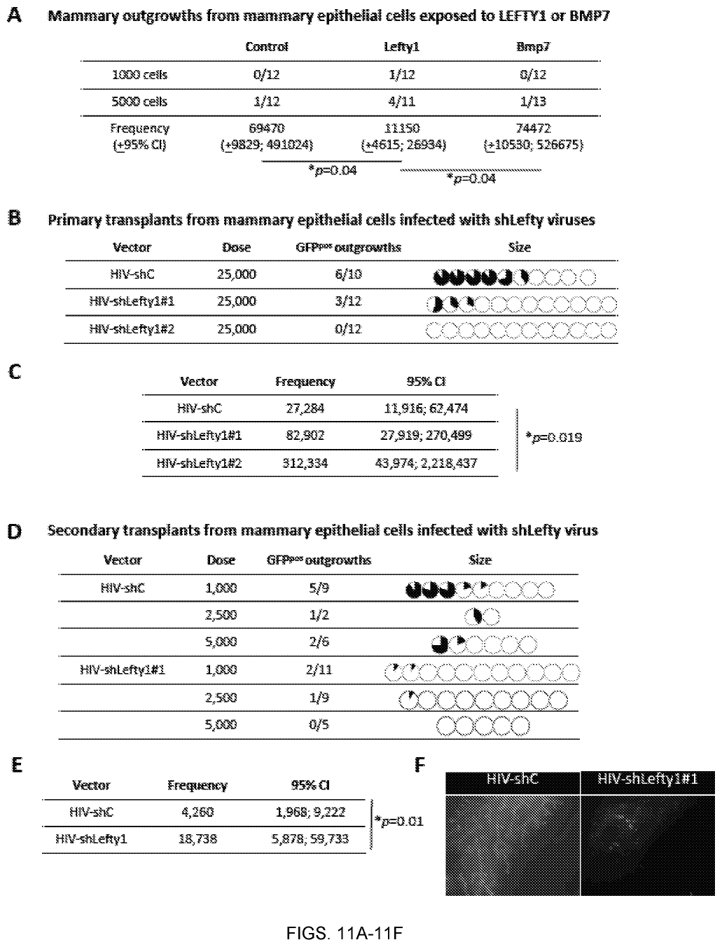

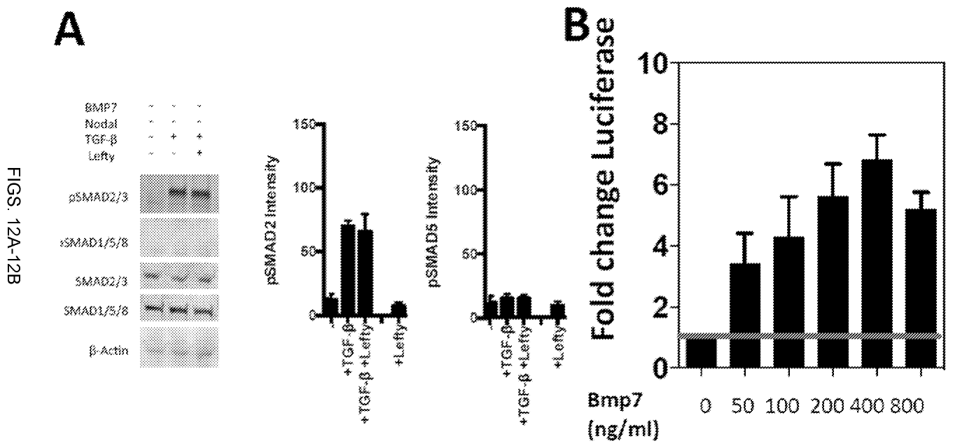

Alteration of secreted signaling pathways such as Wnt, Hedgehog and Notch results in branching phenotypes that may be linked to the frequency of cells that can be transplanted and originate mammary ducts in recipient mice (Lu and Werb, 2008). Therefore, Applicants serially transplanted endogenous mammary epithelial cells that had been exposed to LEFTY1 or BMP7 for ˜15 days in vivo and assessed their ductal outgrowth formation potential upon transplantation into the cleared fat pads of recipient mice (FIG. 2J). Applicants' data showed that transient exposure to LEFTY1 significantly increased the frequency of ductal outgrowths (p=0.04) whereas BMP7 exposure did not affect mammary outgrowth formation (FIGS. 2K and 11A). Loss-of-function genetic knockdown of Lefty1 in wild-type mammary epithelial cells significantly reduced their frequency of ductal outgrowth formation in vivo (p=0.019) (FIGS. 2L, 2M and 11B-C). This effect was more pronounced upon secondary transplantation, an assay that measures the long-term proliferative potential of progenitor cells (p=0.01) (FIGS. 2L, 2N and 11D-E). Visualization of the secondary transplants showed the severely restricted ductal formation of shLefty1-infected cells (FIG. 11F). These results demonstrated that at least a subset of mammary epithelial cells with long-term repopulation potential require LEFTY1 to facilitate the formation of mammary glands. Furthermore, a transient exposure of cells to LEFTY1 enhanced the serial transplantation capability of immature cells, suggesting that LEFTY1 may be a component of long-term proliferation.

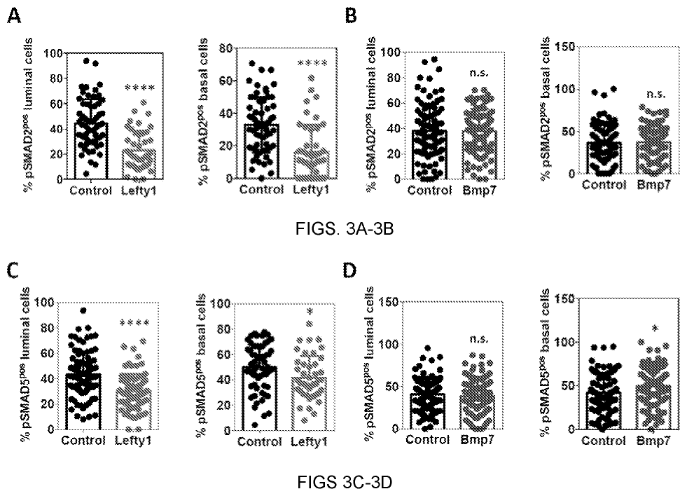

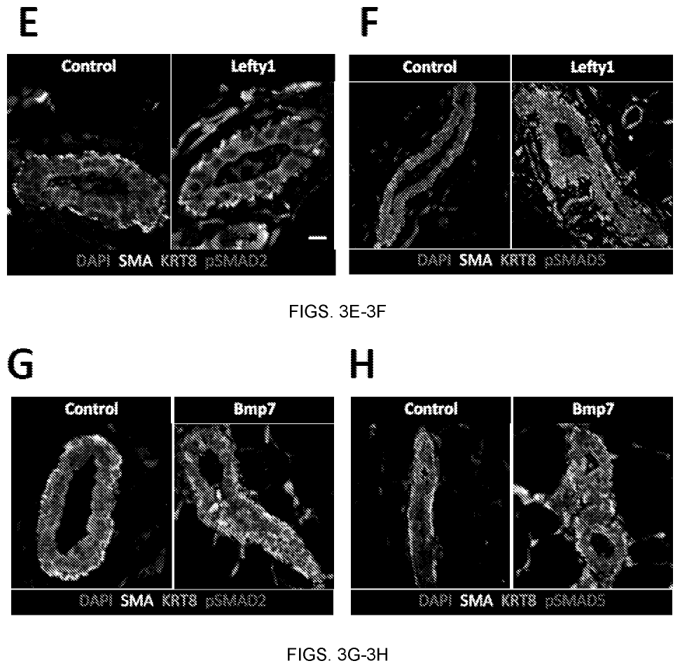

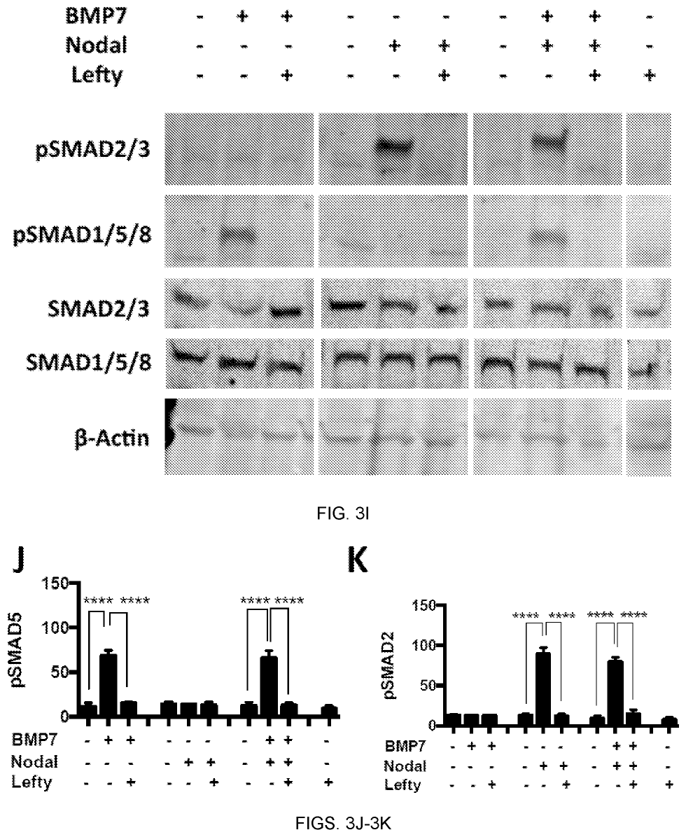

LEFTY1 simultaneously suppresses SMAD2 and SMAD5 in basal cells. Recently, it has been shown that chemical inhibition of SMAD2 and SMAD5 can confer long-term proliferative capacity to a variety of epithelial cell types in vitro (Mou et al., 2016). NODAL and BMP signaling are mediated through SMAD2/3 or SMAD1/5/8 proteins, respectively (Miyazawa et al., 2002). In engineered 293T cells, LEFTY1 is known to inhibit NODAL/SMAD2 signaling (Cheng et al., 2004) and BMP4-mediated SMAD5 phosphorylation in P19 embryonal carcinoma cell line cells (Ulloa and Tabibzadeh, 2001). Thus, Applicants hypothesized that LEFTY1 induces proliferation in mammary epithelial cells by inhibiting both SMAD2 and SMAD5. First, Applicants characterized how ectopic LEFTY1 and BMP7 affected phosphorylated SMAD2 and SMAD5 expression in ductal mammary epithelial compartments in vivo using Applicants' non-autologous fibroblast transplant system (FIG. 2A). Consistent with the known function of LEFTY1 (Kim et al., 2014), ectopic LEFTY1 exposure caused a significant decrease in pSMAD2 expression in basal and luminal compartments (FIGS. 3A and 3E), whereas BMP7 had no effect on pSMAD2 (FIGS. 3B and 3G). Strikingly, LEFTY1 exposure also caused a significant decrease in pSMAD5 in both basal and luminal ductal compartments (FIGS. 3C and 3F). BMP7 caused a significant increase of pSMAD5 only in the basal cell compartment (FIGS. 3D and 3H). Applicants confirmed that LEFTY1 not only represses NODAL-activated pSMAD2/3, but also opposes BMP7-activated pSMAD5 in the mammary epithelial COMMA-D cell line model (FIGS. 3I-K). In this specific context, Applicants did not observe an attenuation of pSMAD2/3 signaling by LEFTY1 upon addition of TGFB1 ligand (FIG. 12A). However, TGFβ signaling is often context dependent, with potent TGFB1 effects on mammary development described using in vivo models (Moses and Barcellos-Hoff, 2011). LEFTY1 reduction of BMP7 signaling was also validated with mammary fibroblasts that expressed the BRE luciferase reporter construct (FIGS. 12B-D). Applicants' studies confirmed that LEFTY1 suppresses both pSMAD2 and pSMAD5 in the mammary basal epithelial compartment, where immature cells with the highest proliferative potential are thought to reside (Shackleton et al., 2006; Stingl et al., 2006).

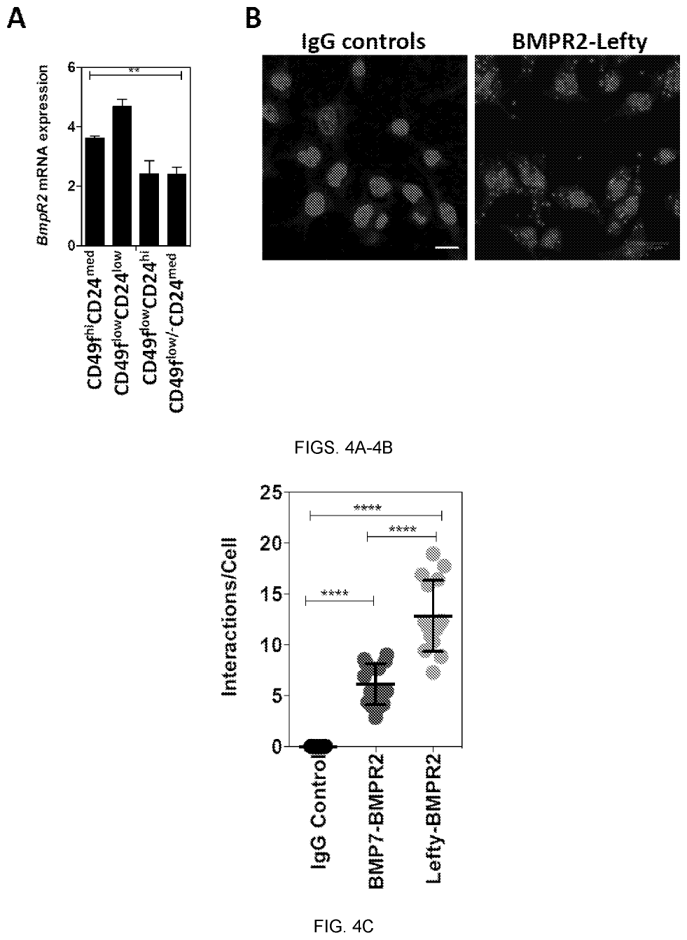

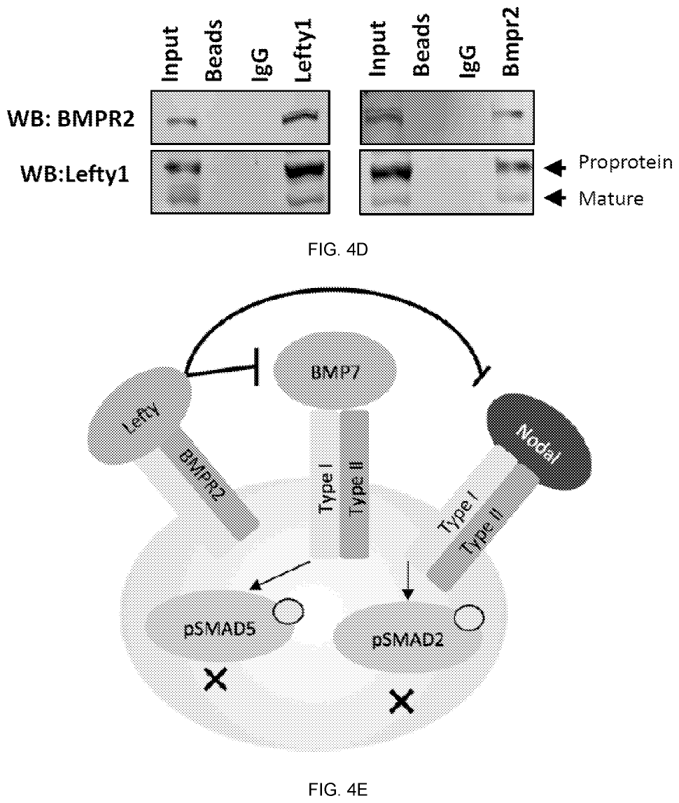

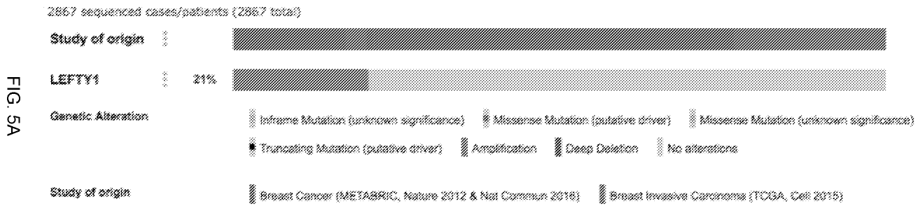

LEFTY1 directly binds to the BMP receptor BMPR2. Consistent with previous studies (Meno et al., 1997; Ulloa and Tabibzadeh, 2001), Applicants' data suggested that LEFTY1 attenuates BMP signaling. LEFTY1 has previously been shown to partially inhibit the BMP4-mediated phosphorylation of SMAD5 through an unknown mechanism (Ulloa and Tabibzadeh, 2001). Although BMPs are known to be redundant in numerous instances, BMP2, BMP4 and BMP7 have non-overlapping phenotypes in limb patterning and differentially use receptors to induce osteoblastic differentiation of mesenchymal stem cells (Bandyopadhyay et al., 2006; Lavery et al., 2008). Applicants postulated that LEFTY1's inhibition of BMP7 signaling could be due to LEFTY1 directly binding a BMP receptor. To identify potential candidate target receptors that could bind to both LEFTY1 and BMP7. Applicants first performed a pooled real-time PCR expression screen for specific type I and type II TGFβ receptor components that are expressed on distinct mammary populations using the expression of KRT14 and KRT8 as basal and luminal lineage identification markers, respectively (FIGS. 12E-F). Applicants looked for receptors with high expression levels in basal cells compared to luminal cells, since Applicants observed LEFTY1-mediated SMAD2 and SMAD5 suppression in the basal compartment in vivo. Applicants found that basal populations expressed significantly more Bmpr2 (p=0.0011) and Acvr2a (p=0.0083) compared to luminal populations (FIGS. 4A and 12E). In contrast, Bmpr1a (p=0.019) and Bmpr1b (p<0.0001) were expressed at significantly higher levels in luminal populations compared to basal populations (FIG. 12E). Applicants did not observe a statistical difference in Acvr1. Acvr1b, Acvr1e or Acvr2b expression between luminal and basal populations (FIG. 12E). Applicants next analyzed if there was a direct interaction between LEFTY1 and selected receptors using a proximity ligation assay. Applicants used the BMP7-BMPR2 interaction as a positive control and the interaction of LEFTY1 with LRP6 (another stem cell related receptor), or IgG as negative controls (FIG. 12G). Applicants quantified more interactions between LEFTY1 and BMPR2 than the well-characterized interaction of BMPR2 with BMP7 (p<0.0001) (FIGS. 4B-C). Furthermore, Applicants were also able to detect the binding of LEFTY1 to BMPR2 and vice versa in LEFTY1 and BMPR2 co-immunoprecipitation assays (FIG. 4D). Given that BMPR2 is upstream of pSMAD2 and pSMAD5 and is shared by both NODAL and BMP7 (Aykul et al., 2015; Yu et al., 2005), Applicants found it likely that BMPR2 serves as a common BMP Type II receptor that mediates the BMP7/LEFTY1 axis (FIG. 4E). Upon addition of a LEFTY1 blocking peptide in triple negative MDA-MB-231 breast cancer cells in vitro, BMP7-BMPR2 interactions and induction of pSMAD5 increased in a dose-dependent manner, indicating a potential mechanism by which LEFTY1 modulates BMP7 signaling (FIGS. 12H-I).

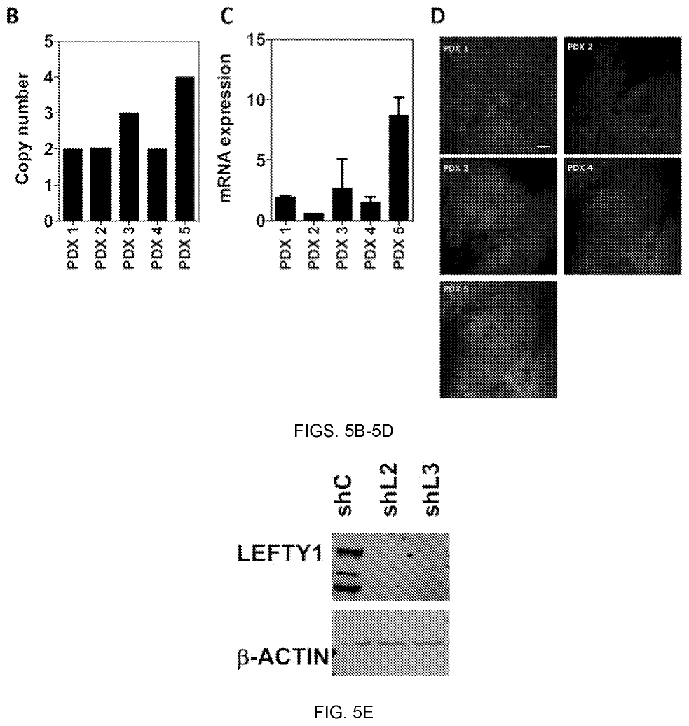

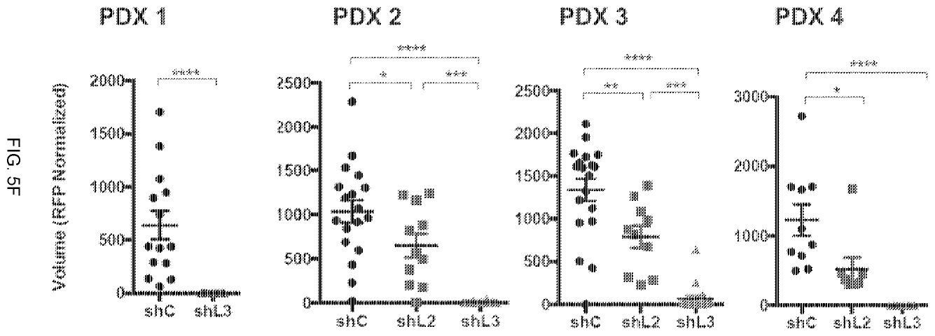

Inhibition of LEFTY1 abrogates the proliferation of breast cancer cells in vivo. Nodal expression has been shown to mediate both the proliferation and stem cell associated properties of some breast cancer cell lines (Gong et al., 2017; Kirsammer et al., 2014). Although the primary role of LEFTY1 is thought to be the suppression of NODAL, LEFTY1 expression in breast cancer remains unexplored. Therefore, Applicants probed the TCGA (Cerami et al., 2012; Gao et al., 2013) and METABRIC (Curtis et al., 2012) genomic cancer databases for alterations in LEFTY1 in human breast cancer samples. Surprisingly, Applicants found that LEFTY1 was amplified in ˜20% of human breast cancer samples across both datasets (FIG. 5A). Using well-characterized patient-derived xenograft (PDX) models established in Applicants' lab (Table 1), Applicants detected an increase of LEFTY1 copy number of in 2 out of 5 models (FIG. 5B) and confirmed detectable LEFTY1 mRNA expression levels in all 5 of Applicants' models (FIG. 5C). These results were confirmed by immuno-fluorescent staining (FIG. 5D).

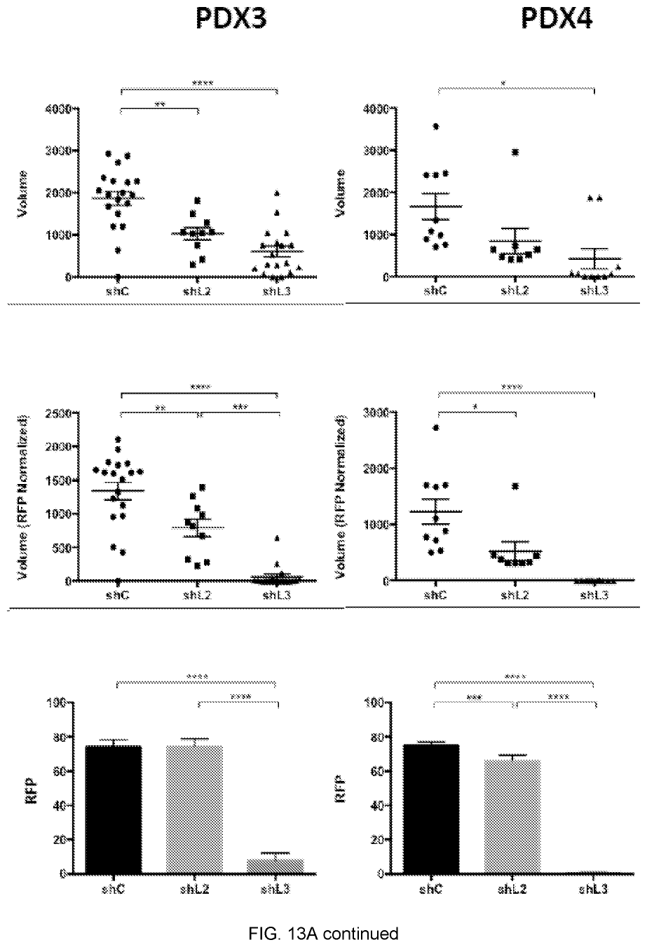

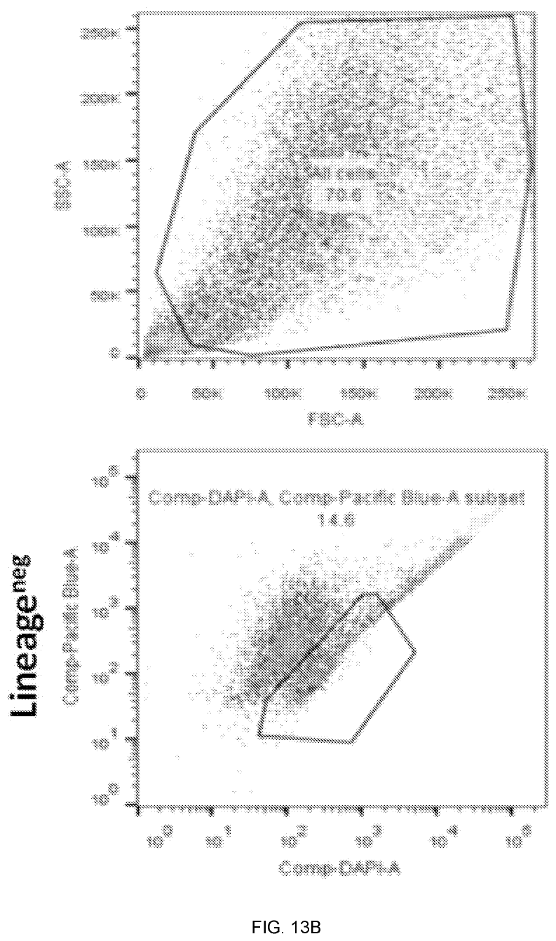

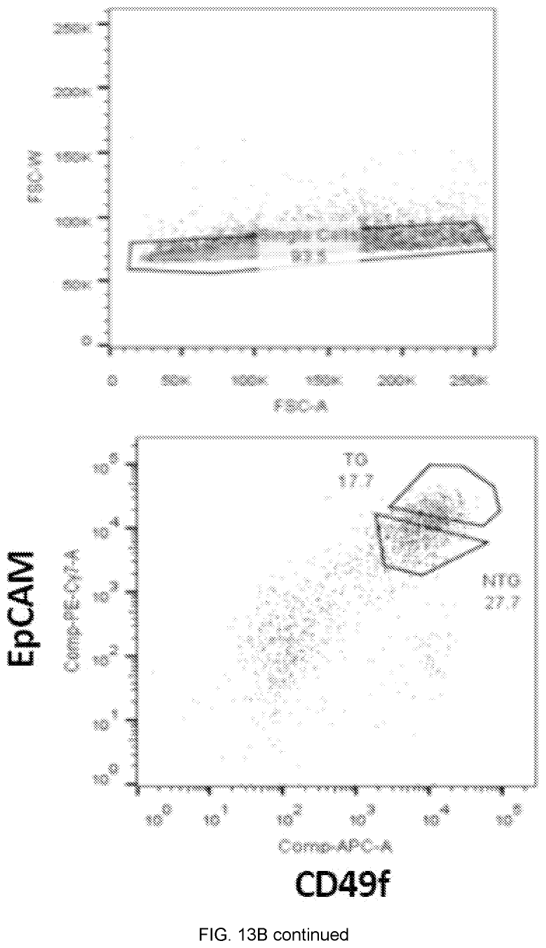

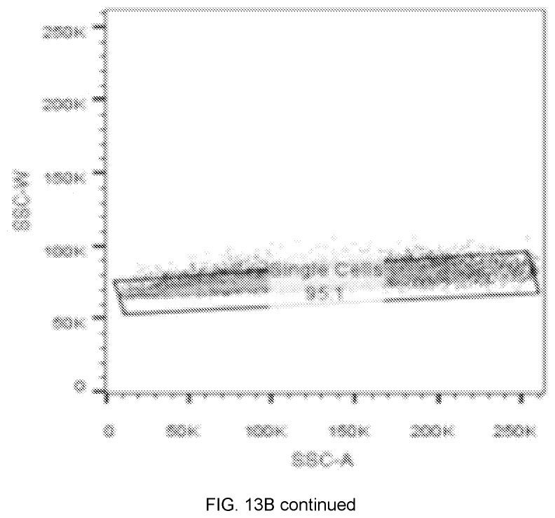

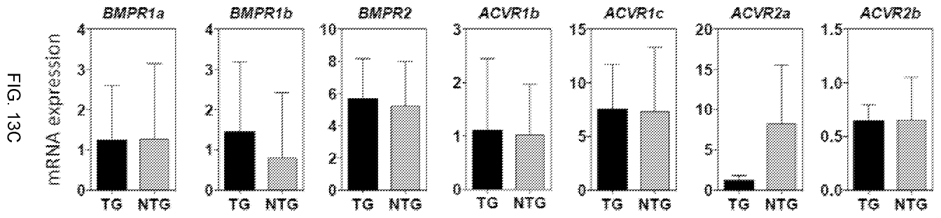

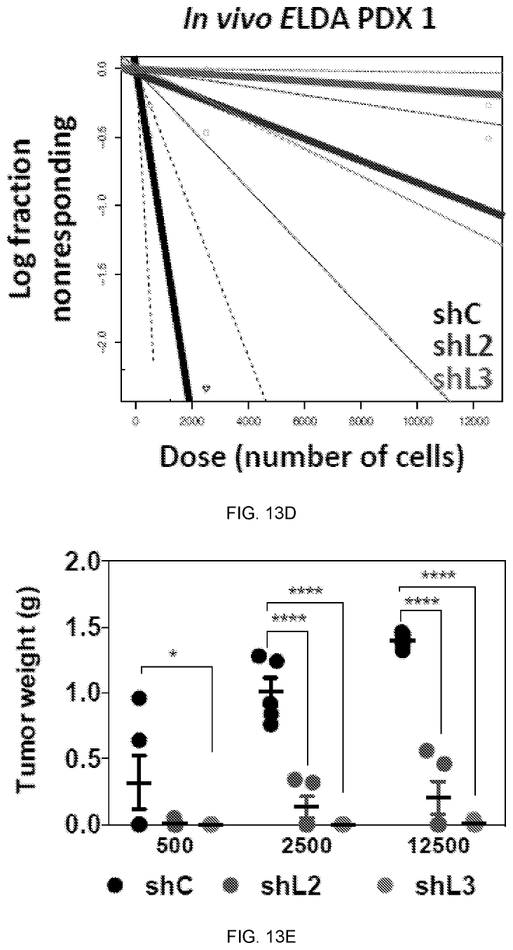

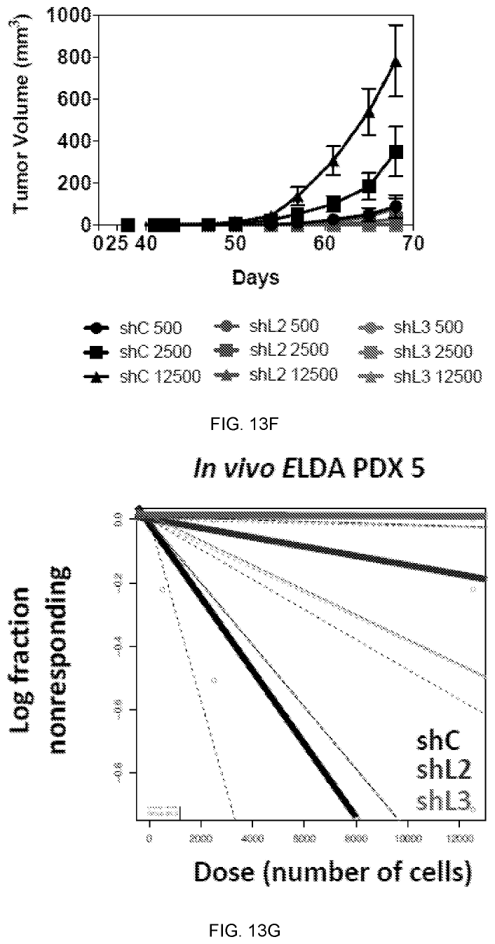

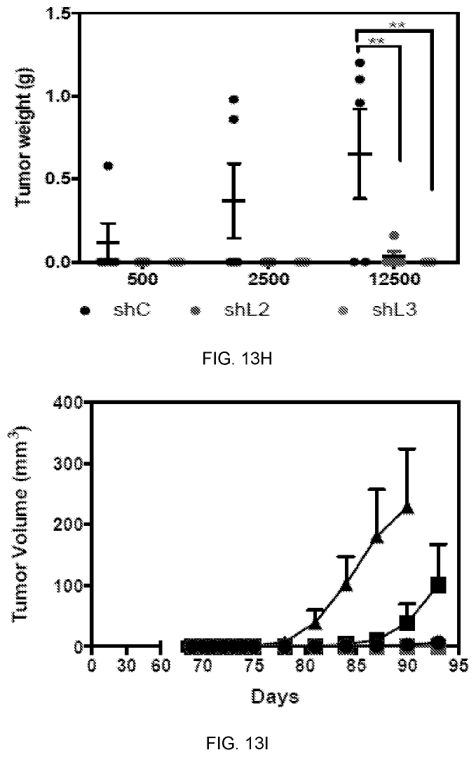

Mechanisms that regulate long-term proliferation have increasingly been implicated as also playing a role in the growth and maintenance of cancer. Indeed, successful therapies have been brought forward that inhibit long-term proliferation pathways, such as the Hedgehog pathway (vismodegib), in solid tumors (Marcucci et al., 2016). Based upon Applicants' functional studies with normal mouse epithelium and the high conservation of long-term proliferative pathways between mouse and human, Applicants performed loss-of-function studies to determine if Applicants' breast PDX models' growth was driven by autocrine LEFTY1 production. Strikingly, Applicants found that genetic knock-down of LEFTY1 (FIG. 5E) in 4 of Applicants' PDX models caused significant impairment of tumor progression or completely inhibited in vivo tumor growth in all of the models that Applicants tested (p<0.0001 for all, ANOVA, multiple-comparison values are shown for individual comparisons within ANOVA) (FIGS. 5F and 13A). Therefore, Applicants' data indicates that at least some tumorigenic human breast tumor cells require LEFTY1 expression to form tumors.

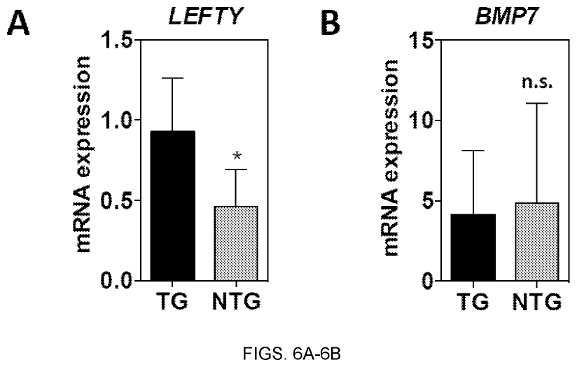

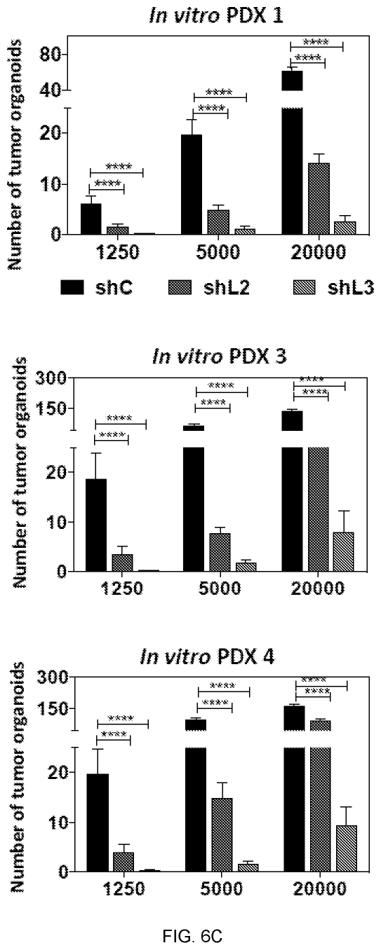

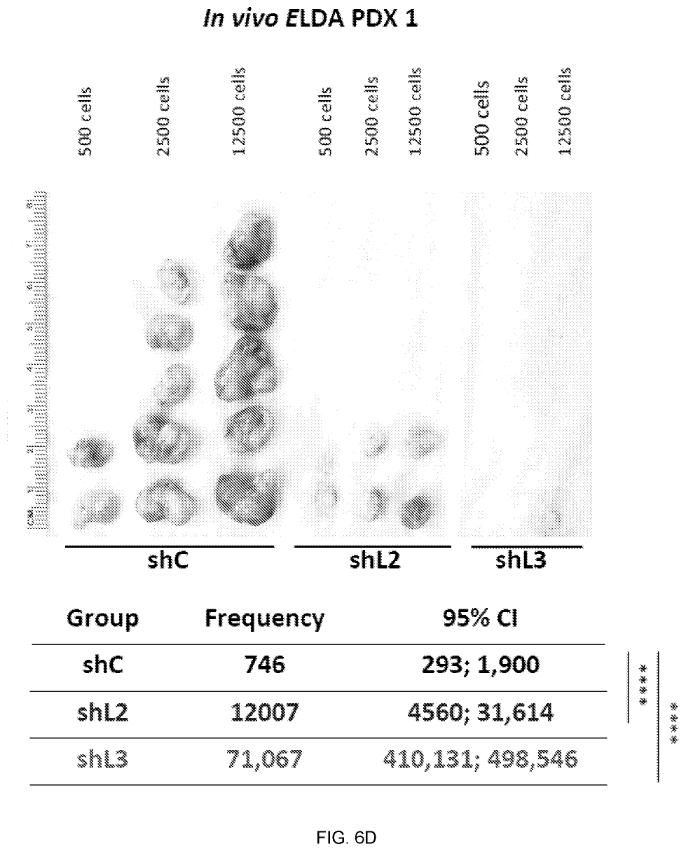

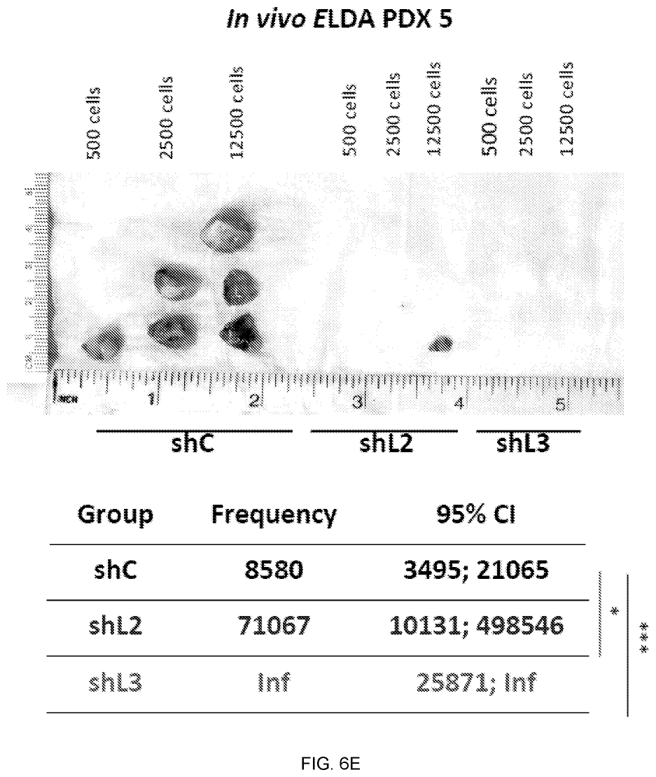

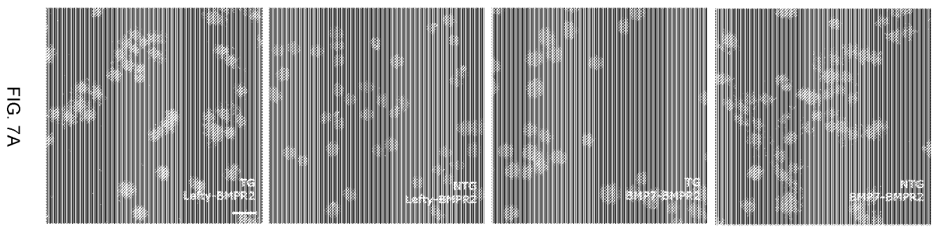

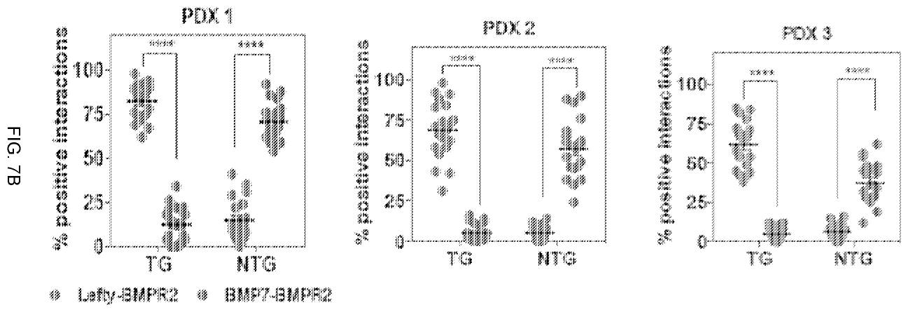

Autocrine LEFTY1 is necessary for the proliferation of tumor initiating breast cancer cells. Many studies have now demonstrated that there is a proliferative cellular hierarchy in at least some breast tumors, wherein cells with long-term proliferative capacity are resistant to radiation and chemotherapy (Lobo et al., 2007). Applicants' group first identified a phenotypic marker profile that successfully enriched for long-term tumorigenic cells from breast tumors (Al-Hajj et al . . . 2003). Applicants used their breast cancer PDX models to further investigate if tumorigenic cells (TG, enriched by the CD49f⁺EpCAM⁺phenotype, FIG. 13B) (Sikandar et al., 2017) produced autocrine LEFTY1. Applicants observed that TGs expressed significantly higher LEFTY1 compared to non-TGs (NTG, defined by non-CD49f⁺EpCAM⁺) in 4/5 PDX models (FIG. 6A). Applicants did not observe significant differences between Applicants' PDX models for the expression of BMP7 (FIG. 6B) or other type I and II TGFβ receptors that were differentially expressed by luminal and basal normal mouse mammary populations (FIG. 13C).

Since the TG compartments in most of Applicants' models expressed LEFTY1 and relied on LEFTY1 for proliferation. Applicants pursued loss-of-function studies in vitro and quantitated the frequency of cells with proliferative capacity upon knock-down in vivo. In vitro colony formation assays performed in a limiting dilution fashion showed that the growth of cells isolated from 4 breast cancer PDX models relied on autocrine LEFTY1 expression for tumor formation (FIG. 6C). In vivo, genetic knock-down of LEFTY1 significantly reduced the frequency of TGs by 16 and 8-fold in PDX1 and PDX5, respectively (FIGS. 6D-E, 13D and 13G) (shC vs shL2; p=2.27×10e⁻⁵; shC vs shL3; p-2.47×10e⁻⁸). In addition to the altered frequency of tumor initiating cells, tumor weight was also significantly reduced, underscoring the impact on tumor growth (FIGS. 13E and 13H). The rate of tumor growth was severely attenuated, indicating that LEFTY1 is indeed important for proliferation of at least a subset of tumor-initiating cells in the tumorigenic compartment (FIGS. 13F and 131). Applicants' functional data validated that at least a subset of tumor initiating breast cancer cells rely on autocrine LEFTY1 for engraftment and proliferation and that LEFTY1 knockdown has a profound impact on tumorigenicity.

LEFTY1 binds to BMPR2 and is a concomitant SMAD2 and SMAD5 inhibitor in tumor initiating cells. Since Applicants observed that LEFTY1 induces both the long-term proliferation in a subset of normal mammary epithelial cells and the simultaneous inhibition of pSMAD2 and pSMAD5, Applicants hypothesized that LEFTY1 affect TGs in Applicants' PDXs by a similar mechanism. Thus, Applicants analyzed the binding of LEFTY1 or BMP7 to BMPR2 in the TG and NTG subpopulations that Applicants isolated from different PDX models. In agreement with Applicants' observations in the normal mouse mammary gland. Applicants observed significantly more LEFTY-BMPR2 interactions on TGs compared to NTGs in 3/3 PDXs (FIGS. 7A-B). Conversely, BMP7-BMPR2 interactions were more prevalent on NTGs compared to TGs. pSMAD2 and pSMAD5 were also significantly repressed in TG compartments compared to NTG compartments in all the 3 PDXs (FIGS. 7C-D). Next. Applicants isolated TGs and NTGs from Applicants' PDXs, serum starved them overnight and treated with exogenous LEFTY1 and/or BMP7. Strikingly, only the NTGs responded to BMP7 stimulation and activated pSMAD5, while the TGs (expressing endogenous LEFTY1) did not (FIG. 7E). Similarly, in MDA-MB-231 breast cancer cells, addition of BMP7 induced pSMAD5, which could be abolished upon concomitant addition of LEFTY1 (FIG. 7F). Taken together with the functional studies, Applicants' results indicated that TGs overexpress LEFTY1 to concomitantly suppress SMAD2 and SMAD5 in order to promote their long-term proliferation (FIG. 7G).

Proper mammary gland development and maintenance relies on autocrine and paracrine signaling networks to orchestrate homeostasis (Visvader and Stingl, 2014). Applicants' data indicates that secretion of LEFTY1 and BMP7 from distinct cellular compartments in normal mammary epithelium constitutes a novel paracrine regulatory system. The work presented here demonstrates novel functions for LEFTY1 as a ligand that promotes self-renewal in mammary gland epithelium. Applicants also show that LEFTY1 induces the clonogenicity of at least some tumorigenic breast cancer cells, in part, by binding to the BMPR2 receptor and suppressing BMP7 induced SMAD5 activation. LEFTY1, acts as a physiological SMAD2 and SMAD5 inhibitor via its interactions with NODAL and BMPR2, respectively, to promote the long-term growth of basal mammary epithelial and breast cancer cells in vivo. Thus, LEFTY1 is a physiological “dual-SMAD inhibitor” protein that had only been previously hypothesized to exist (Chambers et al., 2009). Previous description of dual-SMAD inhibition via pharmacologic inhibitors demonstrated that this type of mechanism is used by the long-term epithelial basal cells from a wide variety of epithelial tissue types (Mou et al., 2016). The notion that the LEFTY1/BMP7 pathway may regulate different stem cell compartments is consistent with the phenotypes elicited from Lefty-1″ mice, which show heterotaxic defects in visceral organs such as lung, heart and liver (Meno et al., 1998).

BMP signaling plays a crucial role in normal development, including the mammary gland (Chapellier et al., 2015; Dituri et al., 2019; Hens et al., 2007; Hiremath and Wysolmerski, 2013; Jung et al., 2019; Prasad et al., 2019; Tan et al., 2015; Zinski et al., 2018). BMP ligands signal, at least in part, though BMPR2 to transduce tumor suppressive signaling in mammary epithelium (Owens et al., 2012). Here, Applicants show that BMP7 is secreted from the basal compartment of mammary epithelium and reduces the proliferation of progenitor cells. BMP7 secretion did not affect pSMAD5 expression in the luminal compartment; therefore, ductal branching morphogenesis is likely linked to the regulation of progenitor proliferation in the basal compartment. This compartmentalized expression is consistent with ΔNp63, a basal cell expressed transcription factor shown to promote mammary self-renewal (Chakrabarti et al., 2014), activating BMP signaling by inducing the expression of BMP7 (Balboni et al., 2013) as potential negative regulation loop. In some cell lines, BMP7 increases the clonogenic capacity of immortalized human mammary epithelial cells (Balboni et al., 2013), while in others BMP7 may exert an anti-proliferative effect potentially through a Tert-related mechanism (Cassar et al., 2017) or through a LMO4 autocrine feedback loop (Wang et al., 2007). Applicants' data also showed that continual BMP7 signaling was necessary to elicit its anti-proliferative effect in their models, in contrast to the durable activation of a long-term proliferative program initiated by a relatively brief LEFTY1 exposure. BMP7 and LEFTY1 both bind to BMPR2, ACVRIA and BMPR1A (Pick et al., 1999), indicating that LEFTY1's effects may extend beyond BMPR2 signaling. However, while BMP7 binds to BMPR1A, LEFTY1 had little interaction with this receptor. Applicants' data reporting that BMPR1a is upregulated in luminal mammary epithelial populations is consistent with previous reports that determined that BMPR1A is required for hormone signaling and milk production (Hens et al., 2007; Perotti et al., 2012).

The LEFTY1/BMP7 pathway has important implications for breast cancer. Numerous clinical studies are underway to evaluate therapeutics that target mechanisms that drive the long-term proliferation of tumor-initiating cells (Dragu et al., 2015). Applicants' studies to support the possibility that inhibitors of LEFTY1 may be useful for the treatment of LEFTY1-expressing breast cancers. Applicants' data with multiple triple negative PDX models demonstrates the reliance of at least some TGs on autocrine LEFTY1 production for tumor growth. Furthermore, Applicants' studies suggest that the LEFTY1-BMPR2 interaction is specific to the tumorigenic cell population in some tumors, presenting a potential biomarker to assess the effect of LEFTY1 abrogation. The relationship between LEFTY1/BMP7/BMPR2 could also lead to the development of novel anti-tumor therapies and biomarkers for breast cancer treatment.

Despite numerous studies on the role of BMP7 in breast cancer, the context-dependent functions of BMP7 remain poorly understood. BMP7 inhibits the growth and metastasis of triple negative MDA-MB-231 breast cancer cells in vivo and in vitro, but not their proliferation in vitro (Alarmo et al., 2009; Buijs et al., 2007). This was also largely true for hormone receptor positive MCF7 breast cancer cells (Ying et al., 2017), except estrogen-induced mitosis was required for a potent anti-proliferative BMP7 effect in vitro (Takahashi et al., 2008). However, another study demonstrated that BMP7 does induce senescence, growth arrest and apoptosis in MCF-7 cells (Cassar et al., 2017), with the added complexity of p53 status as a potential biomarker of efficacy (Yan and Chen, 2007). BMP7 acts as a tumor suppressor in human gastric, renal cells, lung and colorectal cancers by inducing the differentiation of tumorigenic cancer cells (Shi and Massague, 2003; Ych, 2010). Applicants' work presents the novel therapeutic hypothesis that cancers that are driven by mechanisms that suppress SMAD2 and SMAD5 signaling, such as those that rely on LEFTY1, may be susceptible to therapies that attenuate dual-SMAD inhibition.

The work presented here describes a new mechanism by which LEFTY1 acts a physiological dual-SMAD inhibitor to promote the long-term growth of immature normal and cancerous breast cells. Applicants used engineered L1 fibroblasts to study the exogenous effects of LEFTY1 protein in normal mouse mammary glands in situ. These immortal mammary fibroblast cells were irradiated before their transplantation, making their secretion of ligands finite. The use of syngeneic primary mammary fibroblasts is technically challenging due to their finite lifespan and resistance to genetic manipulation. Genetic studies for in vivo mammary fibroblast targeting are challenging due to the lack of specific markers for this population of cells. The TGFβ pathway is complex, context dependent and often involved in breast cancer actiology (Ikushima and Miyazono, 2012; Massague, 2008), so models of TGFβ signalling may be affected by specific experimental designs and the unique mutations inherent to any individual model. Although Applicants' results indicate that LEFTY1 may be a novel therapeutic breast cancer target, the studies do not address possible challenges in preclinical studies like the potential toxicities that associate with system administration of an anti-LEFTY1 agent.

Mice. C57BL/6 and NOD scid gamma (NSG) female mice were purchased from Jackson Laboratories. pCx-GFP mice were kindly provided by Dr. Weissman. All the mice used in this study were maintained at the Stanford Animal Facility in accordance with a protocol approved by the Stanford University APLAC committee. Mice were maintained in-house under aseptic sterile conditions. Mice were administered autoclaved food and water.

Cell culture. Human embryonic kidney (HEK) 293T cells, MDA-MD-157, MDA-MB-231, and 3T3-L1 mouse embryonic mammary fibroblast Applicants maintained in Dulbecco's modified Eagle's medium (DMEM) supplemented with 10% heat-inactivated fetal bovine serum (FBS), 100 μg/ml streptomycin and 100 unit/ml penicillin and 2 mM glutamine (Invitrogen). The cells were purchased from ATCC. COMMA-D beta cell line was cultured in DMEM-F12 (Invitrogen) supplemented with 2% of Fetal Bovine Serum (Hyclone), 1% PSA (Invitrogen), 10 ng/ml EGF (BD) and 5 μg/ml Insulin (Sigma), at 37 degree with 5% CO2. All cells were incubated at 5% CO2 and 37° C. None of the cell lines used are listed in the database of commonly misidentified cell lines maintained by ICLAC. Cell lines have not been authenticated but all cell lines used were passaged less than 10 times from when the original cells from the vendors were thawed.

Organoids were grown using published methods with adaptations for breast cells (Debnath et al., 2003; Rothenberg et al., 2012; Sato et al., 2009; Zeng and Nusse, 2010). Eleven thousand L1-Wnt3a irradiated cells were mixed with growth factor-reduced Matrigel and plated in low attachment 96-well plates. Sorted cells were mixed in breast organoid media (advanced DMEM/F12+10 mM Hepes+1× Glutamax+10% FBS+1× ITES+10 uM Y-27632+R-spondin3 (250 ng/ml; R&D)+EGF (10 ng/ml; R&D)+Noggin (100 ng/ml; R&D) supplemented with 2.5% of growth factor reduced Matrigel. When the organoids were grown in the presence of LEFTY-1 (R&D Systems 994-LF), BMP7 (R&D Systems 5666-BP), CRIPTO-1 (R&D Systems 1538-CR) and/or NODAL (R&D Systems 1315-ND), Noggin was excluded from the culture media. Organoid cells were routinely co-cultured with L1-cells expressing Wnt3a. Organoids were grown in humidified tissue culture incubators at 37° C. in 5% CO2 and were supplemented every other day with fresh media. Organoids were defined as viable multicellular structures.

For the co-culture experiments, 2000 Lefty deficient mammary CD49f^{hi}CD24^{med} cells/well were cultured in vitro to form organoids in a 96 well plate, along with Lefty competent cells in increasing doses of 0, 500, 1000, 2000 cells (5 replicates, n=3).

To evaluate BMP7 inhibition, mammary CD49f^{hi}CD24^{med} epithelial cells already infected with shControl or shLefty were cultured in vitro to form organoids in a 96 well plate, and treated with 20 ng/ml Noggin (media replaced every 2 days) for 21 days.

For evaluating SMAD phosphorylation COMMA-D cells were serum starved 16 hours and blocked with LEFTY1 (200 ng/ml) for 1 hour and stimulated with BMP7 (50 ng/ml) and Nodal (50 ng/ml) for 30 minutes in serum free media. Breast cancer cell line MDA-MB-231 were serum starved 16 hours and treated with LEFTY1 (200 ng/ml) and/or BMP7 (50 ng/ml) for 30 minutes in serum free media. From PDX1, TGs and NTGs were serum starved overnight and treated with exogenous LEFTY1 (200 ng/ml) and/or BMP7 (50 ng/ml) for 30 minutes in serum free media. Further analysis was performed by immunoblotting.

In Lefty Blocking Peptide (LBP) studies, MDA-MB-231 cells were pre-incubated with LBP in serum free media for 1 hour at increasing doses of 1×, 2×, 4×, 8×, 16×, 64× and 256× where x=25 ng·mL⁻¹ and treated with LEFTY1 (200 ng/ml) and/or BMP7 (50 ng/ml) for 30 minutes.

Production of engineered 3T3-L1 cells. 3T3-L1 cells were infected with HIV-ZsG, HIV-Lefty1-ZsG, HIV-Che and HIV-Bmp7-Che virus at 50 moi. Three to five days later, GFP+ cells were isolated using flow cytometry and the cells were expanded in vitro and frozen stocks prepared using 10% glycerol solution with basal media. Fresh cell stocks were prepared every 6 months.

Tumor injection and PDX establishment. Mice were sedated using isoflurane. PDXs were established as previously described (Al-Hajj et al., 2003). For single cell suspension injections, the cell mixture (1:1 staining media: Matrigel; final volume 100 μl per injection) was injected near the mammary fat pads of the NSG female mice using a 23-gauge, 1-inch needle. When ERPS tumors were engrafted, slow-release ER pellets were placed into the mice. Vetbond was used to close the injection site and the subcutaneous incision to place the pellet. Mice were observed weekly for tumor formation. Tumor growth in live mice were documented twice per week using once palpable and recorded as Length (L)×Width (W) using Vernier callipers. Volume was calculated as L*W*W/2. At the completion of study, final tumor volumes along with tumor weights were recorded. A summary of the different PDX models is described in Table 1.

For the limiting dilution assays, 500, 2500, 12500 cells from either shC, shL2 or shL3 infected PDX were injected into the mammary fat pads of NGS mice (n=5 per condition), 1:1 media: Matrigel; final volume 100 μl per injection.

Immunofluorescence. Mammary glands and breast cancer PDXs were fixed in formalin and embedded in paraffin for immunostaining. Sections were de-paraffinized, dehydrated and microwaved for 10 min at 95° C. in Tris-EDTA (0.01-0.001 M; pH 9) for antigen retrieval. Tissue sections were incubated o/n at 4° C. with the indicated primary antibodies diluted in TBS+5% BSA (Table 2). For the analysis of LEFTY1 and BMP7 in the terminal end of ducts, frozen mammary sections were used. In this case, the glands were immediately placed into OCT compound (Tissue Tek) and frozen using a liquid N₂ cooled isopentane bath. Blocks were then transferred to −80 C until ready for sectioning. Dried sections were then stained with primary antibodies diluted in PBS+4% FBS+0.1% TritonX-100. The primary antibodies used are described in Table 2. Samples were incubated with Alexa Flour conjugated secondary antibodies (Invitrogen) at 1:500 in TBS+5% BSA for the PFA sections and in PBS+4% FBS+0.1% Triton X-100 for 1 h at room temperature.

In vitro cell culture staining for pSMAD2 and pSMAD5 staining in cytospun PDX cells, and MDA-MB-231 used a different protocol listed below.

Cells grown on glass coverslips, were fixed with ice cold methanol for 5 min at −20 C and washed with PBS 3× to remove methanol. Fixed cells were incubated in 3% BSA/PBS for 30 minutes at room temperature. Primary antibody incubation was performed by inverting the coverslip onto 30-35 ul of the antibody diluted 1:100 in 1% BSA/PBS for 60 minutes in a wet chamber at room temperature. This was followed by 3× washes with PBS. Secondary antibody incubation was performed by inverting the coverslip onto 30-35 ul of the antibody diluted 1:400 in 1% BSA/PBS for 60 minutes in a wet chamber at room temperature. This was followed by 3× washes with PBS.

All immunofluorescence sections and cells were mounted in ProLong Gold with DAPI reagent (Invitrogen) and images were taken with a Leica DMI4000 microscope or a Zeiss LSM710 Confocal Microscope.

Preparation of single cell suspension and flow cytometry. Mouse mammary cell subpopulations were obtained from 8-12 weeks-old virgin female C57BL/6 or pCx-GFP mice or mammary outgrowths arising from donor infected cells or NSG mice in which engineered 3T3-L1 cells were injected. These tissues were dissected and analyzed as described previously with modifications (Stingl et al., 2006). In brief, the glands were mechanically dissociated and digested in Advanced/DMEM media with collagenase/hyaluronidase and 100 Kunitz units of DNase I (Sigma-Aldrich) for 1 hr and 30 min for mouse breast tissue and overnight for human samples. The solution was neutralized with staining media (HBSS+2% bovine calf serum) followed by brief ACK lysis, trypsin and DNase/dispase treatments. Cells were filtered through a 40-μm mesh, counted and resuspended in 10⁷ cells/ml in staining media. For FACS analysis and sorting, cells were blocked for 10 min with 1 μg/ml rat IgG. Cells were washed and stained with antibodies at dilutions determined by titration experiments. The cocktail of antibodies used is described in Table 2.

For human samples, informed consent was obtained and normal mammary glands or breast tumor specimens were collected according to guidelines from Stanford University's Institutional Review Board. When cells were isolated from Patient-Derived Xenograft (PDX) tumor samples, Applicants used anti-H2Kd-Pacific Blue antibody to remove the mouse cells. Live/dead discrimination was obtained with DAPI (4′,6-diamidino-2-phenylindole). Analyses and sorting were performed using a FACSAria II (BD Biosciences). Flow cytometry was performed with a 100 or 130 μM nozzle on a BD FACSAria II using FACSDiva software. Debris and doublets were excluded by sequential gating. The cells were double sorted and those samples that had less than 80-90% purity were discarded.

For PDX infection purposes, breast tumor cells were stained with biotin anti-mouse H-2Kd microbeads, passed through a column and the negative fraction was recollected (Miltenyl BioTec).

Injection of engineered L1 cells, mammary cell transplantation assay and readout. Engineered 3T3-L1 cells were irradiated and 2.5 10e6 cells were injected in two locations of the adult NSG mammary fat pat. In other experiments, mammary cells were injected into cleared fat-pad of syngeneic weaning age mice. The total injection volume was 10 ul of sterile staining media containing 30% Matrigel. Outgrowths were analyzed at least 8 weeks after transplantation under fluorescence microscope and all the glands were stained with Carmine Aluminum for whole mount analysis (Plante et al., 2011).

Analysis of mammary duct structure and branching. Analysis was performed such that reader was blind to the control and experimental conditions. To convert ductal structures to skeletons, ducts from microscopic (20×) image of tissue sections for each condition were outlined, filled, and the entire image was converted to a binary image. The image was then processed using the “Skeletonize” function and “Analyze Skeleton functions” in ImageJ. The final skeletonized image was analyzed by the “Analyze Skeleton” tool. This image tags all ROI and counts all junctions, triple points, slab voxels, branches and measures their average and maximum branch length. Each field of view contained several independent ductal elements. Thus, several ROI, or ductal elements, were obtained for each field of view. For each image, Applicants sorted all ROI obtained and only analyzed structures that contained three or more branches, in order to remove any potential artifacts. From these ROI, average length of all branches was obtained, and branch length multiplied by the number of branches gave an estimate of length of the ROI. To calculate the average fractional area around mammary ducts, Applicants outlined ROI (Regions of Interest) around mammary trees of at least three, low magnification, fields of view per mouse. Applicants then used the Image J “fill function” the ROI, calculated the area and divided this number by the total area of the image. Applicants then compared the average fractional area covered by mammary trees in adult mice in various experimental conditions.

RNA isolation and expression assays. Total RNA was isolated using TRIZOL reagent following manufacturer's instructions. Glycogen was used as a carrier (Invitrogen). Complementary DNA was obtained using SuperScript III First Strand Synthesis following the manufactures instructions (Invitrogen). For mRNA analysis of the sorted mouse cells, a pre-amplification step before the PCR was performed. For the real time PCR, TaqMan primers were used (Table 3) (Applied Biosystems). Gapdh or Actb were used to normalize the expression values. The abundance of each gene was measured by using the 7900HT Fast Real-Time PCR System (Applied Biosystems).

DNA isolation and copy number assays. The DNA from H2K-D and CD45 depleted cells obtained from the different PDXs was isolated using QIAmp DNA micro kit using manufacturer's instructions. For the analysis of the copy number the human TaqMan® Copy Number Assays (ThermoFisher) was used following the manufacture's guideline. Applicants used the RNase P as a copy number reference assay. The primers used are indicated in Table 3. The PCR was run using the 7900HT Fast Real-Time PCR System and the CopyCaller software v2.1 was used for the analysis.

Single cell gene expression measurements. Single cell gene expression experiments were done as previously described (Dichn et al., 2009; Flatz et al., 2011; Guo et al., 2010; Petriv et al., 2010). Briefly, Applicants used M96 qPCR Dynamic Array microfluidic chips (Fluidigm) with 96 genes and 96 sample inlets. Single cells were sorted by FACS into 96 well plates containing PCR mix (CellsDirect, Invitrogen) and RNase Inhibitor (SuperaseIn, Invitrogen). The mRNA from the cell lysates was reverse-transcribed (15 minutes at 50° C., 2 minutes at 95° C.) and pre-amplified for 20 PCR cycles (each cycle: 15 sec at 95° C., 4 minutes at 60° C.). The resulting amplified cDNA from each one of the cells was inserted into the chip sample inlets with Taqman qPCR mix (Applied Biosystems). Individual assays (i.e. gene-specific TaqMan primer/probe sets, listed as Auxiliary materials, were inserted into the chip assay inlets. The chip was loaded for one hour in a chip loader (Nanoflex, Fluidigm) in order to combinatorially mix every sample with every assay and to partition individual reactions. The chip was then transferred to a reader (Biomark, Fluidigm) for thermocycling (40 cycles) and fluorescent quantification. qPCR amplification curves were analyzed using custom software (Fluidigm) in order to calculate the qPCR threshold cycles.

Single cell gene expression data analysis. Single cell gene expression data was analyzed using Matlab® (version 7.3.0.267 (R2006b), Math Works). Genes which showed low quality qPCR curves, or which had no biological relevance were discarded. Cells for which the “housekeeping” genes Actb or Gapdh were not expressed were discarded. Prior to performing further analyses, Applicants performed quantile-normalization on the data (1970): for each gene independently the single cell qPCR threshold cycle values were substituted by their ranks (the smallest value was given the rank 1, the second smallest was given rank 2 etc.). All non-expressing cells (which were formally assigned an infinite threshold cycle by the qPCR machine) were given the maximal rank (which is equal to the number of cells in the sample and is the same for all genes). Hierarchical clustering of the normalized data was done using standard algorithms (Matlab command: clustergram) with correlation distance metric and complete linkage. Principal component analysis (PCA) on the quantile-normalized gene expression data was done using standard algorithms (Matlab command: princomp). The first and second principal components were plotted. Cells from different populations (MRU, MYO, MaCFC, CD24^{med}CD49f^{neg}, and Zeb1±/Krt17^{neg} MRU cells) were plotted with different colors. Zeb1⁺/Krt17^{neg} MRU cells were defined as MRU cells that expressed Zeb1 and did not express Krt17. All other populations were defined according to FACS sorting gates. All single cell qPCR threshold cycle data, both before and after normalization, can be found as part of Supplementary Information, organized in Excel® spreadsheets.

The boxplots and violin plots in FIG. 8 were generated in R v3.6.0 using gene expression data and cluster labels from the Marioni dataset of normal mouse breast development (Bach et al., 2017).

Luciferase reporter assay. Twenty thousand 3T3-L1 cells growing in 96-well plate were transfected with Lipofectamine 2000 (Invitrogen) according to the manufacturer's instructions. Applicants used 250 ng pGL3 BRE-luciferase plasmid kindly provided by Martine Roussel and Peter ten Dijike (Addgene plasmid #45126). Twenty nanograms of pRL-TK Renilla luciferase vector (Promega) was added to each reaction to monitor transfection efficiency. Media was changed to LEFTY1 and/or BMP7 containing media 16 hours previous collection of the transfected cells. Luciferase activities were measured 72 h after the transfection using the Dual-Luciferase Reporter Assay System (Promega) and the data was normalized to Renilla luciferase activity. All experiments were performed in duplicate with data pooled from three independent experiments.

Infection of primary breast mammary and PDX cells. Cells were transduced overnight in breast organoid media with 25 m.o.i. of the virus. Normal primary breast cells were infected in suspension in a tube and incubated at 5% CO2 and 37° C. at least for 16 h. Human tumor xenograft cells were spin-infected for 2 hr with a 50 m.o.i. followed by overnight incubation at 5% CO2 and 37° C. in breast organoid media. Applicants observed similar transduction efficiency between control and experimental virus.

Lentiviral vectors and preparation. For pEGFP-C3-Lefty1, Lefty 1 was PCR cloned but using EcoRV-NotI and XbaI as flacking restriction sites from the IMAGE: 5221120 plasmid clone. The Lefty 1 product was cloned into EcoRV and XbaI sites of pEGFP-C3 (discontinued product from Clontech). For HIV-Lefty1-ZsG, Lefty1 was subcloned from pEGFP-C3-Lefty1 using Not1 and XbaI into pEIZ-HIV-ZsGreen vector (kindly provided by Dr. Zena Werb) (Welm et al., 2008). For HIV-Bmp7-Che, Bmp7 was PCR cloned from Bmp7 cDNA clone (Origene; MC201085) using the primers described in Table 3. Primers had NotI and XbaI restriction sites flanking Bmp7 gene. The Bmp7 product was cloned into HIV-Che, which was produced as described from pEIZ-HIV-ZsGreen vector (Shimono et al., 2009).

To knock-down mouse Lefty1 and human LEFTY1 PSICO-R lentiviral vector was used (Ventura et al., 2004). Different shRNAs were designed and cloned into pSICO-R as described. The sequences of the shRNAs cloned are described in Table 3. The efficiency of knock-down was tested by transfecting HEK293T cells with pSICO-R-shLefty1 constructs with pEGFP-C3-Lefty 1 at 1:4 and 1:20 ratio. Decrease on GFP signal was used to calculate the percentage of inhibition of the shLefty sequences.

Lentiviruses were produced and tittered as described (Tiscornia et al., 2006) but Applicants used Lipofectamine 2000 as transfection method and a second-generation lentivirus system. Supernatants were harvested 72 hr post-transfection; viral particles were concentrated by ultra-centrifugation and viral titers (transduction units) were calculated by FACS analyses taking into consideration the percentage of HEK293T GFPPS or CherryPos labelled cells. Only viral preparations higher than 109 viral particles/ml were used for Applicants' primary mammary cells' infection experiments.

DUOLink Proximity Ligation Amplification (PLA) assay. For the PLA assay (DUOLink, OLink Biosciences, Sigma-Aldrich #DUO92102), fibroblasts were seeded on 13 mm glass coverslips previously coated with Poly-L-Lysine (Millipore). The cells were fixed with ice cold 100% Methanol for 5 minutes at −20° C. and then rehydrated thrice in PBS for 5 min each. Coverslips were blocked for 30 min with PBS+3% BSA and then incubated with appropriate dilution of primary antibodies in PBS+1% BSA for 1 h in a moist environment at room temperature. Rabbit anti-Lefty1 and Thermo mouse anti-BMPR2 were used to characterize the interaction between Lefty and BMPR2 (Table 2). As a positive control, rabbit anti-BMP7 and mouse anti-BMPR2 were used to validate the interaction between BMP7 and BMPR2 (Table 2). As a negative control, rabbit and mouse Anti-IgGs (Millipore) were used in 1:200 dilutions. Subsequently, manufacturer's instructions were followed to complete the PLA assay.

Co-immunoprecipitation assay. Co-Immunoprecipitation experiments were carried out using the Pierce CO-IP Kit (#26149, Thermo Fisher Scientific) as per the manufacturer's protocol. For LEFTY-1 and BMPR2 interaction, fibroblasts were grown in complete medium in a 15 cm dish to reach 90% confluence and treated with LEFTY-1 overnight (200 ng/ml; R&D Systems). These were lysed with 1.5 mL ice cold IP Lysis Buffer containing protease and phosphatase inhibitors (Halt Protease inhibitor cocktail; ThermoFisher), for 1 h at 4° C. upon gentle agitation. For the antibody immobilization step, 20 μg of rabbit anti-Lefty or 20 μg of rabbit anti-BMPR2 (Table 2), or as a control 20 μg rabbit IgG, were diluted onto the AminoLink Plus Coupling Resin. The cell lysates were precleared with control agarose resin and co-immunoprecipitation was carried out by adding 1 mg of the precleared cell lysate to the antibody immobilized resin, with end over end mixing at 4° C. overnight. After elution into 50 μL, the sample was analyzed by SDS-PAGE gel and followed by immunoblotting to detect protein-protein interaction.

Western blotting. Fifty microliters of IP elute was loaded on a 4-20% gradient SDS-PAGE gel using appropriate sample buffer, and run at 80 volts for 90 min. SDS-PAGE gels were transferred onto PVDF membranes for 120 min at 70 v (#IPFL00010, Millipore, Billerica, MA). Membranes were blocked with 5% BSA-TBS and subsequently probed with primary antibodies diluted in TBS+5% BSA+0.05% Tween-20, overnight at 4° C. Primary antibodies against LEFTY1 and BMPR2 used are described in Table 2. Incubation with secondary antibodies containing fluorophores at 1:10,000 dilution (IRDye 800CW conjugated anti-rabbit #926-32211, LI-COR Biosciences, Lincoln, NE) enabled visualization on the Odyssey Infrared Imaging System from LI-COR Biosciences.

Quantification and statistical analysis. Graphs are represented as bars showing average±S.D./S.E.M. error bars. For stem cell frequency analysis, ELDA software was used. Variance was analyzed using the F-test P values of different statistical tests and their adjustments were calculated using GraphPad Prism software. For animal studies, sample size was not predetermined to ensure adequate power to detect a pre-specified effect size, no animals were excluded from analyses, experiments were not randomized and investigators were not blinded to group allocation during * refers to P value<0.05; ** refers to P value<0.01; *** refers to P value<0.001; refers to P value<0.0001.

TABLE 1
Description of patient-derived xenograft (PDX) models.

| | | | | Treatment | Type of | Gender/Age/ | Recurrent |
|---|---|---|---|---|---|---|---|
| Tumor | Hormone status | Type | Grade | (Yes/No) | treatment | Ethnicity | cancer? * |
| PDX #1 | ER^{neg}PR^{neg}HER^{neg} | Invasive ductal | III | No | | F/61 y/Caucasian | No/Yes |
| | | carcinoma | | | | | |
| PDX #2 | ER^{neg}PR^{neg}HER^{neg} | Invasive ductal | III | Yes | Chemotherapy** | F/54 y/Caucasian | **Yes/Yes |
| | | carcinoma | | | | | (metastasis) |
| PDX #3 | ER^{neg}PR^{neg}HER^{neg} | Invasive ductal | III | Yes | Chemotherapy | F/46 y/African | No/Yes |
| | | carcinoma | | | | American | |
| PDX #4 | ER^{pos}PR^{pos}HER^{neg} | Invasive ductal | III | Yes | Chemotherapy | F/31 y/Caucasian | Yes/Yes |
| | | carcinoma | | | | | (local and |
| | | | | | | | metastasis) |
| PDX #5 | ER^{neg}PR^{neg}HER^{neg} | Invasive ductal | III | Yes | Chemotherapy | F/51 y/African | Yes/Yes |
| | | carcinoma | | | | American | |

* Recurrent cancer before sample was collected/after collection of the sample.
**Prior acute promyelocytic leukemia.
Abbreviations: ER: estrogen; PR; progesterone; HER2: human epidermal growth factor receptor 2; pos: positive; neg; negative; F; female; y: years old.

TABLE 2
Primary antibody dilutions.

| | Specie of | | | |
|---|---|---|---|---|
| Antibody | the samples | Application | Dilution | Clone |
| H2Kd-biot | Xenograft | FC | 1:50; | SF-1.1 |
| or -PacB | | | 1:100 | |
| CD45 microbeads | Xenograft | FC | 1:10 | |
| CD31-PacB | Mouse | FC | 1:200 | 390 |
| CD45-PacB | Mouse | FC | 1:200 | 30-F11 |
| Ter119-PacB | Mouse | FC | 1:200 | TER-119 |
| CD49f-PE/Cy5 | Mouse/ | FC | 1:40 | GoH3 |
| | Human | | | |
| CD24-PE | Mouse | FC | 1:400 | 30-F1 |
| EpCAM | Human | FC | 1:100 | 9C4 |
| CD66a | Mouse | FC | 1:100 | CC1 |
| CD61 | Mouse | FC | 1:100 | 2C9.G2 |
| Thy 1.2 | Mouse | FC | 1:200 | 53-2.1 |
| Krt8 | Mouse | IF | 1:200 | TROMA-I |
| Krt14 | Mouse | IF | 1:200 | PRB-155P |
| | Mouse | IF | 1:100 | ab192694 |
| Sma | Mouse | IF | 1:100 | ab112022 |
| | | | | (discontinued) |
| | Mouse | IF | 1:200 | ab21027 |
| Lefty1 | Mouse | IF | 1:200 | ab22569 |
| | Mouse | Co-IP | 20 μg | |
| | Mouse | PLA | 1:200 | |
| | Mouse | WB | 1:1000 | |
| Bmp7 | Mouse | IF | 1:500 | ab27569 |
| | Mouse | PLA | 1:200 | ab56023 |
| Bmpr2 | Mouse | Co-IP | 20 μg | ab96826 |
| | Mouse | PLA | 1:200 | MA5-15827 |
| | Mouse | WB | 1:1000 | CST6979S |
| p-smad2 | Mouse | IF | 1:100 | ab53100 |
| p-smad5 | Mouse | IF | 1:400 | ab92698 |
| Nodal | Mouse | IF | 1:200 | ab55676 |

Abbreviations: FC: flow cytometry; IF: Immunoflourescence; IP: immunoprecipitation; WB: western blot; PLA: Proximity ligation amplification assay; Krt: cytokeratin; SMA: smooth muscle actin; PacB: Pacific blue; PE: Phycoerythrin.

TABLE 3
Sequence of the primers used in the Example.

| Name primers | Sequence (5′-3′) or Reference | Application |
|---|---|---|
| HIV-Bmp7 s | ATGTGCGGCCGCACCATGCACGTGCGCTCGCTGCGCGCTG | HIV cloning |
| | (SEQ ID NO: 16) | |
| HIV-Bmp7 as | GC TCTAGA CTA GTG GCA GCC ACA GGC CCG GAC CAC | HIV cloning |
| | (SEQ ID NO: 17) | |
| pEGFP-C3 | CAGAATTCGCGGGCCGCACCATGCCATTCCTGTGGCTCTG | HIV cloning |
| Lefty1 s | (SEQ ID NO: 18) | |
| pEGFP-C3 | CTTCTAGACTATGGCTGCAGCCTCCTGG (SEQ ID NO: 19) | HIV cloning |
| Lefty1 as | | |
| shLefty1 #1 s | TGGACAAGGCTGATGTGGAATTCAAGAGATTCCACATCAGCCTTGTC | HIV cloning |
| | CTTTTTTC (SEQ ID NO: 20) | |
| shLefty1 #1 as | TCGAGAAAAAAGGACAAGGCTGATGTGGAATCTCTTGAATTCCACAT | HIV cloning |
| | CAGCCTTGTCCA (SEQ ID NO: 21) | |
| shLefty1 #2 s | TGCAGGTTCCTGGTGTCAGATTCAAGAGATCTGACACCAGGAACCTG | HIV cloning |
| | CTTTTTTC (SEQ ID NO: 22) | |
| shLefty1 #2 as | TCGAGAAAAAAGCAGGTTCCTGGTGTCAGATCTCTTGAATCTGACAC | HIV cloning |
| | CAGGAACCTGCA (SEQ ID NO: 23) | |
| shLEFTY1 s | TCGAACTGCTGATGGACAAATGTTCAAGAGACATTTGTCCATCAGCA | HIV cloning |
| | GTTCATTTTTTC (SEQ ID NO: 24) | |
| shLEFTY1 as | TCGAGAAAAAATGAACTGCTGATGGACAAATGTCTCTTGAACATTTG | HIV cloning |
| | TCCATCAGCAGTTCAA (SEQ ID NO: 25) | |
| Gapdh | Mm99999915_g1 | Multiplex PCR |
| Lefty1 | Mm00438615_m1 | Multiplex PCR |
| Krt8 | Mm04209403_g1 | Multiplex PCR |
| Krt14 | Mm00516870_mH | Multiplex PCR |
| Acvr1 | Mm01331069_m1 | Multiplex PCR |
| Acvr1b | Mm00475712_m1; Mm00475713_m1 | Multiplex PCR |
| Acvr1c | Mm03023957_m1 | Multiplex PCR |
| Acvr2a | Mm00475713_m1; Mm01331095_m1 | Multiplex PCR |
| Acvr2b | Mm00431667_g1; Mm01348450_g1; Mm00431664_m1 | Multiplex PCR |
| Bmpr1a | Mm00477650_m1 | Multiplex PCR |
| Bmpr1b | Mm03023971_m1 | Multiplex PCR |
| Bmpr2 | Mm00432134_m1 | Multiplex PCR |
| LEFTY1 | Hs01016456_mH | Multiplex PCR |
| LEFTY1 | Hs00812324_cn | Copy number PCR |
| RNase P | Ref# 4403326 | Copy number PCR |

KEY RESOURCES TABLE

| | | |
|---|---|---|
| REAGENT or RESOURCE | SOURCE | IDENTIFIER |

Antibodies

| | | |
|---|---|---|
| H2Kd-biot or -PacB | BD; BioLegend | SF-1.1 |
| CD45 microbeads | Myltenyi | |
| CD31-PacB | BioLegend | 390 |
| CD45-PacB | BioLegend | 30-F11 |
| Ter119-PacB | BioLegend | TER-119 |
| CD49f-PE/Cy5 | BD | GoH3 |
| CD24-PE | eBioscience | 30-F1 |
| Krt8 | DSHB | TROMA-I |
| Krt14 | Covance | PRB-155P |
| Krt14 | Abcam | ab192694 |
| Sma | Abcam | ab112022 |
| | | (discontinued) |
| Sma | Abcam | ab21027 |
| Lefty1 | Abcam | ab22569 |
| Bmp7 | Abcam | ab27569 |
| Bmp7 | Abcam | ab56023 |
| Bmpr2 | Abcam | ab96826 |
| Bmpr2 | ThermoFisher | MA5-15827 |
| Bmpr2 | Cell Signaling | CST6979S |
| p-smad2 | Abcam | ab53100 |
| p-smad5 | Abcam | ab92698 |
| Total smad2 | BD Biosciences | 562586 |
| Total smad5 | Cell Signaling | CST12534S |
| Nodal | Abcam | ab55676 |
| CD66 | eBioscience | 17-0661-80 |
| CD61 | Biolegend | 104311 |
| Thy-1 | eBioscience | 25-0902-82 |
| EpCAM | Biolegend | 324222 |
| β-Actin | Cell Signaling | 3700S |
| Ki67 | Abcam | ab15580 |

Biological Samples

Patient derived xenografts are listed
in Table 1

Chemicals, Peptides, and Recombinant Proteins

| | | |
|---|---|---|
| Y-27632 | R&D Systems | |
| R-spondin3 | R&D Systems | |
| EGF | R&D Systems | |
| Noggin | R&D Systems | |
| Growth Factor Reduced Matrigel | Becton Dickinson | |
| LEFTY-1 | R&D Systems | 994-LF |
| BMP7 | R&D Systems | 5666-BP |
| BMP2 | R&D Systems | 355-BM |
| BMP4 | R&D Systems | 314-BP |
| CRIPTO-1 | R&D Systems | 1538-CR |
| NODAL | R&D Systems | 1315-ND |
| DAPI | Invitrogen | |
| Collagenase/Hyaluronidase | Sigma | |
| DNase I | Sigma | |
| Lefty Blocking Peptide | Santa Cruz | sc-365845 |
| LDN-193189 | Selleckchem | Catalog No.S2618 |

Critical Commercial Assays

| | | |
|---|---|---|
| Dual-Luciferase Reporter Assay System | Promega | |
| DUOLink | Sigma | DUO92102 |
| Pierce CO-IP Kit | ThermoFisher | |

Experimental Models: Cell Lines

| | |
|---|---|
| HEK293 | ATCC |
| MDA-MD-157 | ATCC |
| 3T3-L1 | ATCC |
| MDA-MB-231 | ATCC |
| COMMA-D | ATCC |

Experimental Models: Organisms/Strains

| | |
|---|---|
| C57BL/6 mice | Jackson Labs |
| NOD scid gamma (NSG) mice | Jackson Labs |
| pCx-GFP | Kind gift of Dr. |
| | Irving Weissman |

Oligonucleotides

| | | |
|---|---|---|
| HIV-Bmp7 s | ATGTGCGGCCGC | This Example |
| | ACCATGCACGTG | |
| | CGCTCGCTGCGC | |
| | GCTG (SEQ ID NO: | |
| | 16) | |
| HIV-Bmp7 as | GC TCTAGA CTA | This Example |
| | GTG GCA GCC | |
| | ACA GGC CCG | |
| | GAC CAC (SEQ ID | |
| | NO: 17) | |
| pEGFP-C3 Lefty1 s | CAGAATTCGCGG | This Example |
| | GCCGCACCATGC | |
| | CATTCCTGTGGC | |
| | TCTG (SEQ ID NO: | |
| | 18) | |
| pEGFP-C3 Lefty1 as | CTTCTAGACTAT | This Example |
| | GGCTGCAGCCTC | |
| | CTGG (SEQ ID NO: | |
| | 19) | |
| shLefty1 #1 s | TGGACAAGGCTG | This Example |
| | ATGTGGAATTCA | |
| | AGAGATTCCACA | |
| | TCAGCCTTGTCC | |
| | TTTTTTC (SEQ ID | |
| | NO: 20) | |
| shLefty1 #1 as | TCGAGAAAAAA | This Example |
| | GGACAAGGCTGA | |
| | TGTGGAATCTCT | |
| | TGAATTCCACAT | |
| | CAGCCTTGTCCA | |
| | (SEQ ID NO: 21) | |
| shLefty1 #2 s | TGCAGGTTCCTG | This Example |
| | GTGTCAGATTCA | |
| | AGAGATCTGACA | |
| | CCAGGAACCTGC | |
| | TTTTTTC (SEQ ID | |
| | NO: 22) | |
| shLefty1 #2 as | TCGAGAAAAAA | This Example |
| | GCAGGTTCCTGG | |
| | TGTCAGATCTCT | |
| | TGAATCTGACAC | |
| | CAGGAACCTGCA | |
| | (SEQ ID NO: 23) | |
| shLEFTY1 s | TCGAACTGCTGA | This Example |
| | TGGACAAATGTT | |
| | CAAGAGACATTT | |
| | GTCCATCAGCAG | |
| | TTCATTTTTTC | |
| | (SEQ ID NO: 24) | |
| shLEFTY1 as | TCGAGAAAAAAT | This Example |
| | GAACTGCTGATG | |
| | GACAAATGTCTC | |
| | TTGAACATTTGT | |
| | CCATCAGCAGTT | |
| | CAA (SEQ ID NO: | |
| | 25) | |
| Gapdh | Mm99999915_g1 | ThermoFisher |
| Lefty1 | Mm00438615_m1 | ThermoFisher |
| Krt8 | Mm04209403_g1 | ThermoFisher |
| Krt14 | Mm00516870_mH | ThermoFisher |
| Acvr1 | Mm01331069_m1 | ThermoFisher |
| Acvr1b | Mm00475712_m1; | ThermoFisher |
| | Mm00475713_m1 | |
| Acvr1c | Mm03023957_m1 | ThermoFisher |
| Acvr2a | Mm00475713_m1; | ThermoFisher |
| | Mm01331095_m1 | |
| Acvr2b | Mm00431667_g1; | ThermoFisher |
| | Mm01348450_g1; | |
| | Mm00431664_m1 | |
| Bmpr1a | Mm00477650_m1 | ThermoFisher |
| Bmpr1b | Mm03023971_m1 | ThermoFisher |
| Bmpr2 | Mm00432134_m1 | ThermoFisher |
| LEFTY1 | Hs01016456_mH | ThermoFisher |
| LEFTY1 | Hs00812324_cn | ThermoFisher |
| RNase P | Ref# 4403326 | ThermoFisher |

Recombinant DNA

| | | |
|---|---|---|
| HIV-Lefty1-ZsG | | |
| HIV-Che | | |
| HIV-Bmp7-Che | | |
| pGL3 BRE-luciferase | Kind gift of Martine | Addgene |
| | Roussel and Peter | |
| | ten Dijike, #45126 | |
| pRL-TK Renilla luciferase vector | | Promega |
| pEGFP-C3 | | Clontech |
| pEIZ-HIV-ZsGreen | Kind gift flof Dr. | |
| | Zena Werb | |
| Bmp7 cDNA clone | MC201085 | Origene |
| PSICO-R | | Addgene |

Software and Algorithms

| | |
|---|---|
| ImageJ | |
| Prism | GraphPad |
| | Software |
| CopyCaller software v2.1 | |
| Matlab ® (version 7.3.0.267 (R2006b) | Math Works |
| R v3.6.0 | R Studio |
| Webtool | ELDA: extreme |
| (http://bioinf.wehi.edu.au/software/ | limiting dilution |
| elda/) | analysis |

### Example 2: LEFTY Proteins and Breast Cancer Cell Proliferation

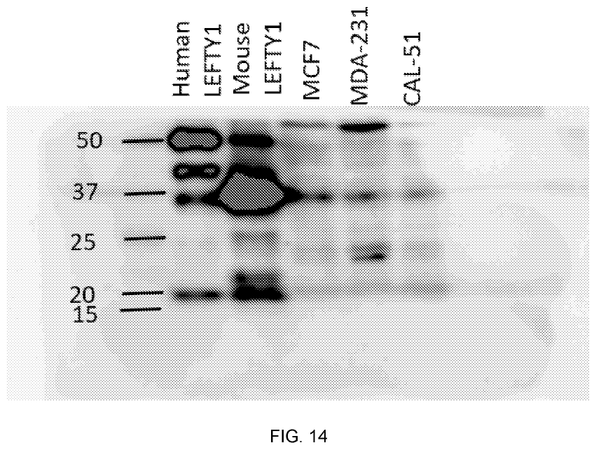

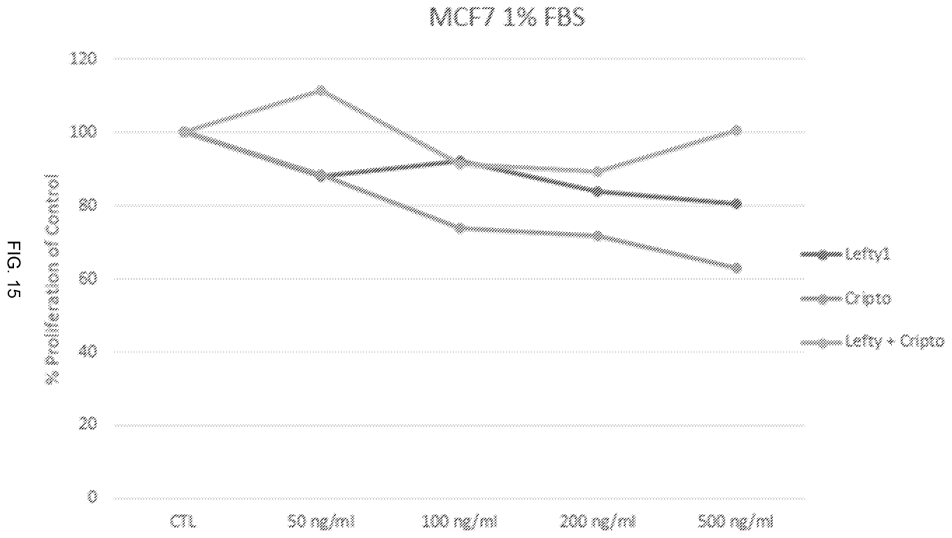

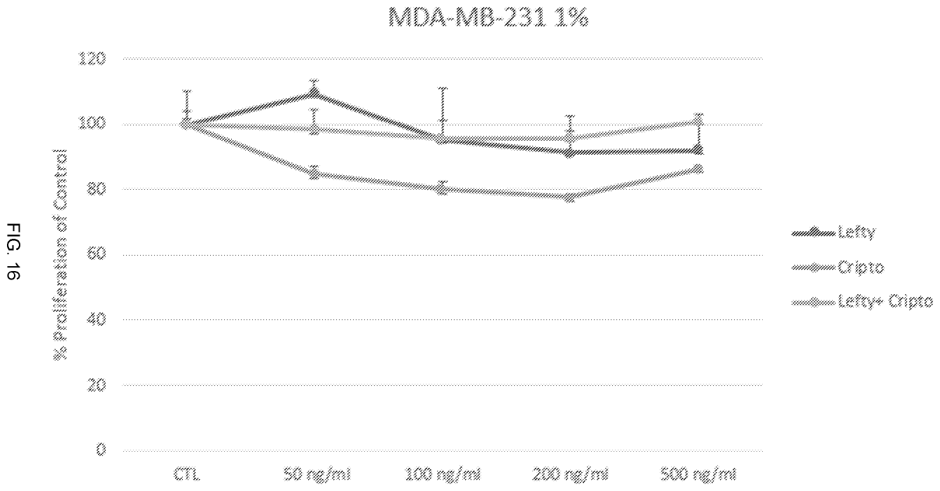

FIG. 14 show that the MCF7, MDA-231, and CA51 breast cancer cell lines express LEFTY1 protein. These data show that LEFTY1 is expressed by hormone receptor positive and triple negative breast cancer cells.

Dilute RIPA buffer with 10× Protease Inhibitors (and 10× Phosphatase Inhibitors if measuring phosphorylated proteins) in RIPA buffer (4° C.). Add RIPA buffer to cell pellet/plate well. Adjust the volume depending on the number of cells. Incubate 10′ at 4° C. Centrifugate the sample for 15′ at 14 000 G and keep the supernatant. Quantify protein by BCA method. PRECAST THE ACRYLAMIDE GEL. Adjust the volume of protein extract to achieve the same concentration of proteins in all the samples (the amount of total protein loaded in a gel will depend on the sample and the protein of interest; from 10-50 ug/well). Add 6× Laemmli Sample Buffer (RT). Boil the sample at 95° C. for 10 minutesConcentrate samples at 100V. Run at 130V until bromophenol blue front achieves the end of the gel. Blot the acrylamide gel in the PVDF membrane following Thermo Fisher iBlot2 equipment standard protocol. See the following video for detailed steps: https://www.youtube.com/watch?v=PN6ZMQWeMfI. Block the membrane by incubation of 5% w/v BSA in TBS-Tween 0.01% prepared from BSA powder (4° C.), 1 h at RT. Incubate overnight at 4° C. and movement with desired dilution of the primary antibody diluted in 1% BSA TBS-Tween 0.01%. Incubate during 1 h, at RT and movement with desired dilution of the primary antibody diluted in 1% BSA TBS-Tween 0.01%. Prepare 1:1 dilution of Reagents A and B of Pierce ECL Reagent Kit. Incubate the membrane with ECL for 1′. Visualize and acquire pictures with Syngene G Box. Incubate the membrane with 1:200 dilution of SCBT anti HSP70-HRP antibody.

FIG. 15 shows that Lefty1 and Cripto (Cripto1, TDGF1) synergize to increase the growth of MCF7 breast cancer cells. These data show that LEFTY1 and CRIPTO decrease the proliferation of hormone responsive breast cancer cells. However, the combination of both LEFTY1 and CRIPTO together increase the proliferation of hormone responsive breast cancer cells. This effect is dependent on the ratio of the two proteins, observed by the effect at 50 ng/ml and not at higher concentrations of the two proteins.

On Day 1, plate 1250 MCF7 cells in a volume of 50 ul/well of 1% FBS+DMEM (complete media) in each well of a 96-well microplate. On Day 2, prepare 2× dilutions of desired protein/inhibitors in complete media, with PBS as negative control (CTL) and add 50 ul to each well. Lefty (mouse Lefty 1 protein, R&D systems, catalog number 994-LF) and CRIPTO (human CRIPTO, R&D Sytems, catalog number 145-CR). On Day 8, calculate final volume of CyQuant (ThermoFisher, catalog number C35007) and add to each well according to manufacturer's instructions. Read fluorescence with plate reader with the excitation 485 nm and emission 530 nm.

FIG. 16 shows that Lefty1 and Cripto (Cripto1, TDGF1) synergize to increase the growth of MDA-MB-231 breast cancer cells. These data show that LEFTY1 and CRIPTO decrease the proliferation of triple negative breast cancer cells. However, the combination of both LEFTY1 and CRIPTO together increase the proliferation of hormone responsive breast cancer cells. This effect is dependent on the ratio of the two proteins, observed by the effect at 50 ng/ml and not at higher concentrations of the two proteins.

On Day 1, plate 1000 MDA-MB-231 cells in a volume of 50 ul/well of 1% FBS+DMEM (complete media) in each well of a 96-well microplate. On Day 2, prepare 2× dilutions of desired protein/inhibitors in complete media, with PBS as negative control (CTL) and add 50 ul to each well. Lefty (mouse Lefty1 protein, R&D systems, catalog number 994-LF) and CRIPTO (human CRIPTO, R&D Systems, catalog number 145-CR). On Day 8, calculate final volume of CyQuant (ThermoFisher, catalog number C35007) and add to each well according to manufacturer's instructions. Read fluorescence with plate reader with the excitation 485 nm and emission 530 nm.

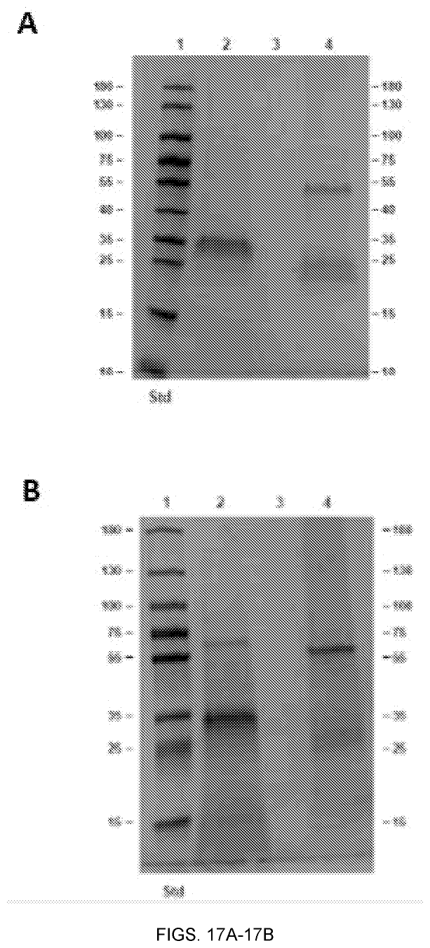

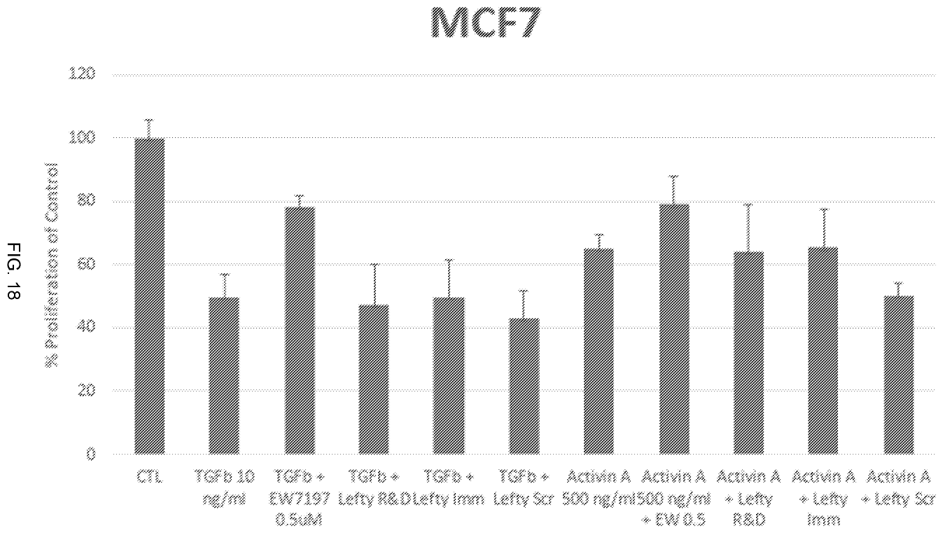

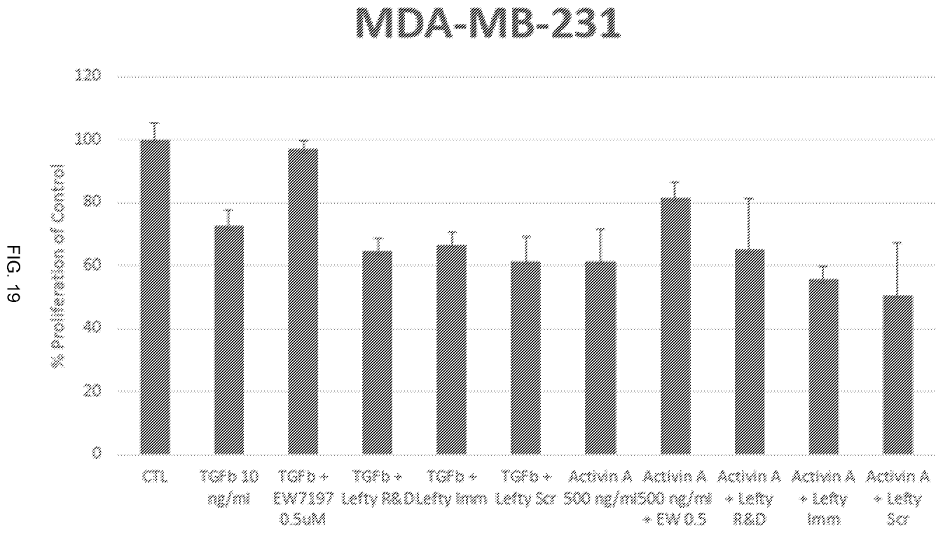

FIGS. 17A and 17B show SDS-Page analysis of 1189 IMM and 1189 SCR1 antigens. FIG. 17A shows 4-20% denaturing, reducing and non-reducing. SDS-PAGE analysis of 1189 IMM. Lane 1 refers to molecular weight marker PageRuler (Thermo Fisher) shown in kilodaltons; Lane 2 refers to 1189 IMM in reducing condition; Lane 3 refers to blank lane; Lane 4 refers to 1189 IMM in non-reducing condition. FIG. 17B shows 4-20% denaturing, reducing and non-reducing, SDS-PAGE analysis of 1189 SCR1. Lane 1 refers to molecular weight marker PageRuler (Thermo Fisher) shown in kilodaltons; Lane 2 refers to 1189 SCR1 in reducing condition; Lane 3 refers to blank lane; Lane 4 refers to 1189 SCR1 in non-reducing condition.

1189 IMM. 1189 SCR1 and 1189 SCR2 were expressed using a CHO based transient expression system and the resulting protein containing cell culture supernatants were clarified by centrifugation and filtration. 1189 IMM, 1189 SCR1 and 1189 SCR2 were purified using AKTA chromatography equipment, from cell culture supernatants via affinity chromatography. Purified proteins were dialyzed/buffer exchanged into phosphate buffered saline solution. The purity of protein was determined to be >95% as judged by reducing and denaturing Sodium Dodecyl Sulfate Polyacrylamide gels. Protein concentration was determined by measuring absorbance at 280 nm and calculated using the theorical extinction coefficient.

FIG. 18 shows TGFb1 (10 ng/ml) and Activin A (500 ng/ml) decrease MCF7 cell line proliferation, an effect that is rescued by the vactosertib (EW7197, an ALK5/ALK2/ALK4 inhibitor), but not with Lefty R&D (rhLefty2 protein, R&D Systems, cat no, Lefty Imm (human Lefty1-Rabbit Fc fusion protein), or Lefty Scr (human Lefty1-Human Fc fusion protein). These data show that human LEFTY1 and LEFTY2 proteins do not promote hormone responsive breast cancer cell proliferation by counteracting TGFB1 or Activin A signalling through ALK5, AL2 or ALK4.

On Day 1, plate 1250 MCF7 cells in a volume of 50 ul/well of 10% FBS+DMEM (complete media) in each well of a 96-well microplate. On Day 2, prepare 2× dilutions of desired protein/inhibitors in complete media, with PBS as negative control (CTL) and add 50 ul to each well. TGFB1 (10 ng/ml, R&D systems, catalog number 240-B), Lefty R&D (500 ng/ml, human Lefty2 protein, R&D systems, catalog number 746-LF-025), Activin A (500 ng/ml, human Activin A. Biolegend, catalog number 718502), EW7197 (0.5 uM, vactorsertib, Deltaclon, catalog number S7530). Lefty Imm (500 ng/ml, human Lefty1-Rabbit Fc fusion protein), Lefty Scr (500 ng/ml, human Lefty1-human Fc fusion protein) were custom generated. On Day 8, calculate final volume of CyQuant (ThermoFisher, catalog number C35007) and add to each well according to manufacturer's instructions. Read fluorescence with plate reader with the excitation 485 nm and emission 530 nm.

FIG. 19 shows TGFb1 (10 ng/ml) and Activin A (500 ng/ml) decrease MDA-MB-231 cell line proliferation, an effect that is rescued by the vactosertib (EW7197, an ALK5/ALK2/ALK4 inhibitor), but not with Lefty R&D (rhLefty2 protein, R&D Systems, cat no, Lefty Imm (human Lefty 1-Rabbit Fc fusion protein), or Lefty Scr (human Lefty1-Human Fc fusion protein). These data show that human LEFTY1 and LEFTY2 proteins do not promote triple negative breast cancer cell proliferation by counteracting TGFB1 or Activin A signalling through ALK5, AL2 or ALK4.

On Day 1, plate 1000 MCF7 cells in a volume of 50 ul/well of 10% FBS+DMEM (complete media) in each well of a 96-well microplate. On Day 2, prepare 2× dilutions of desired protein/inhibitors in complete media, with PBS as negative control (CTL) and add 50 ul to each well. TGFB1 (10 ng/ml, R&D systems, catalog number 240-B), Lefty R&D (500 ng/ml, human Lefty2 protein, R&D systems, catalog number 746-LF-025), Activin A (500 ng/ml, human Activin A. Biolegend, catalog number 718502), EW7197 (0.5 uM, vactorsertib, Deltaclon, catalog number S7530). Lefty Imm (500 ng/ml, human Lefty1-Rabbit Fc fusion protein), Lefty Scr (500 ng/ml, human Lefty1-human Fc fusion protein) were custom generated. On Day 8, calculate final volume of CyQuant (ThermoFisher, catalog number C35007) and add to each well according to manufacturer's instructions. Read fluorescence with plate reader with the excitation 485 nm and emission 530 nm.

### Example 3: Antibodies Against LEFTY Protein

The aim of this section of the project is to generate a panel of monoclonal antibodies against human LEFTY1 protein. (as LEFTY1 Protein shares high homology with human LEFTY2, Applicants can not exclude that the antibodies that will be generated are only against LEFTY1.

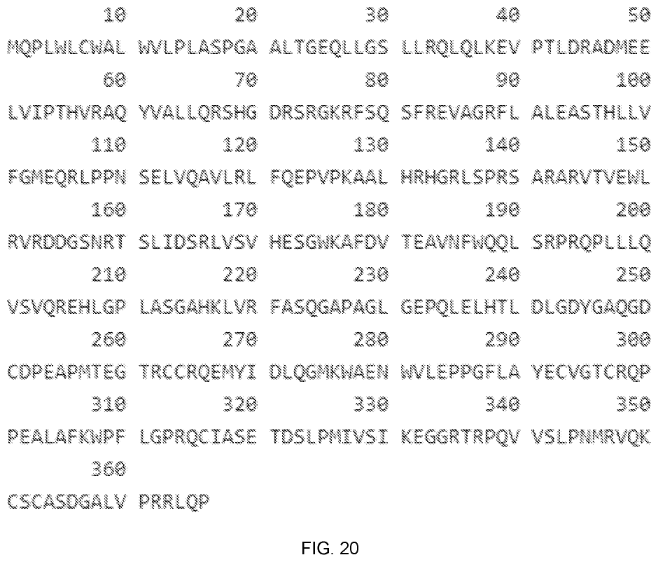

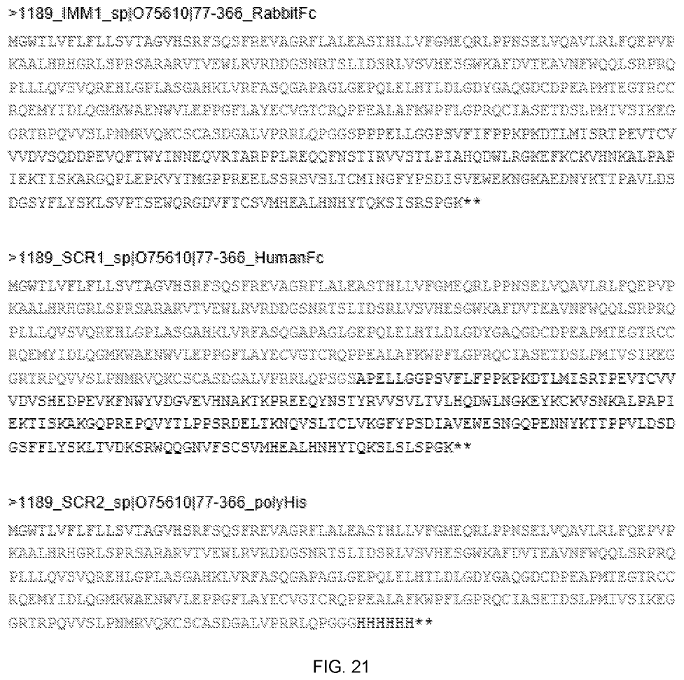

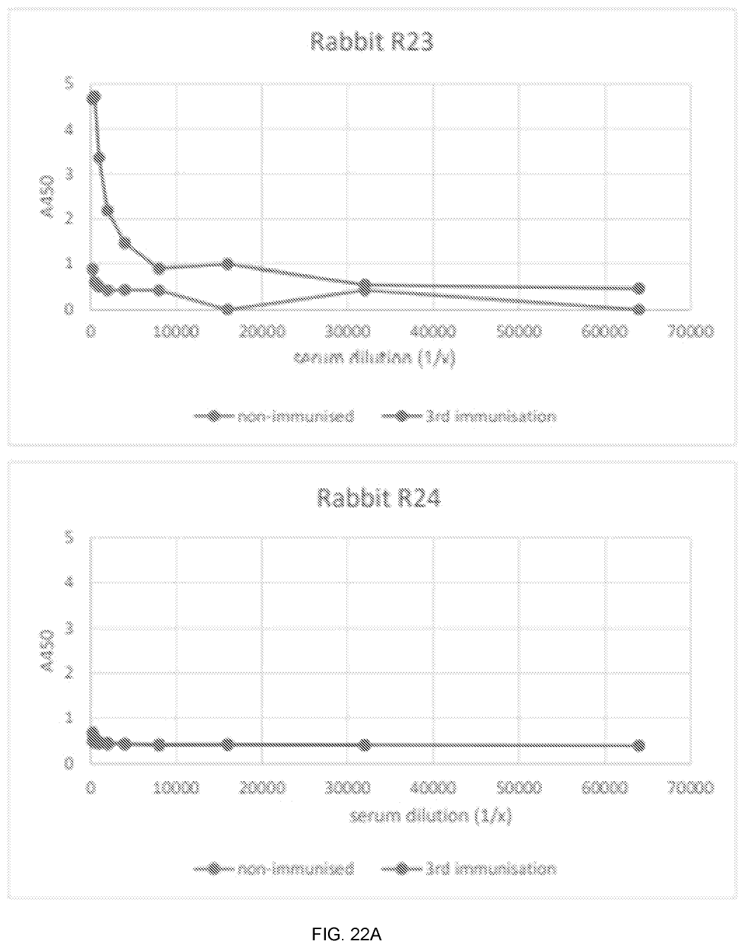

FIG. 20 shows the amino acid sequence of the human left-right determination factor 1 LEFTY1 (Homo Sapiens (human) sequence (UniProtKB-O75610 (LFTY1_HUMAN); gene ID: 10637), was used to generate 3D models of the protein of interest and design recombinant protein antigens.

FIG. 21 Sequences of the designed LEFTY1 human protein antigens. DNA coding for the amino acid sequence of 1189 IMM, 1189 SCR1 and 1189 SCR2 were synthesised and cloned into the mammalian transient expression plasmid pETE V2.

Fc protein fusions normally express well and are simple to purify with Protein A chromatography. The discrete domain formation also allows clean presentation of the target protein. In this approach, the mature protein sequence was expressed as part of a rabbit Fc fusion (1189 IMM). This construct was used to produce enough antigen protein in order to be injected into rabbits in order to generate monoclonal anti-LEFTY1 antibodies. The rabbit Fc fusion framework is immunogenically neutral in the host (rabbits), however the additional size of the protein stimulates the humoural response. Furthermore, Applicants generated two different screening constructs that were used to identify the antibody candidates generated in the rabbits. Applicants produced the same protein-LEFTY1-conjugated to a human Fc (1189 SCR1) and in addition a His-tagged variant, without the Fc (1189 SCR2).

FIGS. 22A and 22B depict results of immunization campaigns in rabbits after several rounds of 1189 IMM antigen injection.

Two rabbits (R23 and R24) were immunized three times with 1189 IMM in three-week intervals (FIG. 22A). Animals were boosted three weeks after the third immunization before spleen isolation. The blood sera were obtained Day 0 and 10 days after second and third immunization. Immune response was tested from blood sera using ELISA screening against LEFTY1 SCR1. The immune response to LEFTY1 SCR after the third immunisations measured by ELISA is shown in FIG. 22A. The titer of rabbit R23 was calculated as 1:4000. Rabbit R24 didn't generate a response. Rabbit R23 received two additional 1189 IMM injections. Immune response tested from rabbit R23 sera after the fifth immunization was significantly increased with ELISA titer 1:32000 (FIG. 22B). Rabbit R23 was boosted three weeks after the fifth immunization, animal was sacrificed three days after final boost, splenocytes were isolated and stored in liquid nitrogen until use.

FIG. 23 shows ELISA data, testing of 48 mIgG1-k library pools developed from rabbit R23, absorvance at 450 nm.

Splenocytes were harvested from rabbit R23 and panned on 1189-LEFTY1-SCR1 antigen for specific cell enrichment. RNA was isolated and cDNA was synthesized and used for VH (variable heavy) and VL (variable light) amplification and cloning into a mouse IgG1-k encoding two-cassette expression plasmid. Plasmid DNA from 48 antibody library pools was purified and transiently transfected into CHO cells for transient production of antibodies in serum-free media. Cell culture supernatants were tested for 1189-LEFTY1-SCR binding in ELISA. Applicants were able to identify 4 ELISA positive antibody libraries against LEFTY1 SCR (FIG. 23).

FIG. 24 shows an ELISA response of single clones isolated from library pools to LEFTY1.

Plasmid DNA from the four LEFTY1 specific ELISA positive pools was isolated and transfected into CHO cells for antibody transient production and subsequent analysis by ELISA on LEFTY1 SCR.

FIG. 25 shows a variable region sequence alignment of anti-LEFTY1 1189 antibody. Eight mIgG1-k ELISA positive clones to LEFTY1 were identified in the libraries of antibodies. The aminoacidic sequence of those clones were analyzed by sequencing. A unique anti-LEFTY1 antibody was identified. FIG. 25A shows variable region sequence alignment. Antibodies are clustered, identical or similar VH and VL are grouped, and CDR-s are marked with blue on consensus sequence (below). FIG. 25B shows CDR-s of anti-LEFTY antibodies isolated from rabbit Heavy chain CDR-s are designed H1, H2, H3 and light CDR-s L1, L2, L3, respectively. Distance between individual unique CDR regions are shown in the distance matrix.

FIG. 26 shows an ELISA response of anti-LEFTY1 1189specific mIgG1 antibody.

The mIgG1 antibody clone 1 H11 was transfected into CHO cells for transient production in 6-well format. Produced antibody supernatant was tested by ELISA on LEFTY1 SCR coated plates (FIG. 26). The ELISA titer of this antibody to its binding to LEFTY1 SCR antigen is about 16 ng/ml.

FIG. 27 shows Antibody heavy and light chain sequences. Heavy and light chain signal peptides and constant regions are underlines. Heavy and light chain variable regions are marked in bold.

FIG. 28 shows an anti-LEFTY1 antibody binds to human LEFTY1-Rabbit Fc fusion protein, human LEFTY1-human Fc fusion protein, and rhLEFTY1 (recombinant human LEFTY2) protein. This effect is reduced when a Lefty1 peptide with sequence PMIV SVKEGGRTRPQVVSLPNMRVQT (SEQ ID NO: 14) is added. These data show that anti-LEFTY antibody that targets the C terminal 326-357 amino acids of mouse Lefty 1 binds to human LEFTY1 fusion proteins and recombinant human LEFTY2.

Day 0, coat the ELISA plate (ThermoFisher Scientific MaxiSorp Surface (Plate wardrobe) with desired protein at 5 μg/ml dissolved in ELISA Coating buffer (0.2M Sodium Carbonate pH=9.4) (4° C.) of 50 μl/well. Incubate overnight at 4° C. Day 2, remove coating and wash plate 3 times for 5 minutes with Washing Buffer (0.05% Tween 20 in 1×PBS) at room temperature. BLOCKING: block the plate with 200 μl/well of Blocking Buffer (2% w/v of Bovine Scroalbumin (BSA) dissolved in 0.05% Tween 20/1×PBS) (4° C.) for 1 h at room temperature (RT). DETECTION ANTIBODY: remove blocking buffer and incubate the plate with 5 μg/ml of primary antibody (Santa Cruz Biotechnology, catalog number sc-36584) (50 μl/well) dissolved in 1% w/v BSA for 2 h at RT. Remove detection antibody and wash plate 3 times for 5 minutes with Washing Buffer (RT). SECONDARY ANTIBODY: incubate the plate with 1:5000 dilution of Secondary antibody diluted in 1% w/v BSA for 1 h at RT. Remove secondary antibody and wash plate 3 times for 5 minutes with Washing Buffer (RT). VISUALIZATION: incubate for 20 min at RT and darkness with 50 μl/well of 1:1 solution of TMB Substrate Reagent (4° C.) (BD Biosciences) according to manufacturer's instructions. STOP: Add 50 μl/well of ELISA Stop Buffer (2M H2SO4) (4° C.). MEASUREMENT: Measure absorbance at 450 nm with the Plate Reader.

FIG. 29 shows anti-Lefty1 antibodies and Lefty 1 peptide reduce the growth of MCF7 breast cancer cells. Lefty1 blocking peptide (LBP, derived from amino acids 327 to 352 at the C terminus of Lefty of mouse origin). Note that R&D system anti-Lefty1 antibody (which binds from Leu136 to Pro368, Accession #Q64280.1) does not affect the proliferation of MCF7 cells, showing that only certain portions of Lefty1 are important for MCF7 breast cancer cell growth. These data show that anti-LEFTY antibodies the bind to and peptides derived from specific portions of Lefty 1 protein sequence reduce the proliferation of hormone responsive breast cancer cells.

On Day 1, plate 1250 MCF7 cells in a volume of 50 ul/well of 10% FBS+DMEM (complete media) in each well of a 96-well microplate. On Day 2, prepare 2× dilutions of desired protein/inhibitors in complete media, with PBS as negative control (CTL) and add 50 ul to each well. R&D mAb (catalog number MAB994), SCBT mAb D6 (Santa Cruz Biotechnology, catalog number sc-365845), LBP (Santa Cruz Biotechnology, catalog number sc-365845 P). On Day 8, calculate final volume of CyQuant (ThermoFisher, catalog number C35007) and add to each well according to manufacturer's instructions. Read fluorescence with plate reader with the excitation 485 nm and emission 530 nm.

FIG. 30 shows anti-Lefty 1 antibodies and Lefty 1 peptide reduce the growth of MDA-MB-231 breast cancer cells. Lefty1 blocking peptide (LBP, derived from amino acids 327 to 352 at the C terminus of Lefty of mouse origin). Note that R&D system anti-Lefty1 antibody (which binds from Leu136 to Pro368, Accession #Q64280.1) does not affect the proliferation of MCF7 cells, showing that only certain portions of Lefty1 are important for MCF7 breast cancer cell growth. These data show that anti-LEFTY antibodies the bind to and peptides derived from specific portions of Lefty1 protein sequence reduce the proliferation of triple negative breast cancer cells.

On Day 1, plate 1000 MDA-MB-231 cells in a volume of 50 ul/well of 10% FBS+DMEM (complete media) in each well of a 96-well microplate. On Day 2, prepare 2× dilutions of desired protein/inhibitors in complete media, with PBS as negative control (CTL) and add 50 ul to each well. R&D mAb (catalog number MAB994), SCBT mAb D6 (Santa Cruz Biotechnology, catalog number sc-365845), LBP (Santa Cruz Biotechnology, catalog number sc-365845 P). On Day 8, calculate final volume of CyQuant (ThermoFisher, catalog number C35007) and add to each well according to manufacturer's instructions. Read fluorescence with plate reader with the excitation 485 nm and emission 530 nm.

FIG. 31 shows anti-LEFTY1 1189 monoclonal Ab reduces the proliferation of MCF7, MDA-MB-231 and CAL51 breast cancer cells. These data show that anti-LEFTY monoclonal antibody reduces the proliferation of hormone responsive and triple negative breast cancer cells.

On Day 1, plate 1000 MCF7, 1250 MDA-MB-231, or 1500 CAL51 cells in a volume of 50 ul/well of 10% FBS+DMEM (complete media) in each well of a 96-well microplate. On Day 2, prepare 2× dilutions of desired protein/inhibitors in complete media, with PBS as negative control (CTL) and add 50 ul to each well. 1186 full length anti-LEFTY monoclonal antibody was custom generated from a rabbit immunization campaign. On Day 8, calculate final volume of CyQuant (ThermoFisher, catalog number C35007) and add to each well according to manufacturer's instructions. Read fluorescence with plate reader with the excitation 485 nm and emission 530 nm.

FIG. 32 shows anti-LEFTY antibodies have different abilities to reduce the proliferation of breast cancer cells. C-terminal oriented antibodies reduce the proliferation of breast cancer cells that are dependent on LEFTY1 for their growth. The schematic represents the structure of LEFTY proteins in 2D, with annotations for the naturally occurring signal, propeptide, mature chain and proteolytic processing cleavage sites. The summary of the effect of different anti-LEFTY antibodies on the growth of MDA-MB-231 breast cancer cells are summarized, as well the region of LEFTY1 protein the are raised against. R&D Ab (R&D Systems, MAB994), Abcam Ab (Abcam, catalog number ab22569), SCBT Ab (Santa Cruz Biotechnology, catalog number sc-36584), HPA Ab (Human Protein Atlas, catalog number HPA056210).

FIG. 33 shows human LEFTY1 is copy number amplified in human cancers, making these cancers target indications for an anti LEFTY1 antibody treatment. The TCGA (The Cancer Genome Atlas, https://portal.gdc.cancer.gov/) database was searched for cancer that have either a gain of copy number (in red) or loss of copy number (blue) across datasets of different human cancers. Abbreviations: CNV (copy number variation, TCGA, The Cancer Genome Atlas; OV, Ovarian serous cystadenocarcinoma; SARC, sarcoma; BRCA.; Breast invasive carcinoma DLBC, Lymphoid Neoplasm Diffuse Large B-cell Lymphoma; ESCA, Esophageal carcinoma; UCS, Uterine Carcinosarcoma; SKCM, Skin Cutaneous Melanoma; LIHC, Liver hepatocellular carcinoma; LUAD, Lung adenocarcinoma; BLCA, Bladder Urothelial Carcinoma; STAD, Stomach adenocarcinoma; LUSC, Lung squamous cell carcinoma; CHOL, Cholangiocarcinoma; UCEC, Uterine Corpus Endometrial Carcinoma; PRAD, Prostate adenocarcinoma; CESC, Cervical squamous cell carcinoma and endocervical adenocarcinoma; READ, Rectum adenocarcinoma; GBM, Glioblastoma multiforme; UVM, Uveal Melanoma, MESO, Mesothelioma.

### Example 4: Humanized Antibodies Against LEFTY Protein

The sequence of the SCBT anti-lefty antibody was de novo sequenced; variable regions in black, mouse constant regions (kappa or gamma 1) are italic underlined.

>SCBT (light)
(SEQ ID NO: 26)
DILMTQAAPSISVTPGESVSISCRSSESLLHSNGNTYLYWFLQRPGQSP
QLLIYRKSNLASGVPDRFSGSGSGTAFTLRISRVEAEDVGVYYCMQQLE
YPLTFGGGTKLELKRADAAPTVSIFPPSSEQLTSGGASVVCFLNNFYPK
DINVKWKIDGSERQNGVLNSWTDQDSKDSTYSMSSTLTLTKDEYERHNS
YTCEATHKTSTSPIVKSFNRNEC
>SCBT (Heavy)
(SEQ ID NO: 27)
QVQLQQSGAELVRPGASVKLSCKALGYTFADYEMHWVRQTPVHGLEWTG
SIHPGSGGTAYDQRFKGKATLTADKSSSTAYMELSSLTSEDSAVYYCTF
YDLDYWGQGTTLTVSSAKTTPPSVYPLAPGSAAQTNSMVTLGCLVKGYF
PEPVTVTWNSGSLSSGVHTFPAVLQSDLYTLSSSVTVPSSTWPSETVTC
NVAHPASSTKVDKKIVPRDCGCKPCICTVPEVSSVFIFPPKPKDVLTIT
LTPKVTCVVVDISKDDPEVQFSWFVDDVEVHTAQTQPREEQFNSTFRSV
SELPIMHQDWLNGKEFKCRVNSAAFPAPIEKTISKTKGRPKAPQVYTIP
PPKEQMAKDKVSLTCMITDFFPEDITVEWQWNGQPAENYKNTQPIMDTD
GSYFVYSKLNVQKSNWEAGNTFTCSVLHEGLHNHHTEKSLSHSPGK

>OMED-001 (light)
(SEQ ID NO: 28)
DILMTQAAPSISVTPGESVSISCRSSESLLHSNGNTYLYWFLQRPGQSP
QLLIYRKSNLASGVPDRFSGSGSGTAFTLRISRVEAEDVGVYYCMQQLE
YPLTFGGGTKLELKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
>OMED-001 (heavy)
(SEQ ID NO: 29)
QVQLQQSGAELVRPGASVKLSCKALGYTFADYEMHWVRQTPVHGLEWTG
SIHPGSGGTAYDQRFKGKATLTADKSSSTAYMELSSLTSEDSAVYYCTF
YDLDYWGQGTTLTVSSASTKGPSVFPLAPSSKSTSGGTAALGCLVKDYF
PEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLS
PGK

FIG. 34 depicts structures of the SCBT antibody, OMED-001 and humanized candidates. The SCBT antibody depicts mouse kappa and mouse IgG1 regions. OMED-001 depicts mouse variable regions, human kappa and human IgG1 regions. Humanized candidates depict mouse CDRs grafted on human germlines of closest homology regions, human kappa and human IgG1 regions.

FIG. 35 presents the sequence of humanized candidates: 6 heavy chains and 4 light chains, which were done by CDR grafting into IGHV1-2 and IGKV2D-29 respectively followed by back mutations in residues within the vernier zone.

FIG. 36 presents the binding of OMED-001 to immobilized Lefty protein analyzed by ELISA. Purified OMED-001 or isotype control antibody at 100 ng/ml was incubated with immobilized lefty followed by colorimetric detection.

### Example 5: Lefty Effect Rescue on Mv1Lu Cell by OMED-001

To test antibody neutralization capacity in vitro, a well known Lefty sensitive assay was employed. The viability of Mv1Lu cells, originate in mink lungs, dose dependently decrease when they are exposed to Lefty1.

Briefly, a dose response assay with increasing amounts of Lefty was developed to establish the IC50 of the assay. Once determined Lefty IC50 value was 1.5 ugr/ml), cells were treated with Lefty alone and in combination with increasing concentrations of OMED-001 antibody in a 96 well plate. After 24 h of exposure, cell viability was assessed. FIG. 37 depicts Lefty 1 rescue by OMED-001.

### Example 6 OMED-001 Antibody rmLefty1 Neutralization on BMP-4 Inhibition

pGL3 BRE Luciferase plasmid is a well known expression plasmids that contains BMP responsive elements that trigger luciferase expression when activated by BMP proteins.

Applicants co-transfected P19 cells with pGL3 BRE Luciferase and Renilla Luciferase plasmids. After 6 h starvation, cells where pre-incubated with Lefty alone and with increasing concentrations of OMED-001 antibody. After 1 h, cells where induced by 30 minutes with BMP-4. After 24 h, Luciferase activity was measured by Promega's Dual Luciferase Reporter Assay Kit in a VictorNivo plate reader from Perkin Elmer.

FIG. 38 depicts BRE. BMP4 inhibition neutralization by Omed001.

Applicants observed a 30% inhibition of Lefty (5 ug/ml) with the OMed001 antibody (20 ug/ml).

### Example 7 OMED-001 Ab Docking on Lefty1 & Lefty2

The data were generated from the predicted structure of human LEFTY1 and the predicted structure of the variable regions (both heavy and light chains) of OMED-001

The mature chain of Lefty1 and Lefty2 (from aa 77 to 366) is composed of two domains:

1) the TGFbeta propetide domain, from aa 23 to 227, of which the aa 23 to 76 are cleaved out to get the mature chain;

2) The TFGbeta domain, from aa 262 to 353.

The two domains are quite mobile one with respect to the other, possibly with the TGFbeta propetide domain acting as regulator of the TGFbeta domain.

Applicants have considered the docking of the OMED-001, whose Fab region is shown with orange color, onto both the open (shown on the left) and compact (shown on the right) conformation of Lefty1 and Lefty2 (shown in blue), constraining the docking along the Lefty1's peptide PMIVSIKEGGRTRPQVVSLPNMRVQK (SEQ ID NO: 30).

FIG. 39 depicts the best docking conformation of the Ab onto Lefty1 in its open and compact conformations.

In the following Applicants list the map of contacts—i.e. which aa of Lefty 1 and of the Ab are in contact-between the OMED-001 Ab with Lefty1 “open” (first) and Lefty1 “compact” then.

Two residues are defined in contact if any of their heavy atom is within a distance of 5.5 Å.

Applicants refer to the Ab numbering according to the entire FASTA sequence:

(SEQ ID NO: 31)
QVQLQQSGAELVRPGASVKLSCKALGYTFADYEMHWVRQTPVHGLEWTG
SIHPGSGGTAYDQRFKGKATLTADKSSSTAYMELSSLTSEDSAVYYCTF
YDLDYWGQGTTLTVSDILMTQAAPSISVTPGESVSISCRSSESLLHSNG
NTYLYWFLQRPGQSPQLLIYRKSNLASGVPDRFSGSGSGTAFTLRISRV
EAEDVGVYYCMQQLEYPLTFGGGTKLELKRA

where Q in the aa “1”, V is the aa “2”, Q is the aa “3” . . . . R is the aa “226”, A is the (last) aa “227”.

For Lefty 1 and Lefty2 Applicants use the usual numbering from 1 to 366.

The Best docking conformations have been found using HADDOCK first, and than Rosetta for final refinement, overall sampling ˜106600 possible docking complexes.

FIG. 40A depicts a map of contacts between the Ab and the OPEN Lefty1 conformation. The Lefty 1 aa involved in the docking are: 265, 267, 269, 285, 287, 288, 289, 291, 322, 323, 324, 325, 328, 330, 331, 337, 338, 339, 340, 341, 342, 343, 344, 347, 349. Predicted binding affinity (kcal·mol−1): −12.5. Predicted dissociation constant (M) at 25.0° C.: 6.8e-10

FIG. 40B depicts a map of contacts between the Ab and the COMPACT Lefty1 conformation. The Lefty 1 aa involved in the docking are: 276, 277, 278, 279, 280, 281, 328, 329, 330, 331, 332, 333, 334, 335, 336, 339, 340, 341, 342, 343, 344, 345. Predicted binding affinity (kcal·mol−1): −10.6. Predicted dissociation constant (M) at 25.0° C.: 1.8e-08.

FIG. 41 depicts a best docking conformation of the Ab onto Lefty2 in its open and compact conformations.

FIG. 42A depicts a map of contacts between the Ab and the OPEN Lefty2 conformation. The Lefty2 aa involved in the docking are: 275, 276, 277, 280, 281, 320, 321, 322, 323, 324, 325, 328, 329, 330, 331, 332, 333, 334, 335, 337, 339, 340, 341, 342, 343, 344, 345, 346, 347. Predicted binding affinity (kcal·mol−1): −11.9. Predicted dissociation constant (M) at 25.0° C.: 1.9e-09.

FIG. 42B depicts a map of contacts between the Ab and the COMPACT Lefty2 conformation. The Lefty2 aa involved in the docking are: 275, 276, 277, 278, 279, 280, 281, 328, 329, 330, 331, 332, 333, 334, 335, 336, 337, 338, 339, 340, 341, 342, 343, 344, 345, 346. Predicted binding affinity (kcal·mol−1): −12.7. Predicted dissociation constant (M) at 25.0° C.: 4.5e-10.

### REFERENCES

(1970). Liberia: threat to the hippo. Nature 226, 488.
Al-Hajj, M., Wicha, M. S., Benito-Hernandez, A., Morrison, S. J., and Clarke, M. F. (2003). Prospective identification of tumorigenic breast cancer cells. Proc Natl Acad Sci USA 100, 3983-3988.
Alarmo, E. L., and Kallioniemi, A. (2010). Bone morphogenetic proteins in breast cancer: dual role in tumourigenesis? Endocr Relat Cancer 17. R123-139.
Alarmo, E. L., Parssinen, J., Ketolainen, J. M., Savinainen, K., Karhu, R., and Kallioniemi, A. (2009). BMP7 influences proliferation, migration, and invasion of breast cancer cells. Cancer Lett 275, 35-43.
Aykul, S., Ni, W., Mutatu, W., and Martinez-Hackert, E. (2015). Human Cerberus prevents nodal-receptor binding, inhibits nodal signaling, and suppresses nodal-mediated phenotypes. PLOS One 10, e0114954.
Bach, K., Pensa, S., Grzelak, M., and Hadfield, J. (2017). Differentiation dynamics of mammary epithelial cells revealed by single-cell RNA sequencing. 8, 2128.
Balboni, A. L., Hutchinson, J. A., DeCastro, A. J., Cherukuri, P., Liby, K., Sporn, M. B., Schwartz, G. N., Wells, W. A., Sempere, L. F., Yu, P. B., et al. (2013). DeltaNp63alpha-mediated activation of bone morphogenetic protein signaling governs stem cell activity and plasticity in normal and malignant mammary epithelial cells. Cancer Res 73, 1020-1030.
Bandyopadhyay, A., Tsuji, K., Cox, K., Harfe, B. D., Rosen, V., and Tabin, C. J. (2006). Genetic analysis of the roles of BMP2, BMP4, and BMP7 in limb patterning and skeletogenesis. PLOS Genet 2, e216.
Boon, M. R., van der Horst, G., van der Pluijm, G., Tamsma, J. T., Smit, J. W., and Rensen, P. C. (2011). Bone morphogenetic protein 7: a broad-spectrum growth factor with multiple target therapeutic potency. Cytokine Growth Factor Rev 22, 221-229.
Buijs, J. T., Henriquez, N. V., van Overveld, P. G., van der Horst, G., Que, I., Schwaninger, R., Rentsch, C., Ten Dijke, P., Cleton-Jansen, A. M., Driouch, K., et al. (2007). Bone morphogenetic protein 7 in the development and treatment of bone metastases from breast cancer. Cancer Res 67, 8742-8751.
Buijs, J. T., van der Horst, G., van den Hoogen, C., Cheung, H., de Rooij, B., Kroon, J., Petersen, M., van Overveld, P. G., Pelger, R. C., and van der Pluijm, G. (2012). The BMP2/7 heterodimer inhibits the human breast cancer stem cell subpopulation and bone metastases formation. Oncogene 31, 2164-2174.
Cassar, L., Nicholls, C., Pinto, A. R., Chen, R., Wang, L., Li, H., and Liu, J. P. (2017). TGF-beta receptor mediated telomerase inhibition, telomere shortening and breast cancer cell senescence. Protein Cell 8, 39-54.
Cerami, E., Gao, J., Dogrusoz, U., Gross, B. E., Sumer, S. O., Aksoy, B. A., Jacobsen, A., Byrne, C. J., Heuer, M. L., Larsson, E., et al. (2012). The cBio cancer genomics portal: an open platform for exploring multidimensional cancer genomics data. Cancer discovery 2, 401-404.
Chakrabarti, R., Wei, Y., Hwang, J., Hang. X., Andres Blanco, M., Choudhury, A., Tiede, B., Romano, R. A., DeCoste, C., Mercatali, L., et al. (2014). DeltaNp63 promotes stem cell activity in mammary gland development and basal-like breast cancer by enhancing Fzd7 expression and Wnt signalling. Nat Cell Biol 16, 1004-1015, 1001-1013.
Chambers, S. M., Fasano, C. A., Papapetrou, E. P., Tomishima, M., Sadelain, M., and Studer, L. (2009). Highly efficient neural conversion of human ES and iPS cells by dual inhibition of SMAD signaling. Nat Biotechnol 27, 275-280.
Chapellier, M., Bachelard-Cascales, E., Schmidt, X., Clement, F., Treilleux, I., Delay, E., Jammot, A., Menetrier-Caux, C., Pochon, G., Besancon, R., et al. (2015). Disequilibrium of BMP2 levels in the breast stem cell niche launches epithelial transformation by overamplifying BMPR1B cell response. Stem Cell Reports 4, 239-254.
Chen, C., and Shen, M. M. (2004). Two modes by which Lefty proteins inhibit nodal signaling. Curr Biol 14, 618-624.
Cheng, S. K., Olale, F., Brivanlou, A. H., and Schier, A. F. (2004). Lefty blocks a subset of TGFbeta signals by antagonizing EGF-CFC coreceptors. PLOS biology 2, E30.
Curtis, C., Shah, S. P., Chin, S. F., Turashvili, G., Rueda, O. M., Dunning, M. J., Speed, D., Lynch, A. G., Samarajiwa, S., Yuan, Y., et al. (2012). The genomic and transcriptomic architecture of 2,000 breast tumours reveals novel subgroups. Nature 486, 346-352.
Daniel, C. W., Silberstein, G. B., Van Horn, K., Strickland, P., and Robinson, S. (1989). TGF-beta 1-induced inhibition of mouse mammary ductal growth: developmental specificity and characterization. Dev Biol 135, 20-30.
David, C. J., and Massague, J. (2018). Contextual determinants of TGFbeta action in development, immunity and cancer. Nature reviews Molecular cell biology.
de Gramont, A., Faivre, S., and Raymond, E. (2017). Novel TGF-beta inhibitors ready for prime time in onco-immunology. Oncoimmunology 6, e1257453.
Debnath, J., Muthuswamy, S. K., and Brugge, J. S. (2003). Morphogenesis and oncogenesis of MCF-10 A mammary epithelial acini grown in three-dimensional basement membrane cultures. Methods (San Diego, Calif) 30, 256-268.
Deugnier, M. A., Faraldo, M. M., Teuliere, J., Thiery, J. P., Medina, D., and Glukhova, M. A. (2006). Isolation of mouse mammary epithelial progenitor cells with basal characteristics from the Comma-Dbeta cell line. Dev Biol 293, 414-425.
Dichn, M., Cho, R. W., Lobo, N. A., Kalisky, T., Doric, M. J., Kulp, A. N., Qian, D., Lam, J. S., Ailles, L. E., Wong, M., et al. (2009). Association of reactive oxygen species levels and radioresistance in cancer stem cells. Nature 458, 780-783.
Dituri, F., Cossu, C., Mancarella, S., and Giannelli, G. (2019). The Interactivity between TGFbeta and BMP Signaling in Organogenesis, Fibrosis, and Cancer. Cells 8.
Dragu, D. L., Necula, L. G., Bleotu, C., Diaconu, C. C., and Chivu-Economescu, M. (2015). Therapies targeting cancer stem cells: Current trends and future challenges. World J Stem Cells 7, 1185-1201.
Flatz, L., Roychoudhuri, R., Honda, M., Filali-Mouhim, A., Goulet, J. P., Kettaf, N., Lin, M., Roederer, M., Haddad, E. K., Sekaly, R. P., et al. (2011). Single-cell gene-expression profiling reveals qualitatively distinct CD8 T cells elicited by different gene-based vaccines. Proc Natl Acad Sci USA 108, 5724-5729.
Gao, J., Aksoy, B. A., Dogrusoz, U., Dresdner, G., Gross, B., Sumer, S. O., Sun, Y., Jacobsen, A., Sinha, R., Larsson, E., et al. (2013). Integrative analysis of complex cancer genomics and clinical profiles using the cBioPortal. Science signaling 6, pl1.
Gong, W., Sun, B., Sun, H., Zhao, X., Zhang, D., Liu, T., Zhao, N., Gu, Q., Dong, X., and Liu, F. (2017). Nodal signaling activates the Smad2/3 pathway to regulate stem cell-like properties in breast cancer cells. Am J Cancer Res 7, 503-517.
Gordon, K. J., and Blobe, G. C. (2008). Role of transforming growth factor-beta superfamily signaling pathways in human disease. Biochimica et biophysica acta 1782, 197-228.
Guo, G., Huss, M., Tong, G. Q., Wang, C., Li Sun, L., Clarke, N. D., and Robson, P. (2010). Resolution of cell fate decisions revealed by single-cell gene expression analysis from zygote to blastocyst. Developmental cell 18, 675-685.
Heldin, C. H., Miyazono, K., and ten Dijke, P. (1997). TGF-beta signalling from cell membrane to nucleus through SMAD proteins. Nature 390, 465-471.
Hens, J. R., Dann, P., Zhang, J. P., Harris, S., Robinson, G. W., and Wysolmerski, J. (2007). BMP4 and PTHrP interact to stimulate ductal outgrowth during embryonic mammary development and to inhibit hair follicle induction. Development 134, 1221-1230.
Hiremath, M., and Wysolmerski, J. (2013). Parathyroid hormone-related protein specifies the mammary mesenchyme and regulates embryonic mammary development. J Mammary Gland Biol Neoplasia 18, 171-177.
Ikushima, H., and Miyazono, K. (2012). TGF-beta signal transduction spreading to a wider field: a broad variety of mechanisms for context-dependent effects of TGF-beta. Cell Tissue Res 347, 37-49.
Inman, J. L., Robertson, C., Mott, J. D., and Bissell, M. J. (2015). Mammary gland development: cell fate specification, stem cells and the microenvironment. Development 142, 1028-1042.
Jamieson, P. R., Dekkers, J. F., Rios, A. C., Fu, N. Y., Lindeman, G. J., and Visvader, J. E. (2017). Derivation of a robust mouse mammary organoid system for studying tissue dynamics. 144, 1065-1071.
Jung, N., Maguer-Satta, V., and Guyot, B. (2019). Early Steps of Mammary Stem Cell Transformation by Exogenous Signals; Effects of Bisphenol Endocrine Disrupting Chemicals and Bone Morphogenetic Proteins. Cancers (Basel) 11.
Kahata, K., Maturi, V., and Moustakas, A. (2018). TGF-beta Family Signaling in Ductal Differentiation and Branching Morphogenesis. Cold Spring Harbor perspectives in biology 10.
Kenney N, S. G., Johnson M. Rosemberg K, Salomon D S, Dickson R. (1997). Cripto-1 activity in the intact and ovariectomized virgin mouse mammary gland. Pathogenesis, 57-71.
Ketolainen, J. M., Alarmo, E. L., Tuominen, V. J., and Kallioniemi, A. (2010). Parallel inhibition of cell growth and induction of cell migration and invasion in breast cancer cells by bone morphogenetic protein 4. Breast Cancer Res Treat 124, 377-386.
Kim, D. K., Cha, Y., Ahn, H. J., Kim, G., and Park, K. S. (2014). Lefty1 and lefty2 control the balance between self-renewal and pluripotent differentiation of mouse embryonic stem cells. Stem Cells Dev 23, 457-466.
Kirsammer, G., Strizzi, L., Margaryan, N. V., Gilgur, A., Hyser, M., Atkinson, J., Kirschmann, D. A., Seftor, E. A., and Hendrix, M. J. (2014). Nodal signaling promotes a tumorigenic phenotype in human breast cancer. Semin Cancer Biol 29, 40-50.
Klauzinska, M., McCurdy, D., Rangel, M. C., Vaidyanath, A., Castro, N. P., Shen, M. M., Gonzales, M., Bertolette, D., Bianco, C., Callahan, R., et al. (2015). Cripto-1 ablation disrupts alveolar development in the mouse mammary gland through a progesterone receptor-mediated pathway. The American journal of pathology 185, 2907-2922.
Kowanetz, M., Valcourt, U., Bergstrom, R., Heldin, C. H., and Moustakas, A. (2004). Id2 and Id3 define the potency of cell proliferation and differentiation responses to transforming growth factor beta and bone morphogenetic protein. Mol Cell Biol 24, 4241-4254.
Lavery, K., Swain, P., Falb, D., and Alaoui-Ismaili, M. H. (2008). BMP-2/4 and BMP-6/7 differentially utilize cell surface receptors to induce osteoblastic differentiation of human bone marrow-derived mesenchymal stem cells. J Biol Chem 283, 20948-20958.
Lobo, N. A., Shimono, Y., Qian, D., and Clarke, M. F. (2007). The biology of cancer stem cells. Annual review of cell and developmental biology 23, 675-699.
Lobo, N. A., Zabala, M., Qian, D., and Clarke, M. F. (2018). Serially transplantable mammary epithelial cells express the Thy-1 antigen. Breast Cancer Res 20, 121.
Lu. P., and Werb, Z. (2008). Patterning mechanisms of branched organs. Science (New York, NY) 322, 1506-1509.
Marcucci, F., Rumio, C., and Lefoulon, F. (2016). Anti-Cancer Stem-like Cell Compounds in Clinical Development—An Overview and Critical Appraisal. Front Oncol 6, 115.
Massague, J. (2008). TGFbeta in Cancer. Cell 134, 215-230.
Meno, C., Ito, Y., Saijoh, Y., Matsuda, Y., Tashiro, K., Kuhara, S., and Hamada, H. (1997). Two closely-related left-right asymmetrically expressed genes, lefty-1 and lefty-2: their distinct expression domains, chromosomal linkage and direct neuralizing activity in Xenopus embryos. Genes to cells: devoted to molecular & cellular mechanisms 2, 513-524.
Meno, C., Shimono, A., Saijoh, Y., Yashiro, K., Mochida, K., Ohishi, S., Noji, S., Kondoh, H., and Hamada, H. (1998). lefty-1 is required for left-right determination as a regulator of lefty-2 and nodal. Cell 94, 287-297.
Miyazawa, K., Shinozaki, M., Hara, T., Furuya, T., and Miyazono, K. (2002). Two major Smad pathways in TGF-beta superfamily signalling. Genes to cells: devoted to molecular & cellular mechanisms 7, 1191-1204.
Moses, H., and Barcellos-Hoff, M. H. (2011). TGF-beta biology in mammary development and breast cancer. Cold Spring Harb Perspect Biol 3, a003277.
Mou, H., Vinarsky, V., Tata, P. R., Brazauskas, K., Choi, S. H., Crooke, A. K., Zhang, B., Solomon, G. M., Turner, B., Bihler, H., et al. (2016). Dual SMAD Signaling Inhibition Enables Long-Term Expansion of Diverse Epithelial Basal Cells. Cell Stem Cell 19, 217-231.
Naber, H. P., Wiercinska, E., Pardali, E., van Laar, T., Nirmala, E., Sundqvist, A., van Dam, H., van der Horst, G., van der Pluijm, G., Heckmann, B., et al. (2012). BMP-7 inhibits TGF-beta-induced invasion of breast cancer cells through inhibition of integrin beta (3) expression. Cell Oncol (Dordr) 35, 19-28.
Owens, P., Pickup, M. W., Novitskiy, S. V., Chytil, A., Gorska, A. E., Aakre, M. E., West, J., and Moses, H. L. (2012). Disruption of bone morphogenetic protein receptor 2 (BMPR2) in mammary tumors promotes metastases through cell autonomous and paracrine mediators. Proc Natl Acad Sci USA 109, 2814-2819.
Perotti, C., Karayazi, O., Moffat, S., and Shemanko, C. S. (2012). The bone morphogenetic protein receptor-1A pathway is required for lactogenic differentiation of mammary epithelial cells in vitro. In Vitro Cell Dev Biol Anim 48, 377-384.
Petriv, O. I., Kuchenbauer, F., Delaney, A. D., Lecault, V., White, A., Kent, D., Marmolejo, L., Heuser, M., Berg, T., Copley, M., et al. (2010). Comprehensive microRNA expression profiling of the hematopoietic hierarchy. Proc Natl Acad Sci USA 107, 15443-15448.
Pick, E., Moustakas, A., Kurisaki, A., Heldin, C. H., and ten Dijke, P. (1999). TGF-(beta) type I receptor/ALK-5 and Smad proteins mediate epithelial to mesenchymal transdifferentiation in NMuMG breast epithelial cells. J Cell Sci 112 (Pt 24), 4557-4568.
Pietersen, A. M., Evers, B., Prasad, A. A., Tanger, E., Cornelissen-Steijger, P., Jonkers, J., and van Lohuizen, M. (2008). Bmil regulates stem cells and proliferation and differentiation of committed cells in mammary epithelium. Curr Biol 18, 1094-1099.
Plante, P I., Stewart, M. K., and Laird, D. W. (2011). Evaluation of mammary gland development and function in mouse models. Journal of visualized experiments: JoVE.
Prasad, M., Kumar, B., Bhat-Nakshatri, P., Anjanappa, M., Sandusky, G., Miller, K. D., Storniolo, A. M., and Nakshatri, H. (2019). Dual TGFbeta/BMP Pathway Inhibition Enables Expansion and Characterization of Multiple Epithelial Cell Types of the Normal and Cancerous Breast. Mol Cancer Res 17, 1556-1570.
Quail, D. F., Zhang, G., Walsh, L. A., Siegers, G. M., Dieters-Castator, D. Z., Findlay, S. D., Broughton, H., Putman, D. M., Hess, D. A., and Postovit, L. M. (2012). Embryonic morphogen nodal promotes breast cancer growth and progression. PloS one 7, e48237.
Rothenberg, M. E., Nusse, Y., Kalisky, T., Lec, J. J., Dalerba, P., Scheeren, F., Lobo, N., Kulkarni, S., Sim, S., Qian, D., et al. (2012). Identification of a cKit (+) colonic crypt base secretory cell that supports Lgr5 (+) stem cells in mice. Gastroenterology 142, 1195-1205 e1196.
Sato, T., Vries, R. G., Snippert, H. J., van de Wetering, M., Barker, N., Stange, D. E., van Es, J. H., Abo, A., Kujala, P., Peters, P. J., et al. (2009). Single Lgr5 stem cells build crypt-villus structures in vitro without a mesenchymal niche. Nature 459, 262-265.
Schaum, N., Karkanias, J., Neff, N. F., May, A. P., Quake, S. R., Wyss-Coray. T., Darmanis, S., Batson, J., Botvinnik, O., Chen, M. B., et al. (2018). Single-cell transcriptomics of 20 mouse organs creates a Tabula Muris. Nature 562, 367-372.
Scheele, C. L., Hannezo, E., Muraro, M. J., Zomer, A., Langedijk, N. S., van Oudenaarden, A., Simons, B. D., and van Rheenen, J. (2017). Identity and dynamics of mammary stem cells during branching morphogenesis. Nature 542, 313-317.
Shackleton, M., Vaillant, F., Simpson, K. J., Stingl, J., Smyth, G. K., Asselin-Labat, M. L., Wu, L., Lindeman, G. J., and Visvader, J. E. (2006). Generation of a functional mammary gland from a single stem cell. Nature 439, 84-88.
Shi, Y., and Massague, J. (2003). Mechanisms of TGF-beta signaling from cell membrane to the nucleus. Cell 113, 685-700.
Shimono, Y., Zabala, M., Cho, R. W., Lobo, N., Dalerba, P., Qian, D., Dichn, M., Liu, H., Panula, S. P., Chiao, E., et al. (2009). Downregulation of miRNA-200c links breast cancer stem cells with normal stem cells. Cell 138, 592-603.
Sikandar, S. S., Kuo, A. H., Kalisky. T., Cai, S., Zabala, M., Hsich, R. W., Lobo, N. A., and Scheeren, F. A. (2017). Role of epithelial to mesenchymal transition associated genes in mammary gland regeneration and breast tumorigenesis. 8, 1669.
Spike, B. T., Kelber, J. A., Booker, E., Kalathur, M., Rodewald, R., Lipianskaya, J., La, J., He, M., Wright, T., Klemke, R., et al. (2014). CRIPTO/GRP78 signaling maintains fetal and adult mammary stem cells ex vivo. Stem Cell Reports 2, 427-439.
Stingl, J., Eirew, P., Ricketson, I., Shackleton, M., Vaillant, F., Choi, D., Li, H. I., and Eaves, C. J. (2006). Purification and unique properties of mammary epithelial stem cells. Nature 439, 993-997.
Strizzi, L., Postovit, L. M., Margaryan, N. V., Seftor, E. A., Abbott, D. E., Seftor, R. E., Salomon, D. S., and Hendrix, M. J. (2008). Emerging roles of nodal and Cripto-1: from embryogenesis to breast cancer progression. Breast Dis 29, 91-103.
Takahashi, M., Otsuka, F., Miyoshi, T., Otani, H., Goto, J., Yamashita, M., Ogura, T., Makino, H., and Doihara, H. (2008). Bone morphogenetic protein 6 (BMP6) and BMP7 inhibit estrogen-induced proliferation of breast cancer cells by suppressing p38 mitogen-activated protein kinase activation. J Endocrinol 199, 445-455.
Tan, E. J., Olsson, A. K., and Moustakas, A. (2015). Reprogramming during epithelial to mesenchymal transition under the control of TGFbeta. Cell Adh Migr 9, 233-246.
Tiscornia, G., Singer, O., and Verma, I. M. (2006). Production and purification of lentiviral vectors. Nature protocols 1, 241-245.
Ulloa, L., and Tabibzadeh, S. (2001). Lefty inhibits receptor-regulated Smad phosphorylation induced by the activated transforming growth factor-beta receptor. J Biol Chem 276, 21397-21404.
Ventura, A., Meissner, A., Dillon, C. P., McManus, M., Sharp, P. A., Van Parijs, L., Jaenisch, R., and Jacks, T. (2004). Cre-lox-regulated conditional RNA interference from transgenes. Proc Natl Acad Sci USA 101, 10380-10385.
Visser, A. S., Dingemans, K. P., and Prop, F. J. (1981). Morphogenesis in mammary gland cell cultures. Occurrence of tubular structures in combined cultures of fibroblastic and normal epithelial cells. Cell Biol Int Rep 5, 247-251.
Visvader, J. E., and Stingl, J. (2014). Mammary stem cells and the differentiation hierarchy: current status and perspectives. Genes & development 28, 1143-1158.
Wang. N., Lin, K. K., Lu, Z., Lam, K. S., Newton, R., Xu, X., Yu, Z., Gill, G. N., and Andersen, B. (2007). The LIM-only factor LMO4 regulates expression of the BMP7 gene through an HDAC2-dependent mechanism, and controls cell proliferation and apoptosis of mammary epithelial cells. Oncogene 26, 6431-6441.
Wechselberger, C., Ebert, A. D., Bianco, C., Khan, N. I., Sun, Y., Wallace-Jones, B., Montesano, R., and Salomon, D. S. (2001). Cripto-1 enhances migration and branching morphogenesis of mouse mammary epithelial cells. Experimental cell research 266, 95-105.
Welm, B. E., Dijkgraaf, G. J., Bledau, A. S., Welm, A. L., and Werb, Z. (2008). Lentiviral transduction of mammary stem cells for analysis of gene function during development and cancer. Cell Stem Cell 2, 90-102.
Yan, W., and Chen, X. (2007). Targeted repression of bone morphogenetic protein 7. a novel target of the p53 family, triggers proliferative defect in p53-deficient breast cancer cells. Cancer Res 67, 9117-9124.
Ych, L. C. (2010). In vitro and in vivo studies on the effects of bone morphogenetic protein-7 on human kidney and lung tumor cells. International journal of biomedical science: IJBS 6, 176-181.
Yeo, C., and Whitman, M. (2001). Nodal signals to Smads through Cripto-dependent and Cripto-independent mechanisms. Mol Cell 7, 949-957.
Ying. X., Sun, Y., and He, P. (2017). MicroRNA-137 inhibits BMP7 to enhance the epithelial-mesenchymal transition of breast cancer cells. Oncotarget 8, 18348-18358.
Yu, P. B., Beppu, H., Kawai, N., Li, E., and Bloch, K. D. (2005). Bone morphogenetic protein (BMP) type II receptor deletion reveals BMP ligand-specific gain of signaling in pulmonary artery smooth muscle cells. J Biol Chem 280, 24443-24450.
Zeisberg, M., Hanai, J., Sugimoto, H., Mammoto, T., Charytan, D., Strutz, F., and Kalluri, R. (2003). BMP-7 counteracts TGF-beta1-induced epithelial-to-mesenchymal transition and reverses chronic renal injury. Nat Med 9, 964-968.
Zeng. Y. A., and Nusse, R. (2010). Wnt proteins are self-renewal factors for mammary stem cells and promote their long-term expansion in culture. Cell Stem Cell 6, 568-577.
Zinski, J., Tajer, B., and Mullins, M. C. (2018). TGF-beta Family Signaling in Early Vertebrate Development. Cold Spring Harbor perspectives in biology 10.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1-31. (canceled)

32. An antibody or antigen binding fragment thereof, that binds to and neutralises LEFTY1 protein (SEQ ID NO: 32), comprising a heavy chain variable region and a light chain variable region,
(a) wherein the heavy chain variable region comprises heavy chain CDRs comprising the amino acid sequences HCDR1 DYEMH (SEQ ID NO: 6) or HCDR1 DYEIH (SEQ ID NO: 7), HCDR2 SIHPGSGGTAYAQKFQG (SEQ ID NO: 8) and HCDR3 YDLDY (SEQ ID NO: 9), and wherein the light chain variable region comprises light chain CDRs comprising the amino acid sequences of LCDR1 RSSESLLHSNGNTYLY (SEQ ID NO: 10) or LCDR1 RSSESLLHSIGKTYLY (SEQ ID NO: 11), LCDR2 RKSNLAS (SEQ ID NO: 12) and LCDR3 MQQLEYPLT (SEQ ID NO: 13), or,
(b) wherein the heavy chain variable region comprises heavy chain CDRs comprising the amino acid sequences HCDR1 GFSFSSSYW (SEQ ID NO: 1), HCDR2 IYAGSTGTT (SEQ ID NO: 2) and HCDR3 ARGDYNSGWGVNL (SEQ ID NO: 3), and wherein the light chain variable region comprises light chain CDRs comprising the amino acid sequences of LCDR1 ESISSN (SEQ ID NO: 4), LCDR2 SAS and LCDR3 QCTDYVNSGA (SEQ ID NO: 5).

33. The antibody or antigen binding fragment thereof of claim 32, wherein the heavy chain comprises a sequence at least 95% identical to the heavy chain of FIG. 35 and the light chain comprises a sequence at least 95% identical to the light chain of FIG. 35.

34. The antibody or antigen binding fragment thereof of claim 32, wherein the heavy chain variable region comprises a sequence at least 95% identical to the heavy chain variable region of FIG. 25A and the light chain variable region comprises a sequence at least 95% identical to the light chain variable region of FIG. 25A.

35. The antibody or antigen binding fragment thereof of claim 32, wherein the heavy chain comprises a sequence at least 95% identical to the heavy chain of FIG. 27 and the light chain comprises a sequence at least 95% identical to the light chain of FIG. 27.

36. The antibody or antigen binding fragment thereof according to claim 32, wherein the heavy chain variable region and the light chain variable region are separated by a linker in a single polypeptide chain.

37. The antibody or antigen binding fragment thereof of claim 36, wherein the antibody or antigen binding fragment thereof is a (i) a single chain Fv fragment (scFv); (ii) a dimeric scFv (di-scFv); (iii) a trimeric scFv (tri-scFv); (iv) any one of (i), (ii) or (iii) linked to a constant region of an antibody, Fc or a heavy chain constant domain CH2 and/or CH3.

38. The antibody or antigen binding fragment thereof according to claim 32, wherein the heavy chain variable region and the light chain variable region are in separate polypeptide chains, wherein the antibody or antigen binding fragment thereof is a (i) a diabody; (ii) a triabody; (iii) a tetrabody; (iv) a Fab; (v) a F(ab′)2; (vi) a Fv; (vii) one of (i) to (vi) linked to a constant region of an antibody, Fc or a heavy chain constant domain CH2 and/or CH3; or, (viii) an intact antibody.

39. The antibody or antigen binding fragment thereof of claim 38, wherein the neutralizing antibody comprises a wild type IgG1 Fc sequence or an amino acid variant thereof.

40. An antibody or antigen binding fragment thereof, that binds to LEFTY1 protein, comprising heavy chain variable region according to SEQ ID NO: 81 (QVQLVQSGAEVDYEMHWVRQAPGQGLEWTGKKPGASVKVSCKASGYTFADYEMH DYEMHSIHPGSGGTAYAQKFQGRVTSTRDTSISTAYMELSSLRSDDTVVYYCTF YDLDYWGQGTTVTVSS) or a sequence having at least 95% identity to SEQ ID NO: 81 and a light chain variable region according to SEQ ID NO: 82 (DIVLTQSPSSLSVSPGERVTISC RSSESLLHSNGNTYLYWYQQKPGQAPKLLIYRKSNLASGVPDRESGSGSGTDFTLTISGL QAEDEAVYYCMQQLEYPLTFGGGTKLEIKRTV) or a sequence having at least 95% identity to SEQ ID NO: 82.

41. A nucleic acid encoding an antibody or antigen binding fragment thereof according to claim 32.

42. An expression construct comprising a nucleic acid of claim 41.

43. An isolated or recombinant cell expressing an antibody or antigen binding fragment thereof of claim 32.

44. An isolated or recombinant cell comprising a nucleic acid of claim 41 or an expression vector construct of claim 42.

45. A composition comprising an antibody or antigen binding fragment thereof of claim 32 and a pharmaceutically acceptable carrier.

46. A method of treating cancer, comprising administering an effective amount of the composition of claim 45 to a subject in need thereof.

47. The method of claim 46, wherein administering the composition increases expression or activating SMAD2 and/or SMAD5 in the subject.

48. The method of claim 46, wherein the antibody or antigen fragment thereof neutralizes LEFTY protein in the subject.

49. The method of claim 48, wherein the LEFTY protein is LEFTY1, LEFTY2, LEFTY-A or LEFTY-B.

50. The method of claim 46, wherein the cancer is selected from breast invasive carcinoma, liver carcinoma, cholangiocarcinoma, uterine carcinoma, ovarian carcinoma, melanoma, thymoma, lung adenocarcinoma, pheochromocytoma/paraganglioma, esophageal carcinoma, pancreatic carcinoma, glioblastoma multiforme, colorectal carcinoma, renal cell carcinoma or adrenal carcinoma.

51. An antibody or antigen binding fragment thereof comprising a peptide sequence that binds to and neutralized LEFTY1 protein (SEQ ID NO: 32) wherein the sequence binds to the C-terminal region of the LEFTY1 protein comprising residues 320 to 366 of the LEFTY1 protein.
